(19) United States
(12) Patent Application Publication
(10) Pub. No.: US 2021/0085607 A1
(43) Pub. Date: Mar. 25, 2021

(54) CONTINUOUS MICROPARTICLE MANUFACTURE

(71) Applicant: Graybug Vision, Inc., Redwood City, CA (US)

(72) Inventors: Daniel Saragnese, Baltimore, MD (US); Ming Yang, Lutherville-Timonium, MD (US); Yun Yu, Baltimore, MD (US); Toni-Rose Guiriba, Redwood City, CA (US); David McKenzie, Redwood City, CA (US)

(73) Assignee: Graybug Vision, Inc., Redwood City, CA (US)

(21) Appl. No.: 17/077,856

(22) Filed: Oct. 22, 2020

Related U.S. Application Data
(63) Continuation of application No. PCT/US2019/028803, filed on Apr. 23, 2019.
(60) Provisional application No. 62/661,561, filed on Apr. 23, 2018.
(60) Provisional application No. 62/661,563, filed on Apr. 23, 2018.
(60) Provisional application No. 62/661,566, filed on Apr. 23, 2018.

(51) Int. Cl. A61K 9/16; A61K 31/404

(52) U.S. Cl. CPC A61K 9/1682; A61K 9/1629; A61K 31/404

(57) ABSTRACT

The present invention is in the field of manufacturing drug-loaded microparticles, and specifically provides processes for producing approximately homogenously sized drug loaded microparticles with high drug loading and reproducible drug release profiles, and which may be provided in a significantly reduced time period.

### CROSS-REFERENCE TO RELATED APPLICATIONS

This application is a continuation of International Patent Application No. PCT/US2019/028803, filed in the U.S. Receiving Office on Apr. 23, 2019, which claims the benefit of provisional U.S. Application No. 62/661,561, filed Apr. 23, 2018; U.S. Application No. 62/661,563, filed Apr. 23, 2018; and U.S. Application No. 62/661,566, filed Apr. 23, 2018. The entirety of each of these applications is incorporated herein by reference.

### FIELD OF THE INVENTION

The present invention is in the field of manufacturing drug-loaded microparticles, and specifically provides processes for producing approximately homogenously sized drug loaded microparticles with high drug loading and reproducible drug release profiles, and which may be provided in a significantly reduced time period.

### BACKGROUND OF THE INVENTION

Biodegradable polymers provide an established route for the delivery of drugs in a controlled and targeted manner. Substantial release of encapsulated drug molecules from biodegradable polymers is achieved by degradation and erosion of the polymer matrix. One strategy used to produce sustained-release dosage forms involves encapsulation of drug compounds within biodegradable polymeric microparticles or microspheres. These drug-encapsulating microparticles have the potential to provide a more controlled route to adjust release rates than other types of formulations.

Various processes are known to encapsulate a drug within a polymeric microparticle. One process is based upon the initial formation of an emulsion, wherein the drug to be encapsulated is dissolved in a solvent along with the polymer, forming a dispersed phase. The dispersed phase is then mixed with a second solvent called the continuous phase to form an emulsion. Depending upon the conditions used, microparticles may form at this stage or may benefit from additional induction steps. One example of an additional induction step involves the addition of a third extraction solvent to remove solvent from the microdroplets in the emulsion, leading to their subsequent hardening to microparticles. Upon formation, the microparticles generally remain suspended in solvent, which must be removed using additional processing steps to achieve a final product suitable for delivery.

Early approaches to remove solvent involved evaporation, for example by application of vacuum, heat, or compressed air. This approach, however, is time consuming and impractical when performed on a large scale. Extraction has been proposed as an alternative solvent removal process for large scale continuous production of microparticles.

For example, U.S. Pat. No. 8,703,843, assigned to Evonik Corporation, describes a process for the formation of microparticles. First, an emulsion between a first phase containing the active agent and a polymer and a continuous process medium is formed. Subsequently, an extraction phase is added that extracts the first solvent, leading to the formation of microparticles. U.S. Pat. No. 6,495,166, assigned to Alkermes Controlled Therapeutics Inc., describes the formation of an emulsion by the combination of a first phase containing the active agent, polymer, and solvent with a second phase in a first static mixer to form an emulsion. Subsequent combination of the emulsion with a first extraction liquid occurs in a second static mixer. U.S. Pat. No. 6,440,493, assigned to Southern Biosystems, Inc., describes a process initially comprising the formation of an emulsion upon mixing of a dispersed phase and a continuous phase. Microparticles are formed upon addition of an extraction phase to the emulsion, and a subsequent evaporation stage removes substantially all of the solvent remaining in the microparticles. U.S. Pat. No. 5,945,126, assigned to Oakwood Laboratories, L.L.C., describes the formation of an emulsion of a dispersed phase and continuous phase by slow addition of both phases simultaneously to a reactor undergoing intense mixing to provide high shear, coinciding with continuous transportation of the formed emulsion to a solvent removal vessel. U.S. Patent Publication No. 2010/0143479, assigned to Oakwood Laboratories LLC, describes a process for the formation of a microparticle dispersion upon mixing of a dispersed phase and a continuous phase to form a microparticle dispersion, followed by the addition of a dilution composition to the microparticle dispersion.

Despite these advances, these processes often result in microparticles with (i) low drug loading, (ii) particle instability, and/or (iii) inadequate control of drug release profiles. It is an objective of the present invention to provide processes and systems that reduce residence time of drug-loaded microparticles and allow for the production of more stable, homogeneously-sized microparticles with high drug loadings and/or reproducible release profiles, and the microparticles prepared thereby.

### SUMMARY OF THE INVENTION

The present invention provides processes and systems for the production of microparticles resulting in significantly reduced residence time of the formed microparticle in the presence of solvent. Accordingly, the present invention provides more consistent batches of microparticles with high levels of drug loading and controllable drug release profiles.

In one aspect of the present invention, the process includes a bank of centrifuges or continuous liquid centrifuge in the processing of microparticles after formation that allows for rapid removal of solvent from the liquid dispersion in a timely manner, while the number of processing steps and time necessary to produce a drug-loaded microparticle suitable for therapeutic administration is reduced. By using centrifugation techniques in a continuous process, higher amounts of supernatant-containing solvent can be removed during a single pass in a shorter amount of time compared to other microparticle purification techniques.

In another aspect of the present invention, a thick wall hollow fiber tangential flow filter (TWHFTFF) is used in combination with a plug flow reactor. By combining a plug flow reactor that provides controlled exposure time to a solvent extraction phase for solvent removal directly in tandem with a high evacuation, macro-filtration device such as a thick wall hollow fiber tangential flow filter (TWHFTFF), rapid removal of solvent from the liquid dispersion is accomplished in a timely manner, while the number of processing steps and time necessary to produce a drug-loaded microparticle suitable for therapeutic administration is reduced.

In yet a further aspect of the present invention, the process includes a microfluidic droplet generator in combination with centrifuge, plug flow reactor and/or macro-filtration device such as a thick wall hollow fiber tangential flow filter (TWHFTFF). The microfluidic droplet generator generates significantly less solvent than commonly used processes for microparticle formation and is advantageous compared to other commonly used methods due to its efficiency, its rapid removal and minimal consumption of solvent, and its ability to consistently produce highly monodisperse particles.

Microparticle production techniques often result in microparticle batches of varying size, drug loading, and stability. Administering microparticles with inconsistent properties results in inconsistent drug release, biodegradability, and overall efficacy. Therefore, microparticle processes that do not provide predictable and consistently sized microparticles require further processing, which often involves additional solvent exposure time and therefore, increased drug leaching. Decreased drug loading as a result of drug leaching in the production process can negatively affect extended drug release and the potential therapeutic efficiency of the microparticles. Therefore, a process that decreases solvent exposure time while simultaneously removing microparticles of an undesirable size are advantageous to these prior art processes. As discussed in Example 4 and shown in FIG. 1M, FIG. 1N, and FIG. 1O, continuous centrifugation effectively removes small, non-desired microparticles during processing. As exhibited herein as one non-limiting example, prior to centrifugation, particles less than 10 μm comprised 6.8% of the total particle size distribution. The percent of particles less than 10 μm was decreased by 21% after only one round of centrifugation. The fraction of small particles was further reduced with subsequent centrifugation and after three rounds particles less than 10 μm comprised only 2.7% of the total particles. This corresponded to a 60% reduction in the percent of particles less than 10 μm compared with no centrifugation (FIG. 1M).

Continuous or Parallel Centrifugation

The present invention provides processes and systems for the production of microparticles by using specific centrifugation techniques that allow high throughput processing of the microparticles in a continuous manner. In one aspect, the processes and systems provided by the present invention use a parallel bank of centrifuges to remove solvent from the microparticles produced in a continuous process. Alternatively, the processes provide for the use of a continuous liquid centrifuge, such as a solid bowl or conical plate centrifuge, to allow continuous and simultaneous removal of both waste solvent liquid and microparticles of an undesired size. Both of these centrifugation systems can also significantly reduce the residence time of the formed microparticles in residual solvent, reducing the incidence of leaching in drug-loaded microparticles.

In one aspect of the present invention, provided herein is a process of producing drug-loaded microparticles in a continuous process which includes: a) continuously forming an emulsion comprising a dispersed phase and a continuous phase in a mixer, wherein the dispersed phase comprises a drug, a polymer, and at least one solvent; b) directly feeding the emulsion into a quench vessel, whereupon entering the quench vessel the emulsion is mixed with an extraction phase to form a liquid dispersion, wherein a portion of the solvent is extracted into the extraction phase and microparticles are formed; c) continuously feeding the liquid dispersion from the quench vessel into a parallel bank of centrifuges via an outlet from the quench vessel, wherein a portion of the liquid dispersion containing solvent and microparticles below a specified size threshold are removed with a waste solvent liquid and remaining microparticles above the specified size threshold are isolated as a concentrated slurry; and d) transferring the concentrated slurry from the centrifuge to a receiving vessel for further processing, if desired. In some embodiments, the liquid dispersion from the outlet of the quench vessel is diverted to a first centrifuge in a parallel bank of two or more centrifuges. After a set centrifugation time, the liquid dispersion from the outlet of the quench vessel is diverted into a one or more additional centrifuges instead of the first centrifuge. In some embodiments, the concentrated slurry is optionally rinsed with a wash phase while residing in the centrifuge. In some embodiments, the concentrated slurry present within the first centrifuge is optionally rinsed with a wash phase while the liquid dispersion is being diverted to one or more additional centrifuges within the parallel bank. In another embodiment, the liquid dispersion from the quench vessel is run through two or more centrifuges operating simultaneously in a parallel bank of centrifuges. In some embodiments, the two or more centrifuges operate in alternate. In some embodiments, the two or more centrifuges are arranged serially. In some embodiments, the concentrated slurry in the receiving vessel is optionally diluted with a wash phase and returned to the parallel bank of centrifuges for additional processing. In some embodiments, the quench vessel is a plug flow reactor.

In one aspect of the present invention, provided herein is a process of producing drug-loaded microparticles in a continuous process which includes: a) continuously forming an emulsion comprising a dispersed phase and a continuous phase in a mixer, wherein the dispersed phase comprises a drug, a polymer, and at least one solvent; b) directly feeding the emulsion into a quench vessel, whereupon entering the quench vessel the emulsion is mixed with an extraction phase to form a liquid dispersion, whereupon a portion of the solvent is extracted into the extraction phase and microparticles are formed; c) continuously feeding the liquid dispersion from the quench vessel into a continuous liquid centrifuge via an outlet from the quench vessel, wherein a portion of the liquid dispersion containing solvent and microparticles below a specified size threshold are removed with a waste solvent liquid and remaining microparticles above the specified size threshold are isolated as a concentrated slurry; and d) continuously transferring the concentrated slurry from the centrifuge to a receiving vessel for further processing, if desired. In some embodiments, the continuous liquid centrifuge is a solid bowl centrifuge. In another embodiment, the continuous liquid centrifuge is a conical plate centrifuge. In some embodiments, the concentrated slurry is optionally rinsed with a wash phase while residing in the centrifuge. In some embodiments, the concentrated slurry in the receiving vessel is optionally diluted with a wash phase and returned to the continuous liquid centrifuge for additional processing. In some embodiments, the quench vessel is a reactor filter. In some embodiments, the quench vessel is a plug flow reactor.

Upon reaching the receiving vessel as provided for in the above embodiments, the microparticles can be further processed, for example by continuous recirculation from the receiving vessel through one or more centrifuges to further remove solvent and microparticles of undesirable size. In some embodiments, the receiving vessel is pre-filled with a wash phase. In some embodiments, additional extraction phase is simultaneously added to the receiving vessel upon transfer of the concentrated slurry. In some embodiments, the receiving vessel is pre-filled with a wash phase, and, as the concentrated slurry enters the receiving vessel, additional wash phase is also continuously added. In certain embodiments, sufficient wash phase is added to the concentrated slurry in the centrifuge so that additional wash phase is not required during the remainder of the process, for example, upon entry into the receiving vessel. In some embodiments, one or more additional washes of the microparticles or one or more additional formulation steps may be performed on the concentrated slurry in the receiving vessel.

In one aspect of the present invention, a surface treatment phase may be optionally added to the liquid dispersion of microparticles while present within the quench vessel. The surface treatment is typically added to facilitate aggregation of the formed microparticles when used in their intended application. In another aspect, a surface treatment phase may be optionally added to the concentrated slurry of microparticles when present within the centrifuge. In yet another aspect of the present invention, a surface treatment phase may be optionally added to the concentrated slurry of microparticles when present within the receiving vessel.

Various types of centrifuges may be used in any embodiments of the present invention. In some embodiments, the centrifuge is a filtration centrifuge. In some embodiments, the filtration centrifuge is selected from a conveyer discharge centrifuge, a pusher centrifuge, a peeler centrifuge, an inverting filter centrifuge, a sliding discharge centrifuge, and a pendulum centrifuge fitted with a perforated drum. In another embodiment, the centrifuge is a sedimentation centrifuge. In some embodiments, the sedimentation centrifuge is selected from a pendulum centrifuge fitted with a solid drum, a solid bowl centrifuge, a conical plate centrifuge, a tubular centrifuge, and a decanter centrifuge. In some embodiments, the centrifuge is an overflow centrifuge that allows continual removal of supernatant from the added liquid dispersion.

By using either a parallel bank of centrifuges or a continuous liquid centrifuge, residence time of the microparticles with extraction phase can be more tightly controlled. Thus, desirable microparticle drug elution characteristics can be derived and maintained by the high rate supernatant removal provided by the centrifuge and the subsequent further dilution of solvent through the exposure of the microparticles to further extraction phase in the receiving vessel. Because the process provides for a higher throughput due to the higher rate of supernatant removal, and thus a quicker processing time, the formed microparticles are less susceptible to further drug elution due to residual solvent presence and/or, in the case of highly hydrophilic drugs, extended residence in the extraction solvent.

Thick Wall Hollow Fiber Tangential Flow Filter (TWHFTFF)

In one aspect of the present invention, provided herein is a process of producing drug-loaded microparticles in a continuous process which includes: a) continuously forming an emulsion comprising a dispersed phase and a continuous phase in a mixer, wherein the dispersed phase comprises a drug, a polymer, and at least one solvent; b) directly feeding the emulsion into a plug flow reactor, wherein upon entering the plug flow reactor, the emulsion is mixed with a solvent extraction phase to form a liquid dispersion, wherein during residence in the plug flow reactor, a portion of the solvent is extracted into the extraction phase and the microparticles are hardened; c) directly feeding the liquid dispersion to a TWHFTFF, wherein the TWHFTFF is directly in-tandem with the plug flow reactor, and wherein a portion of the liquid dispersion containing solvent and microparticles below a specified-size threshold are removed as a permeate; and d) transferring the retentate to a holding tank. In some embodiments, additional extraction phase is introduced into the plug flow reactor at one or more locations as the liquid dispersion traverses through the reactor so that a serial extraction of solvent occurs.

In an alternative aspect of the present invention, provided herein is a process of producing drug-loaded microparticles in a continuous process which includes: a) continuously forming an emulsion comprising a dispersed phase and a continuous phase in a mixer, wherein the dispersed phase comprises a drug, a polymer, and at least one solvent; b) directly feeding the emulsion into a quench vessel, whereupon entering the quench vessel the emulsion is mixed with an extraction phase to form a liquid dispersion, whereupon a portion of the solvent is extracted into the extraction phase and microparticles are formed; c) continuously feeding the liquid dispersion from the quench vessel into a continuous liquid centrifuge via an outlet from the quench vessel, wherein a portion of the liquid dispersion containing solvent and microparticles below a specified size threshold are removed with a waste solvent liquid and remaining microparticles above the specified size threshold are isolated as a concentrated slurry; and d) continuously recirculating the concentrated slurry from the continuous liquid centrifuge to the quench vessel, whereupon entering the quench vessel, the concentrated slurry is rinsed with water or mixed with surface treatment phase; e) continuously transferring the microparticles from the liquid centrifuge to a receiving vessel for further processing, if desired. In some embodiments, the continuous liquid centrifuge is a solid bowl centrifuge. In another embodiment, the continuous liquid centrifuge is a conical plate centrifuge. In some embodiments, the concentrated slurry is optionally rinsed with a wash phase while residing in the centrifuge. In some embodiments, the receiving vessel is connected to a thick wall hollow fiber tangential flow filter (TWHFTFF).

In an alternative aspect, the process of producing drug-loaded microparticles in a continuous process includes a) continuously forming an emulsion comprising a dispersed phase and a continuous phase in a mixer, wherein the dispersed phase comprises a drug, a polymer, and at least one solvent; b) directly feeding the emulsion into a quench vessel, whereupon entering the quench vessel the emulsion is mixed with an extraction phase to form a liquid dispersion, whereupon a portion of the solvent is extracted into the extraction phase and microparticles are formed; c) continuously feeding the liquid dispersion from the quench vessel into a continuous liquid centrifuge via an outlet from the quench vessel, wherein a portion of the liquid dispersion containing solvent and microparticles below a specified size threshold are removed with a waste solvent liquid and remaining microparticles above the specified size threshold are isolated as a concentrated slurry; and, d) continuously recirculating the concentrated slurry from the continuous liquid centrifuge to the quench vessel, whereupon entering the quench vessel, the concentrated slurry is rinsed with water or mixed with surface treatment phase; e) directly feeding the liquid dispersion to a reactor vessel connected to a TWHFTFF, wherein a portion of the liquid dispersion containing solvent and microparticles below a specified-size threshold are removed as a permeate; and e) transferring the retentate to a holding tank.

Microfluidic Droplet Generator

In one aspect of the present invention, provided herein is a process of producing drug-loaded microparticles in a continuous process which includes a) continuously combining a dispersed phase and a continuous phase in a microfluidic droplet generator to produce droplets, wherein the dispersed phase comprises a drug, a polymer, and at least one solvent; b) directly feeding the droplets into a plug flow reactor, wherein upon entering the plug flow reactor, the droplets are mixed with a solvent extraction phase, wherein during residence in the plug flow reactor, a portion of the solvent is extracted into the extraction phase and the droplets are hardened to produce microparticles; c) exposing the microparticles to surface-treatment solution in the plug flow reactor to produce surface-treated microparticles, d) directly feeding the microparticle suspension into a dilution vessel wherein the microparticles are washed and diluted to a target filling concentration; and e) transferring the diluted microparticle suspension into an apparatus designed for a filling operation.

In another aspect of the present invention, a parallel bank of centrifuges or a continuous liquid centrifuge is used in conjugation with a microfluidic droplet generator. In this embodiment, the process of producing drug-loaded microparticles in a continuous process includes a) continuously combining a dispersed phase and a continuous phase in a microfluidic droplet generator to produce droplets, wherein the dispersed phase comprises a drug, a polymer, and at least one solvent; b) directly feeding the droplets into a plug flow reactor, wherein upon entering the plug flow reactor, the droplets are mixed with a solvent extraction phase, wherein during residence in the plug flow reactor, a portion of the solvent is extracted into the extraction phase and the droplets are hardened to produce microparticles; c) exposing the microparticles to surface-treatment solution in the plug flow reactor to produce surface-treated microparticles, d) directly feeding the liquid dispersion to a reactor vessel connected to a continuous liquid centrifuge or a parallel bank of centrifuges via an outlet from the reactor vessel, wherein a portion of the liquid dispersion containing solvent and microparticles below a specified size threshold are removed with a waste solvent liquid and remaining microparticles above the specified size threshold are isolated as a concentrated slurry; and e) transferring the concentrated slurry into an apparatus designed for a washing and filling operation.

In some embodiments, the microfluidic droplet generator further comprises a turbulence based micro-mixing channel.

### BRIEF DESCRIPTION OF THE FIGURES

FIG. 1A shows a schematic of a process for producing a microparticle by utilizing centrifugation techniques as described herein.

FIG. 1B shows a schematic of an exemplary continuous liquid centrifuge to be used according the embodiments of the invention.

FIG. 1C shows a schematic of an exemplary centrifuge to be used according the embodiments of the invention.

FIG. 1D shows a schematic of a system for producing a microparticle according to embodiments of the invention that utilize centrifugation techniques.

FIG. 1E shows a schematic of an exemplary plug flow reactor that can be used as a quench vessel according to embodiments of the invention.

FIG. 1F shows a schematic of a series of plug flow reactors with static mixers in-between that is used as a quench vessel according to embodiments of the invention.

FIG. 1G shows a schematic of an exemplary bank of centrifuges that can be used in the system according to the embodiments of the invention.

FIG. 1H shows a schematic of a holding tank used in producing a microparticle according to embodiments of the invention.

FIG. 1I shows a schematic of a process for producing a microparticle by utilizing centrifugation techniques as described herein in conjunction with a thick wall hollow fiber tangential flow filter.

FIG. 1J shows an exemplary schematic of a process for producing a microparticle by utilizing centrifugation techniques as described herein in conjunction with a thick wall hollow fiber tangential flow filter.

FIG. 1K shows an exemplary schematic of a process for producing a microparticle by utilizing centrifugation techniques as described herein in conjunction with a thick wall hollow fiber tangential flow filter.

FIG. 1L shows an exemplary schematic of a process for producing a microparticle by utilizing centrifugation techniques as described herein.

FIG. 1M is a diagram illustrating the impact of continuous centrifugation as described in Example 4. After each centrifugation, the volume of microparticles with diameters less than 10 μm decreases. Before any centrifugation, particles less than 10 μm comprised 8.6% of the total size distribution, but after four rounds of centrifugation, a 68% reduction in the percent of particles smaller than 10 μm was observed. The x-axis is particle diameter measured in μm and the y-axis is the differential volume of microparticles of different sizes measured in percent.

FIG. 1N is a diagram illustrating the impact of continuous centrifugation on the supernatant of the microparticle suspension as described in Example 4. After each round of centrifugation, the percentage of particles smaller than 10 μm was observed. The x-axis is particle diameter measured in μm and the y-axis is the differential volume of microparticles of different sizes measured in percent.

FIG. 1O is a diagram illustrating the impact of continuous centrifugation as described in Example 4. After continuous centrifugation, the volume of microparticles with diameters less than 10 μm decreases. The amount of small particles less than 10 μm in the final product was 69% lower than that prior to centrifugation. The x-axis is particle diameter measured in μm and the y-axis is the differential volume of microparticles of different sizes measured in percent.

FIG. 2A shows a schematic of a process for producing a microparticle by utilizing a plug flow reactor in combination with a thick wall hollow fiber tangential flow filter.

FIG. 2B shows a schematic of a system for producing a microparticle according to embodiments of the invention that utilize a plug flow reactor in combination with a thick wall hollow fiber tangential flow filter.

FIG. 2C shows a schematic of a plug flow reactor used in producing a microparticle according to embodiments of the invention.

FIG. 2D shows a schematic of a plug flow reactor with multiple addition points for extraction solvent that is used in producing a microparticle according to the embodiments of the invention.

FIG. 2E shows a schematic of a series of plug flow reactors with static mixer in-between that is used in producing a microparticle according to the embodiments of the invention.

FIG. 2F shows a schematic of a holding tank used in producing a microparticle according to embodiments of the invention.

FIG. 3A shows a schematic of a process for producing a microparticle according to embodiments of the invention wherein the microfluidic droplet generator forms droplets in a liquid suspension.

FIG. 3B shows a schematic of a system for producing a microparticle according to embodiments of the invention wherein the microfluidic droplet generator has a T-junction.

FIG. 3C shows a schematic of a microfluidic droplet generator with a T-junction used in producing a microparticle according to embodiments of the invention.

FIG. 3D shows a schematic of a 4-pronged microfluidic droplet generator used in producing a microparticle according to embodiments of the invention.

FIG. 3E shows a schematic for producing a microparticle where two microfluidic droplet generators are used in producing a microparticle according to embodiments of the invention.

FIG. 3F shows a schematic of plug flow reactor with two inlets and two holding tanks used in producing a microparticle according to embodiments of the invention.

FIG. 3G shows a schematic of plug flow reactor with three inlets and three holding tanks used in producing a microparticle according to embodiments of the invention.

FIG. 3H shows a schematic of a series of plug flow reactors in direct fluid communication via a series of static mixers.

FIG. 3I shows a schematic of dilution vessel attached to two vessels for producing a microparticle according to embodiments of the invention.

FIG. 3J shows a schematic of a system for producing a microparticle according to embodiments of the invention utilizing a microfluidic droplet generator in conjunction with centrifugation

### DETAILED DESCRIPTION OF THE INVENTION

Provided herein are processes and systems for producing microparticles in a continuous, high-throughput manner. These processes provide consistent batches of microparticles with high levels of drug loading and consistent, controllable drug release profiles. By using the processes and systems described herein, microparticles with high drug loading capacity and/or desirable drug release profiles can be produced.

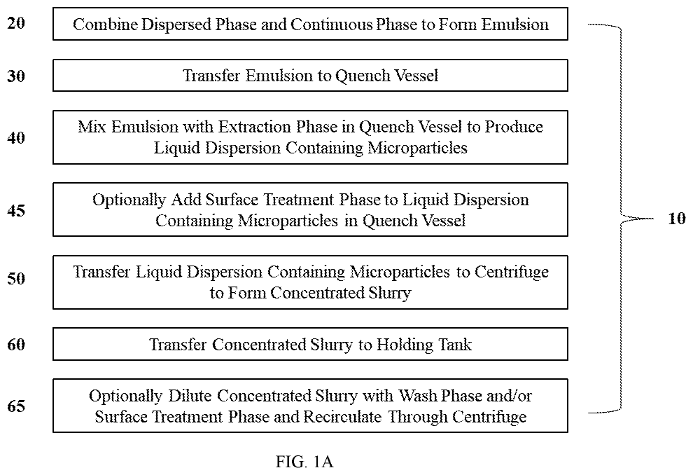

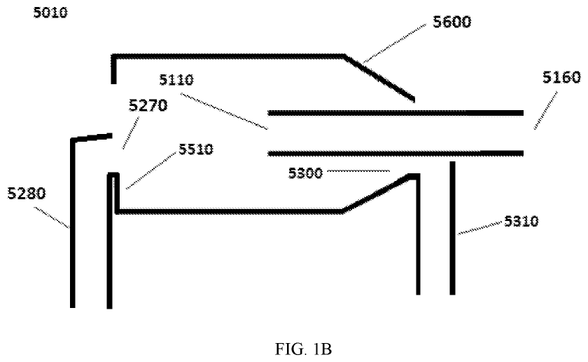

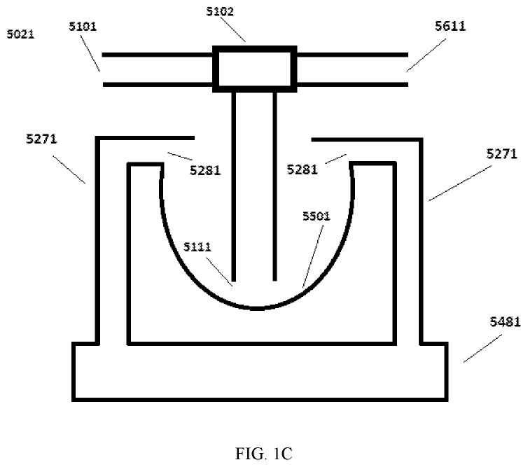

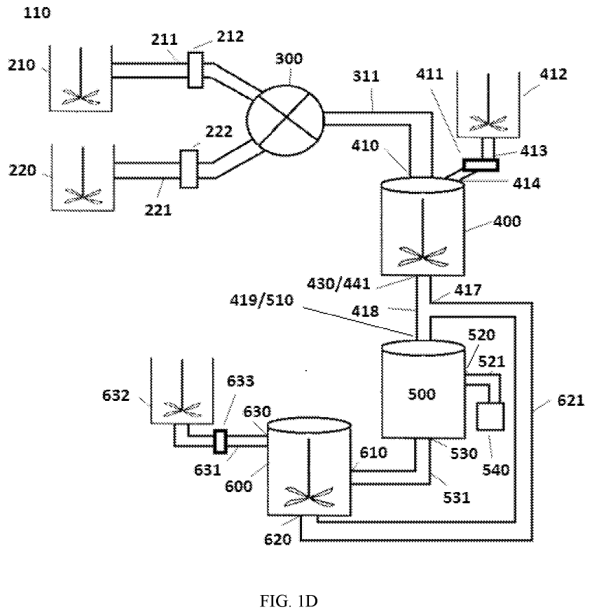

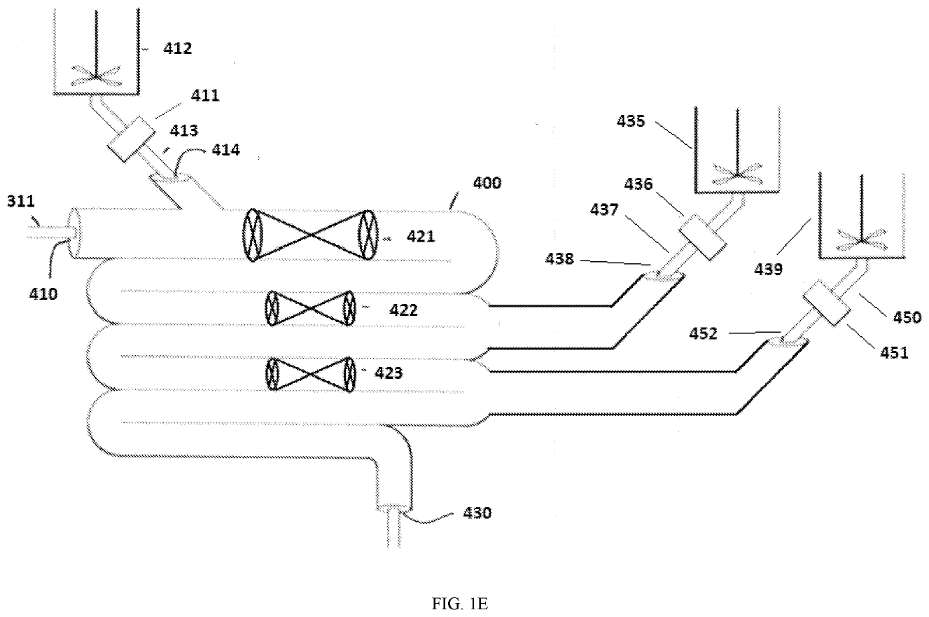

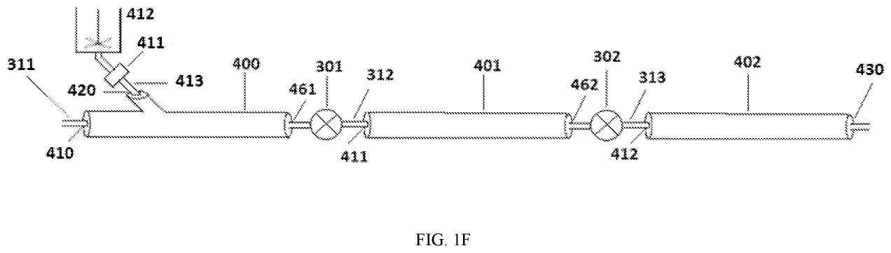

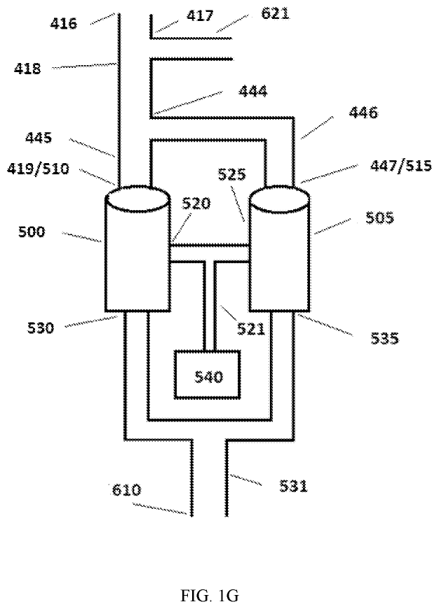

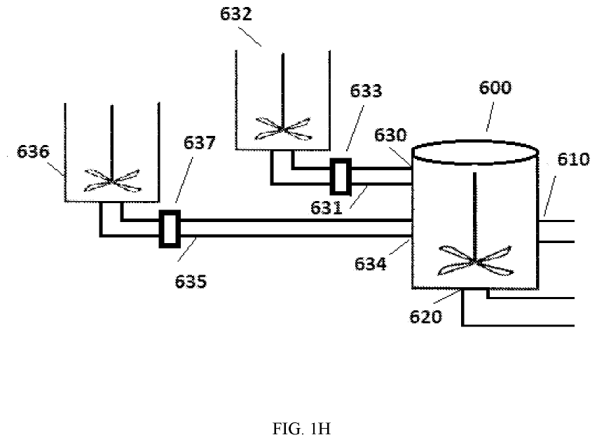

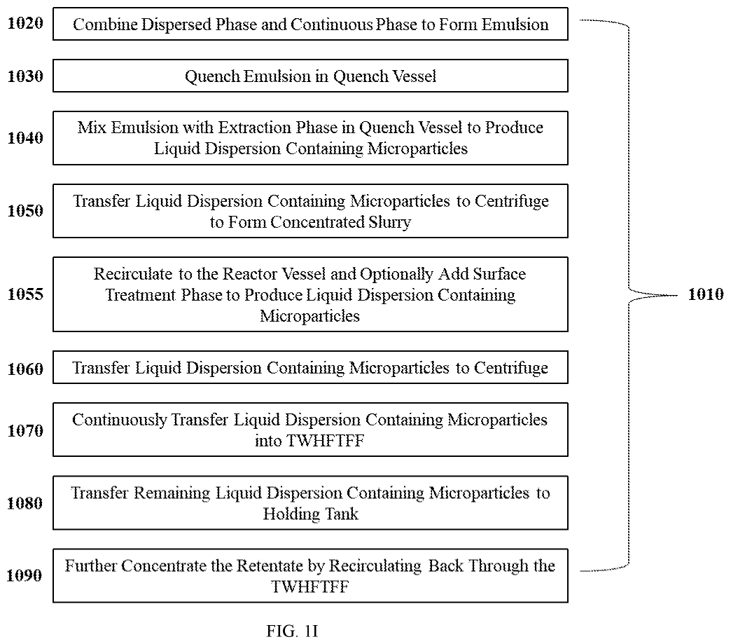

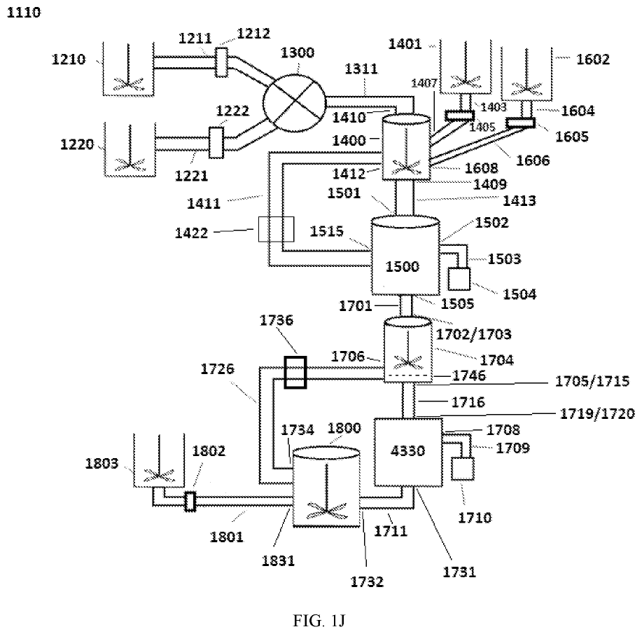

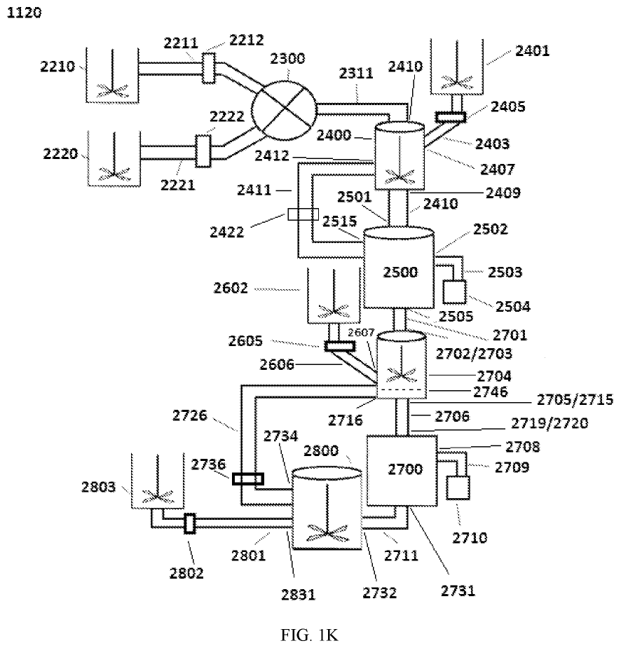

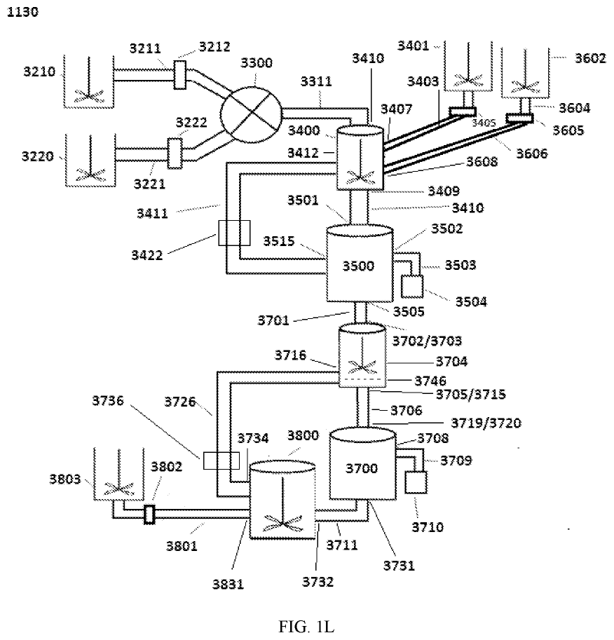

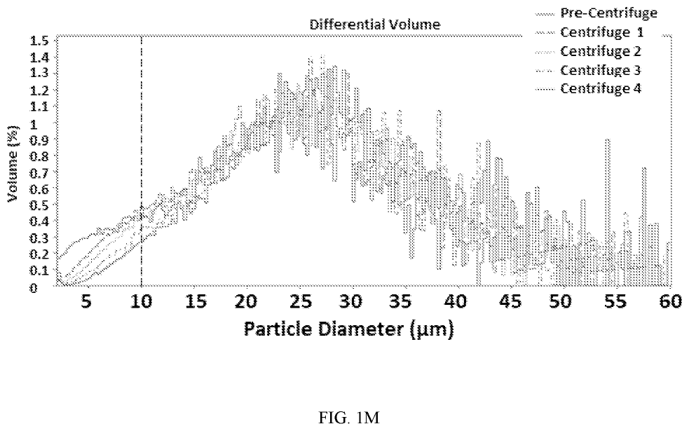

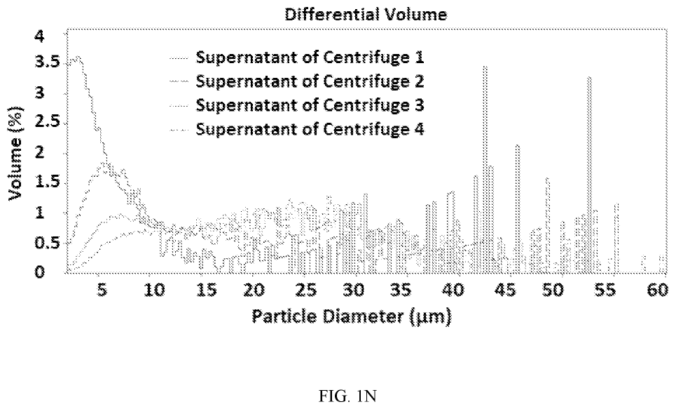

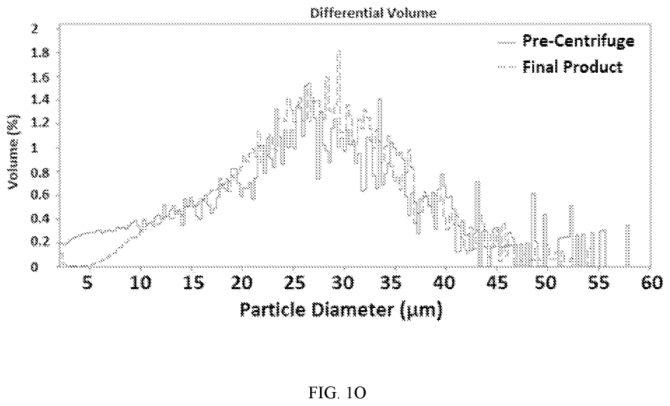

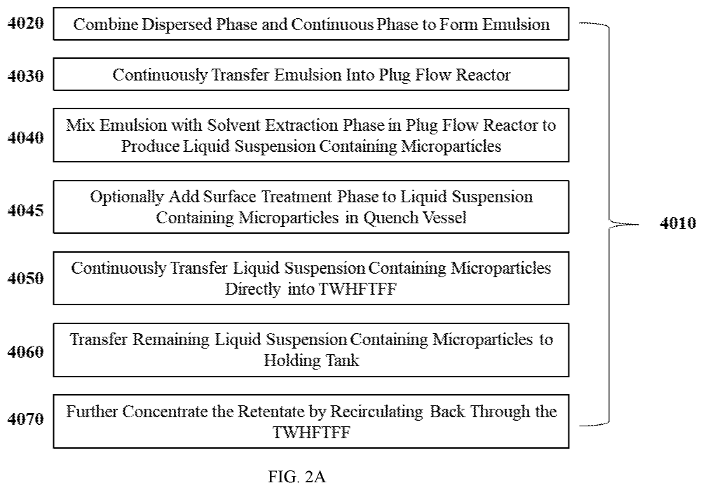

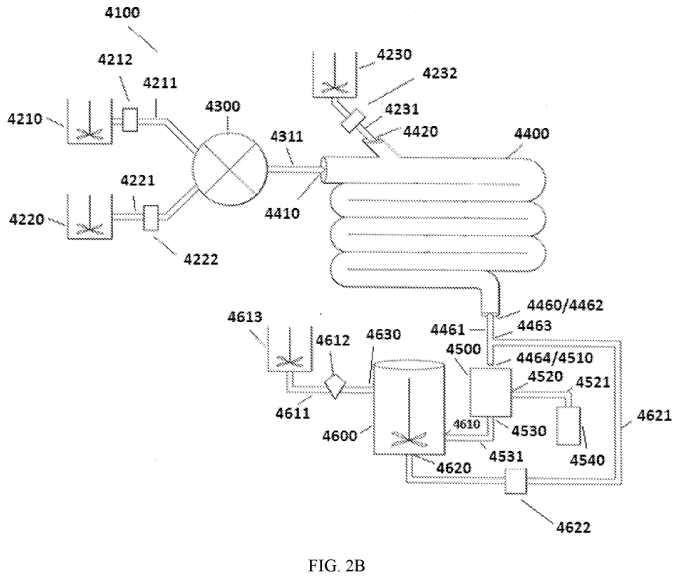

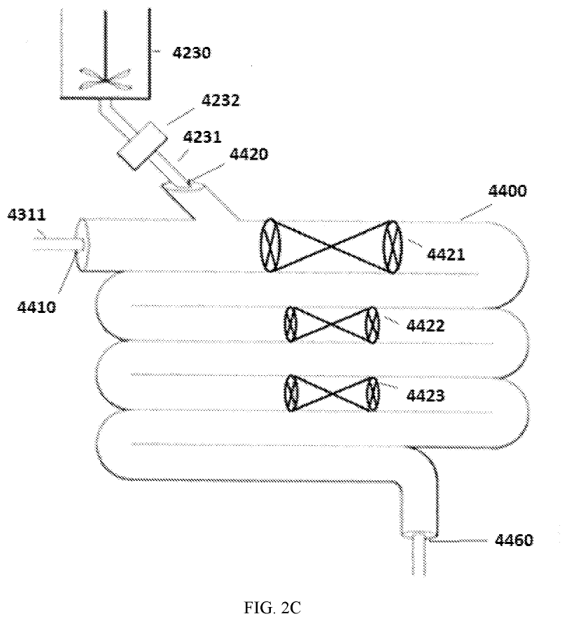

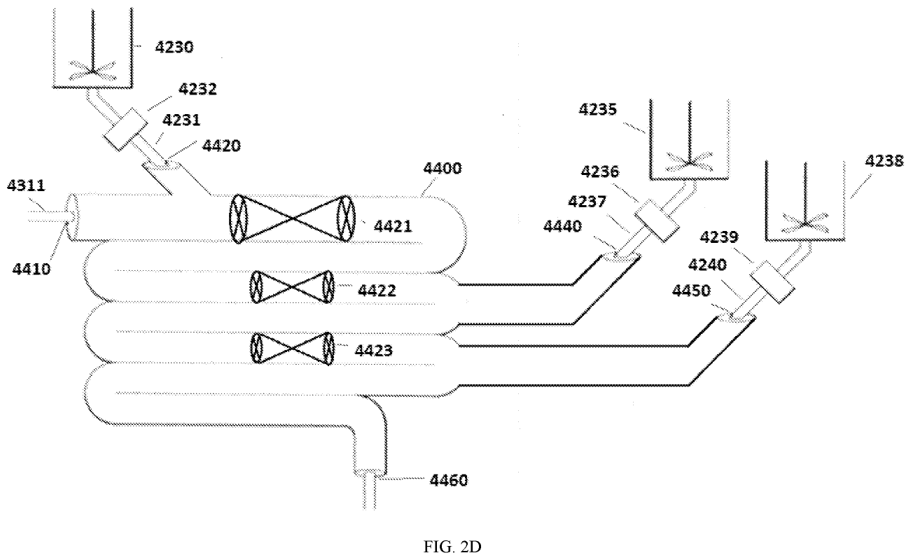

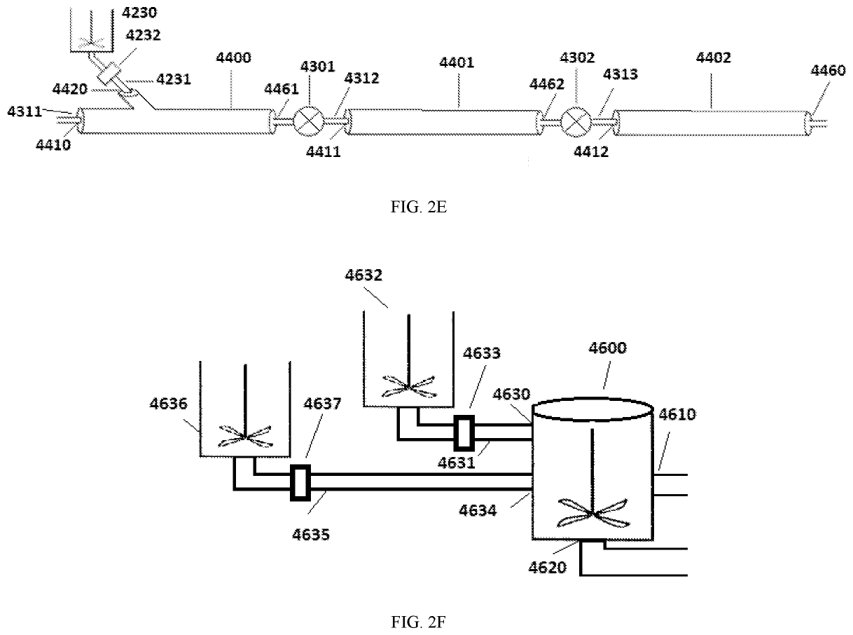

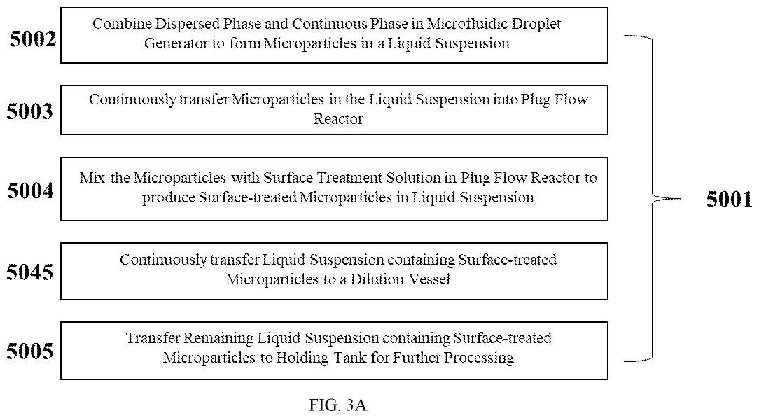

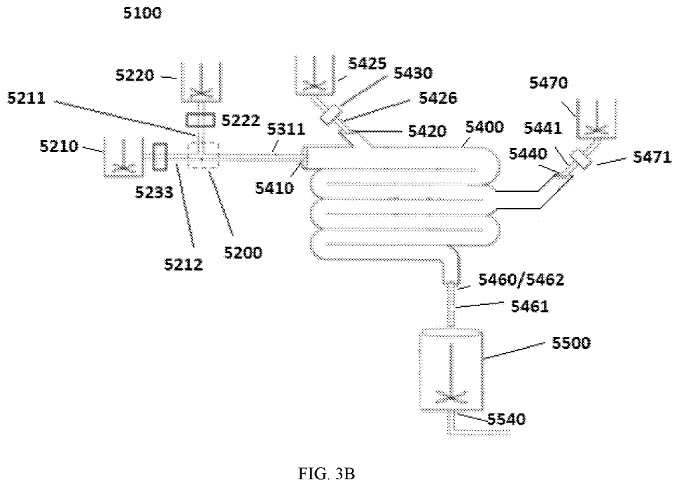

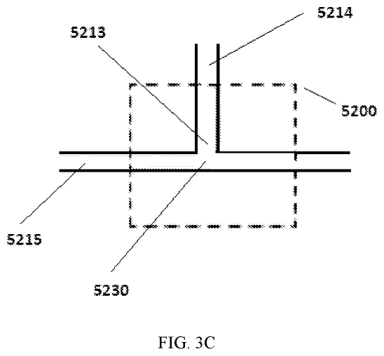

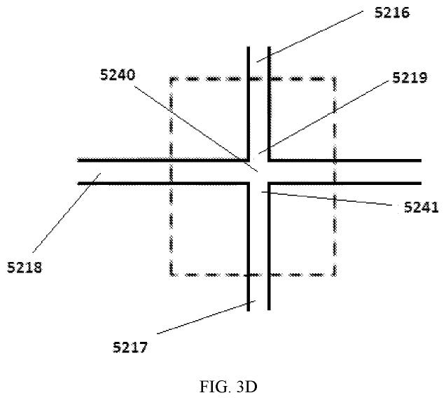

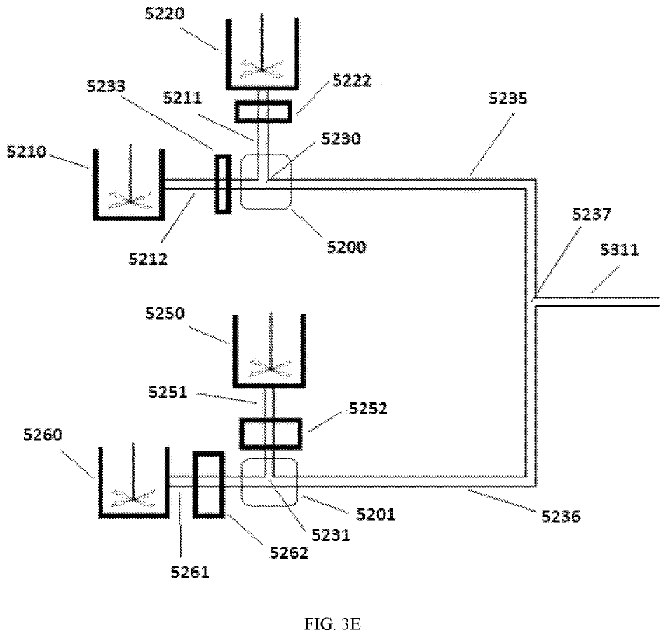

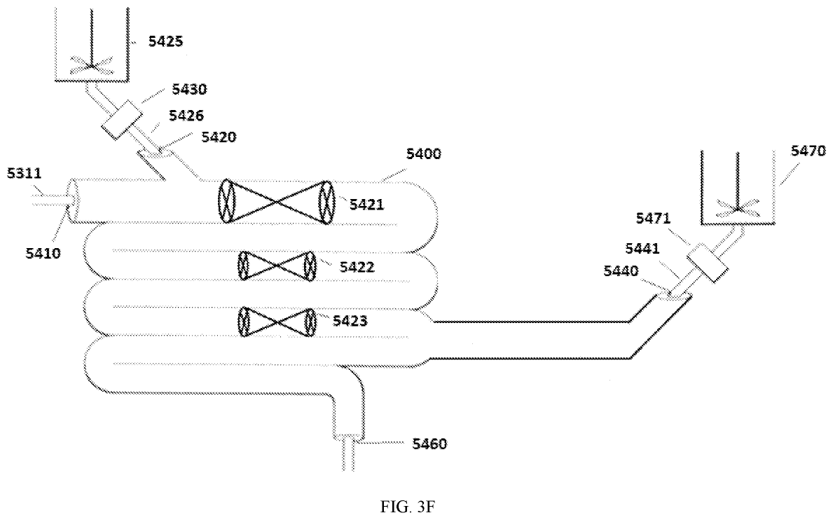

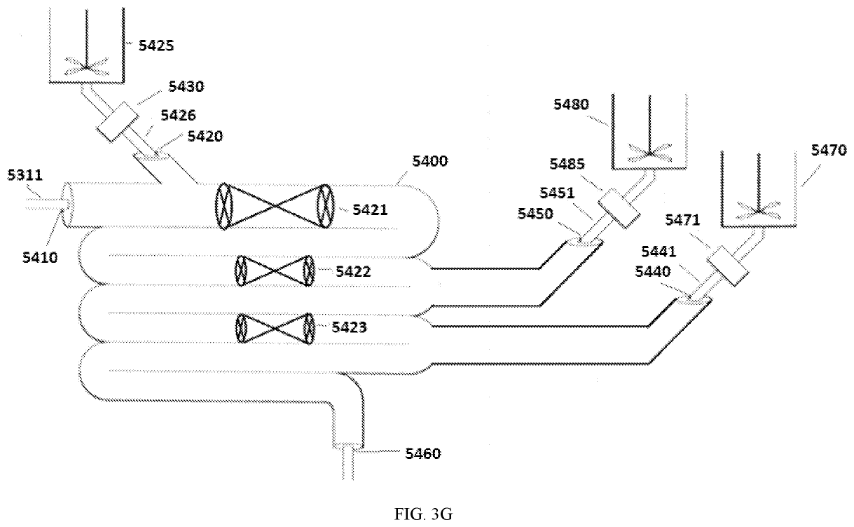

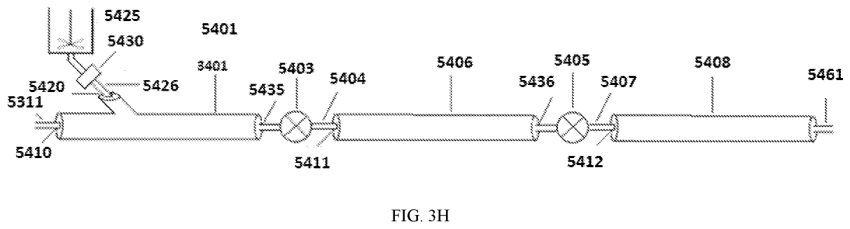

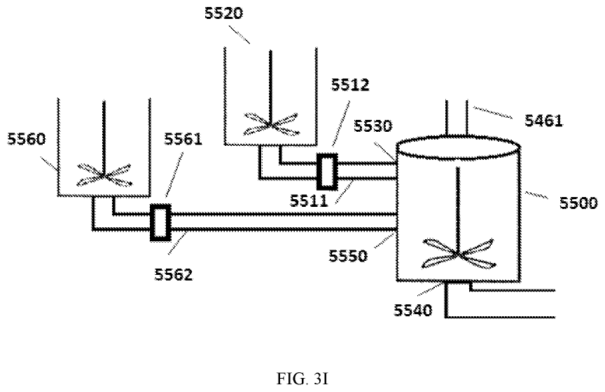

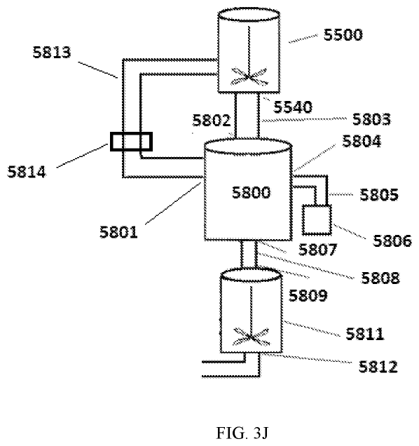

As shown in FIG. 1A, FIG. 1I, FIG. 2A, and FIG. 3A, processes for the production of drug-loaded microparticles are provided. In one aspect of the present invention, the production of microparticles involves the use of centrifugation in combination with a plug flow reactor (FIG. 1A) or a macro-filtration device such as a thick wall hollow fiber tangential flow filter (TWHFTFF (FIG. 1I). In an alternative aspect of the present invention, the production of microparticles utilizes a tangential flow filter (TFF) in combination with a plug flow reactor (FIG. 2A). In an alternative aspect of the present invention, the production of microparticles involves the use of a microfluidic droplet generator in combination with a centrifuge, a plug flow reactor, or a macro-filtration device such as a thick wall hollow fiber tangential flow filter (TWHFTFF) (FIG. 3A).

The microparticles may be biodegradable or non-biodegradable and include one or more active agents. The microparticles may be, for example, a nanoparticle, microsphere, nanosphere, microcapsule, nanocapsule, or particles, in general. Microparticles may be, for example, particles having a variety of internal structure and organizations including homogeneous matrices such as microspheres (and nanospheres) or heterogeneous core-shell matrices (such as microcapsules and nanocapsules), porous particles, multi-layer particles, among others. The microparticles may have mean by volume sizes in the range of at least about 10, 50, or 100 nanometers (nm) to about 100 micrometers (μm). In some embodiments, the microparticles have mean by volume sizes that are not greater than about 40 μm diameter. In certain embodiments, the microparticles have mean by volume sizes that are between about 20 to 40 μm, 10 to 30 μm, 20 to 30 μm, or 25 to 30 μm diameter. In certain embodiments, the microparticles have mean by volume sizes that are not greater than about 20, 25, 26, 27, 28, 29, 30, 35 or 40 μm diameter.

Preferably, the microparticles produced are biodegradable such that upon administration to a subject, for example a human or animal, such as a mammal, the microparticles gradually degrade over time, releasing the active agent. For example, the microparticle, once administered to the subject, can degrade over a period, for example over a period of days or months. The time interval can be from about less than one day to about 6 months or longer. In some embodiments, the microparticle releases the drug for at least one month, two months, three months, four months, five months, six months, seven months, eight, nine, ten, eleven, or twelve months. In certain instances, the polymer can degrade in longer time intervals, up to 2 years or longer, including, for example, from about 1 month to about 2 years, or about 3 months to 1 year, or 6 months to one year.

Continuous or Parallel Centrifugation

In one aspect of the present invention, provided herein is a process of producing drug-loaded microparticles in a continuous process which includes: a) continuously forming an emulsion comprising a dispersed phase and a continuous phase in a mixer, wherein the dispersed phase comprises a drug, a polymer, and at least one solvent; b) directly feeding the emulsion into a quench vessel, whereupon entering the quench vessel the emulsion is mixed with an extraction phase to form a liquid dispersion, wherein a portion of the solvent is extracted into the extraction phase and microparticles are formed; c) continuously feeding the liquid dispersion from the quench vessel into a parallel bank of centrifuges via an outlet from the quench vessel, wherein a portion of the liquid dispersion containing solvent and microparticles below a specified size threshold are removed with a waste solvent liquid and remaining microparticles above the specified size threshold are isolated as a concentrated slurry; and d) transferring the concentrated slurry from the centrifuge to a holding tank for further processing, if desired. In some embodiments, the liquid dispersion from the outlet of the quench vessel is diverted to a first centrifuge in a parallel bank of two or more centrifuges. After a set centrifugation time, the liquid dispersion from the outlet of the quench vessel is diverted into a one or more additional centrifuges instead of the first centrifuge. In some embodiments, the concentrated slurry is optionally rinsed with a wash phase while residing in the centrifuge. In some embodiments, the concentrated slurry present within the first centrifuge is optionally rinsed with a wash phase while the liquid dispersion is being diverted to one or more additional centrifuges within the parallel bank. In another embodiment, the liquid dispersion from the quench vessel is run through two or more centrifuges in a parallel bank of centrifuges operating simultaneously. In some embodiments, the concentrated slurry in the holding tank is optionally diluted with a wash phase and returned to the parallel bank of centrifuges for additional processing one or more times, for example, two, three, or four times. In some embodiments, the quench vessel is a plug flow reactor.

In one aspect of the present invention, provided herein is a process of producing drug-loaded microparticles in a continuous process which includes: a) continuously forming an emulsion comprising a dispersed phase and a continuous phase in a mixer, wherein the dispersed phase comprises a drug, a polymer, and at least one solvent; b) directly feeding the emulsion into a quench vessel, whereupon entering the quench vessel the emulsion is mixed with an extraction phase to form a liquid dispersion, whereupon a portion of the solvent is extracted into the extraction phase and microparticles are formed; c) continuously feeding the liquid dispersion from the quench vessel into a continuous liquid centrifuge via an outlet from the quench vessel, wherein a portion of the liquid dispersion containing solvent and microparticles below a specified size threshold are removed with a waste solvent liquid and remaining microparticles above the specified size threshold are isolated as a concentrated slurry; and d) continuously transferring the concentrated slurry from the centrifuge to a holding tank for further processing, if desired. In some embodiments, the continuous liquid centrifuge is a solid bowl centrifuge. In another embodiment, the continuous liquid centrifuge is a conical plate centrifuge. In some embodiments, the concentrated slurry is optionally rinsed with a wash phase while residing in the centrifuge. In some embodiments, the concentrated slurry in the holding tank is optionally diluted with a wash phase and returned to the continuous liquid centrifuge for additional processing. In some embodiments, the quench vessel is a plug flow reactor.

In one aspect of the embodiments herein, a surface treatment phase may be optionally added to the liquid dispersion of microparticles while present within the quench vessel. The surface treatment is typically added to facilitate aggregation of the formed microparticles when used in their intended application. In another aspect, a surface treatment phase may be optionally added to the concentrated slurry of microparticles when present within the centrifuge. In yet another aspect of the present invention, a surface treatment phase may be optionally added to the concentrated slurry of microparticles when present within the holding tank.

Various types of centrifuges may be used in any embodiments of the present invention. In some embodiments, the centrifuge is a filtration centrifuge. In some embodiments, the filtration centrifuge is selected from a conveyer discharge centrifuge, a pusher centrifuge, a peeler centrifuge, an inverting filter centrifuge, a sliding discharge centrifuge, and a pendulum centrifuge fitted with a perforated drum. In another embodiment, the centrifuge is a sedimentation centrifuge. In some embodiments, the sedimentation centrifuge is selected from a pendulum centrifuge fitted with a solid drum, a solid bowl centrifuge, a conical plate centrifuge, a tubular centrifuge, and a decanter centrifuge. In some embodiments, the centrifuge is an overflow centrifuge that allows continual removal of supernatant from the added liquid dispersion.

Upon reaching the holding tank as provided for in the above embodiments, the microparticles can be further processed, for example by continuous recirculation from the holding tank through one or more centrifuges to further remove solvent and microparticles of undesirable size. In some embodiments, the holding tank is pre-filled with a wash phase. In some embodiments, additional extraction phase is simultaneously added to the holding tank upon transfer of the concentrated slurry. In some embodiments, the holding tank is pre-filled with a wash phase, and, as the concentrated slurry enters the holding tank, additional wash phase is also continuously added. In certain embodiments, sufficient wash phase is added to the concentrated slurry in the centrifuge so that additional wash phase is not required during the remainder of the process, for example, upon entry into the holding tank. In some embodiments, one or more additional washes of the microparticles or one or more additional formulation steps may be performed on the concentrated slurry in the holding tank.

By using either a parallel bank of centrifuges or a continuous liquid centrifuge, residence time of the microparticles with extraction phase can be more tightly controlled. Thus, desirable microparticle drug elution characteristics can be derived and maintained by the high rate supernatant removal provided by the centrifuge and the subsequent further dilution of solvent through the exposure of the microparticles to further extraction phase in the holding tank. Because the process provides for a higher throughput due to the higher rate of supernatant removal, and thus a quicker processing time, the formed microparticles are less susceptible to further drug elution due to residual solvent presence and/or, in the case of highly hydrophilic drugs, extended residence in the extraction solvent.

In one aspect of the present invention, provided herein is a system and apparatus for producing and processing microparticles continuously comprising: a) a mixer suitable for receiving and combining a dispersed phase and continuous phase to form an emulsion; b) a quench vessel in direct fluid communication with the mixer via a first conduit, the quench vessel containing a first inlet for receiving the emulsion, a second inlet proximate to the first inlet for receiving an extraction phase, and an outlet; c) a continuous liquid centrifuge having an inlet in direct fluid communication with the outlet of the quench vessel by a second conduit, a first outlet, and a second outlet, wherein the first outlet of the centrifuge is capable of removing supernatant and the second outlet is capable of removing the concentrated slurry of microparticles, and the second conduit has a first inlet connected to the quench vessel and a second inlet distal from the first inlet; and d) a holding tank which is capable of receiving the concentrated slurry of microparticles from the centrifuge, wherein the holding tank has a first inlet in direct fluid communication via a third conduit with the second outlet of the centrifuge, and a first outlet, wherein the first outlet of the holding tank is in direct fluid communication via a fourth conduit with the second inlet of the second conduit.

In another aspect of the present invention, provided herein is an apparatus for producing and processing microparticles continuously comprising: a) a mixer; b) a quench vessel in direct fluid communication with the mixer; c) a continuous centrifuge in direct fluid communication with the quench vessel; d) a holding tank in direct fluid communication with the continuous centrifuge; and optionally e) a recirculating loop between the holding tank and the centrifuge.

In another aspect of the present invention, provided herein is an apparatus for producing and processing microparticles continuously comprising: a) a mixer; b) a quench vessel in direct fluid communication with the mixer; c) a continuous centrifuge in direct fluid communication with the quench vessel; d) a holding tank in direct fluid communication with the continuous centrifuge; and optionally e) a recirculating loop between the quench vessel and the centrifuge.

In another aspect of the present invention, provided herein is an apparatus for continuously producing and processing microparticles comprising: a) a mixer; b) a quench vessel in direct fluid communication with the mixer; c) a parallel bank of centrifuges in direct fluid communication with the quench vessel; d) a receiving vessel in direct fluid communication with the parallel bank of centrifuges; and optionally e) a recirculating loop between the receiving vessel and the centrifuge.

In another aspect of the present invention, provided herein is an apparatus for continuously producing and processing microparticles comprising: a) a mixer; b) a quench vessel in direct fluid communication with the mixer; c) a continuous centrifuge in direct fluid communication with the quench vessel; d) a receiving vessel in direct fluid communication with the continuous centrifuge; and optionally e) a recirculating loop between the quench vessel and the continuous centrifuge.

In another aspect of the present invention, provided herein is a system and apparatus for producing and processing microparticles continuously comprising: a) a mixer suitable for receiving and combining a dispersed phase and continuous phase to form an emulsion; b) a quench vessel in direct fluid communication with the mixer via a first conduit, the quench vessel containing a first inlet for receiving the emulsion, a second inlet proximate to the first inlet for receiving an extraction phase, and an outlet; c) a parallel bank of two or more centrifuges, each centrifuge having an inlet in direct fluid communication to the outlet of the quench vessel by a second conduit, a first outlet, and a second outlet, wherein the first outlet of the centrifuge is capable of removing supernatant and the second outlet is capable of removing the concentrated slurry of microparticles, and the second conduit has a first inlet connected to the quench vessel and a second inlet distal from the first inlet; and d) a holding tank which is capable of receiving the concentrated slurry of microparticles from the centrifuge, wherein the holding tank has a first inlet in direct fluid communication via a third conduit with the second outlet of the centrifuge, and a first outlet, wherein the first outlet of the holding tank is in direct fluid communication via a fourth conduit with the second inlet of the second conduit.

In another aspect of the present invention, provided herein is an apparatus for producing and processing microparticles continuously comprising: a) a mixer; b) a quench vessel in direct fluid communication with the mixer; c) a parallel bank of centrifuges in direct fluid communication with the quench vessel; d) a holding tank in direct fluid communication with the continuous centrifuge; and optionally e) a recirculating loop between the holding tank and the centrifuge.

In another aspect of the present invention, provided herein is an apparatus for producing and processing microparticles continuously comprising: a) a mixer; b) a quench vessel in direct fluid communication with the mixer; c) a parallel bank of centrifuges in direct fluid communication with the quench vessel; d) a holding tank in direct fluid communication with the continuous centrifuge; and optionally e) a recirculating loop between the quench vessel and the centrifuge.

Centrifugation in Combination with a Plug Flow Reactor

Referring to FIG. 1A, in an embodiment, a process for producing microparticles 10 is provided wherein a dispersed phase and continuous phase are fed into a mixer to form an emulsion 20, which is subsequently transferred into a quench vessel 30. In some embodiments, the quench vessel is a batch reactor, filter reactor system, or a stir tank. In another embodiment, the quench vessel is a tubular reactor.

In some embodiments of any of the aspects described herein, the quench vessel is a plug flow reactor. Plug flow reactors, also referred to as continuous tubular reactors or piston flow reactors, are known in the art and provide for interactions of materials in continuous, flowing systems of cylindrical geometry. The use of a plug flow reactor allows for the same residence time for all fluid elements in the tube. Comparatively, the use of holding vessels or stir tanks for mixing and solvent removal leads to different residence time and uneven mixing. Complete radial mixing as present in plug flow eliminates mass gradients of reactants and allows contact between reactants, often leading to faster reaction times and more controlled conditions. Additionally, complete radial mixing allow for uniform dispersion and conveyance of solids along the tube of the reactor, providing more consistent microparticle size formation. The traversal and continuous mixing of the liquid dispersion as it traverses the plug flow reactor further assists in continuous solvent removal and microparticle hardening. By using a plug flow reactor, residence time of the microparticle in the liquid dispersion can be tightly controlled, allowing for the consistent production of microparticles.

In some embodiments, the plug flow reactor contains one or more apparatuses within the cylinder, for example a mixer that provides for additional mixing. For example, StaMixCo has developed a static mixer system that allows for plug flow by inducing radial mixing with a series of static grids along the tube.

In some embodiments, the plug flow reactor is a continuous oscillatory baffled reactor (COBR). In general, the continuous oscillatory baffled reactor consists of a tube fitted with equally spaced baffles presented transversely to an oscillatory flow. The baffles disrupt the boundary layer at the tube wall, whilst oscillation results in improved mixing through the formation of vortices. By incorporating a series of equally spaced baffles along the tube, eddies are created when liquid is pushed along the tube, allowing for sufficient radial mixing.

In some embodiment, one or more further extraction phases are added into the plug flow reactor distally from the initial addition. The incorporation of additional extraction phases can further assist in solvent extraction, resulting in a full extraction prior to the exiting of the liquid dispersion from the plug flow reactor.

Referring again to FIG. 1A, in some embodiments, process 10 includes mixing extraction phase 40 with the emulsion. The emulsion formed in 20 is transferred into a quench vessel 30, wherein it is further mixed with an extraction phase 40. The extraction phase comprises a single solvent for extracting the solvent or solvents used to formulate the dispersed phase. In some embodiments, the extraction phase may comprise two or more co-solvents for extracting the solvent or solvents used to formulate the dispersed phase. Different polymer non-solvents (i.e., extraction phase), mixtures of solvents and polymer non-solvents and/or reactants for surface modification/conjugation may be used during the extraction process to produce different extraction rates, microparticle morphology, surface modification and polymorphs of crystalline drugs and/or polymers. In one aspect, the extraction phase comprises water or a polyvinyl alcohol solution. In some embodiments, the extraction phase comprises primarily or substantially water. The actual ratios of extraction phase to emulsion will depend upon the desired product, the polymer, the drug, the solvents, etc., and can be determined empirically by those of ordinary skill in the art. For example, the ratio of extraction phase to emulsion phase is 2:1. This translates into a flow rate of about 4000 mL/min for the extraction phase when the flow rate of the emulsion upon entry into the plug flow reactor is about 2000 mL/min. A typical plug flow reactor as used in the present invention can be any size that achieves the desired result. In some embodiments, it is about 0.5 inches in diameter and can typically range from, for example about 0.5 meters to for example, about 30 meters in length depending on the desired residence time. In some embodiments, the plug flow reactor length is about 0.5 meters to about 30 meters, about 3 meters to about 27 meters, about 5 meters to about 25 meters, about 10 meters to about 20 meters, or about 15 meters to about 18 meters. Residence times within the plug flow reactor can be set to any time that achieves the desired results. In some embodiments, it can range from about 10 seconds to about 30 minutes depending on the desired application. In some embodiments, the residence time is about up 10 seconds, about up 20 seconds, about up 1 minute, about up 2 minutes, about up 5 minutes, about up 10 minutes, about up 20 minutes, about up 25 minutes, or about up 30 minutes. In some embodiments, only one extraction phase is introduced into a plug flow reactor with a length of about 0.5 meters and have a residence time from about 10 to 20 seconds up to about 2.5 minutes. In an additional embodiment, extraction phase and surface treatment solution are introduced into a plug flow reactor with a length of about 30 meters and a residence time between about 25 and 35 minutes.

Referring again to FIG. 1A, as the emulsion is fed into the quench vessel 30, the extraction phase is introduced into the quench vessel and the emulsion and extraction phase are continually mixed 40. Upon mixing, the solvent from the dispersed phase is extracted into the extraction phase and microparticles are formed in a liquid dispersion.

In some embodiments, one or more further solvent extraction phases are added into the quench vessel distally from the initial addition. The incorporation of additional solvent extraction phases can further assist in solvent extraction, resulting in a full extraction prior to the exiting of the liquid dispersion from the quench vessel.

Referring again to FIG. 1A, in some embodiments, process 10 further includes one or more surface treatment phases optionally added 45 into the quench vessel distally from the initial addition of extraction phase.

Following mixing of the emulsion with the extraction phase in the quench vessel to form a liquid dispersion containing microparticles 40 and an optional surface treatment 45, the liquid dispersion is transferred from the quench vessel to either a continuous liquid centrifuge or a parallel bank of centrifuges to form a concentrated slurry 50. In certain embodiments, the quench vessel and centrifuge are arranged in tandem, that is, in direct fluid communication with each other. In some embodiments, the quench vessel and centrifuge are directly connected through a conduit which allows for the liquid dispersion to exit the quench vessel and enter the centrifuge. The types of centrifuges appropriate for this application are known to those having skill in the art. The rotational speed of the centrifuge will typically determine the size range for the microparticles that are isolated therein. In typical embodiments, the rotational speed is from about 2000 rpm to about 3000 rpm.

Centrifugation Techniques

In some embodiments, the centrifuge is a filtration centrifuge. A filtration centrifuge contains an inner drum that is perforated and fitted with a filter, for example a cloth or wire mesh, with an appropriate pore size to allow removal of solvent and microparticles of undesired size. Upon induction of centrifugal force, the liquid dispersion flows from the inside to the outside through the filter and the perforated drum. The concentrated slurry of microparticles is then collected on the filter and transferred to the holding tank. The pore size can be chosen to achieve the desired results. In some embodiments, the pore size of the filter is between about 1 μm and 100 μm. In some embodiments, the pore size of the filter is at least about 1 μm and 80 μm. In some embodiments, the pore size of the filter is between about 1 μm and 25 μm. In some embodiments, the pore size of the filter is between about 5 μm and 10 μm. In some embodiments, the pore size of the filter is between about 2 μm and 5 μm. In some embodiments, the pore size of the filter is between about 6 μm and 8 μm. By incorporating a larger pore size, the resultant concentration of microparticles is more uniform, allowing for a reduction in the number of additional processing steps necessary to derive a microparticle product of desired size. The use of a filter centrifuge allows continuous addition of the liquid dispersion to the centrifuge. Non-limiting examples of filter centrifuges include conveyer discharge centrifuges, pusher centrifuges, peeler centrifuges, inverting filter centrifuges, sliding discharge centrifuges, and pendulum centrifuges fitted with a perforated drum.

In another embodiment, the centrifuge is a sedimentation centrifuge. A sedimentation centrifuge contains a solid inner drum without perforation. Upon induction of centrifugal force, the microparticles contained within the liquid dispersion deposit on the wall of the solid inner drum. The supernatant can be subsequently removed to provide the concentrated slurry of microparticles. The supernatant can be removed once sedimentation of the microparticles is complete or can be removed continuously during rotation. Non-limiting examples of sedimentation centrifuges include a pendulum centrifuge fitted with a solid drum, separator or continuous liquid centrifuges such as solid bowl centrifuges or conical plate centrifuges, tubular centrifuges, and decanter centrifuges. In some embodiments, the sedimentation centrifuge is an overflow centrifuge. An overflow centrifuge contains a liquid discharge outlet that drains the supernatant away during application of centrifugal force, allowing constant addition of the liquid dispersion containing the microparticles to the centrifuge. The overflow centrifuge may also contain a solid discharge outlet in addition to the liquid discharge outlet to allow continual removal of the concentrated slurry from the centrifuge to the holding tank during processing.

In some embodiments, the liquid dispersion from the outlet of the quench vessel is diverted to a first centrifuge in a parallel bank of two or more centrifuges. After a set centrifugation time, the liquid dispersion from the outlet of the quench vessel is diverted into one or more additional centrifuges instead of the first centrifuge. This may be required, for example, upon saturation of the centrifuge barrel with concentrated slurry in a first centrifuge in order to maintain sufficient isolation of the microparticles as a concentrated slurry. In some embodiments, the conduit from the quench vessel to the first centrifuge contains a valve, for example a T valve that allows for diversion of the liquid dispersion from the quench vessel to a second centrifuge instead of the first centrifuge. In some embodiments, the liquid dispersion is instead divided among two or more parallel centrifuges that are running concurrently. This may be accomplished by splitting the conduit from the quench vessel into several conduit lines among two or more parallel centrifuges. In some embodiments, the concentrated slurry present within the first centrifuge is optionally rinsed with a wash phase while the liquid dispersion is being diverted to one or more additional centrifuges within the parallel bank. The wash phase may be of the same composition as the extraction phase used prior or may be a different solvent composition such as those described for the dispersed phase or the continuous phase as deemed appropriate for the particular application. In some embodiments, the wash phase is water.

FIG. 1B provides a non-limiting example of a continuous liquid centrifuge, in particular a solid bowl centrifuge, that may be used in the present invention. The centrifuge 5010 comprises an inner rotating drum 5600 arranged horizontally. The liquid dispersion enters the centrifuge 5010 via centrifuge inlet 5160 and exits dispersion outlet 5110 to be splayed on the inside wall of the rotating inner drum 5600. The deposition of microparticles sediments on the inner surface of the rotating inner drum 5600 due to centrifugal force. The centrifuge also contains outlet 5270 for the supernatant and outlet 5300 for the concentrated slurry that is formed. As more liquid dispersion is added to the centrifuge, supernatant overflows from 5510 into outlet 5270, where it is directed by conduit 5280 to a waste tank. The concentrated slurry that is formed is removed as its sedimentation builds up via outlet 5300 into conduit 5310 that leads to the holding tank.

FIG. 1C provides an additional non-limiting example of a centrifuge that may be used in the present invention. The centrifuge 5021 comprises an inner rotating drum 5501 arranged vertically. The liquid dispersion enters the centrifuge 5021 via centrifuge inlet 5101 and exits dispersion outlet 5111 to be splayed on the inside wall of the rotating inner drum 5501. The deposition of microparticles sediments on the inner surface of the rotating inner drum 5501 due to centrifugal force. As the level of supernatant increases within the rotating inner drum 5501, it overflows into outlets 5281 and is drawn through conduits 5271 into a waste tank 5481. To remove the concentrated slurry from the rotating inner drum 5501, a wash phase is added via centrifuge inlet 5101 and dispersed via outlet 5111 to bring up the microparticles again as a liquid dispersion. A directional valve 5102 is then switched from directing flow into the centrifuge via inlet 5101 to removing the newly formed liquid dispersion via dispersion outlet 5111 into centrifuge outlet 5611 which removes the dispersion into the receiving tank. This type of centrifuge is an example of one that would be appropriate for use in a parallel bank of centrifuges.

An exemplary centrifuge is the Viafuge® Pilot available from Pneumatic Scale Angelus. Referring again to FIG. 1A, in process 10, upon entry of the liquid dispersion containing microparticles into the centrifuge, a portion of the dispersion is removed as supernatant. The supernatant can be sent to waste or, in certain embodiments, recycled for further use. The concentrated slurry remaining within the centrifuge is subsequently transferred to a holding tank 60.

Referring again to FIG. 1A, in some embodiments, process 10 requires additional processing of the concentrated slurry 65 to obtain microparticles of sufficient purity once transferred to the holding tank. In some embodiments, the microparticles may be further purified by recirculating the concentrated slurry obtained in the holding tank back through the centrifuge. Further processing typically requires dilution of the concentrated slurry with a wash phase. In some embodiments, the holding tank may contain a wash phase. For example, the concentrated slurry exiting the centrifuge may be transferred to a holding tank containing a predetermined amount of wash phase. Alternatively, a wash phase may be added to the holding tank after transfer of the concentrated slurry. Additionally, the holding tank may include a starting amount of wash phase, and as recirculation occurs, an additional amount of wash phase is continuously added. If additional rinsing of the microparticles within the slurry is desired, the wash phase is typically added at the same flow rate as for supernatant removal in the centrifuge. If concentration of the microparticles within the slurry is instead desired, no wash phase is added upon recirculation. Alternatively, the microparticles within the slurry may also instead be optionally treated with a surface treatment solution during recirculation either in addition to or in replacement of the wash phase.

Accordingly, the holding tank includes an outlet in fluid communication with a conduit from the quench vessel to the centrifuge such that the concentrated slurry diluted with wash phase can be sent from the holding tank back through the centrifuge. The recirculation may occur following the completion of production of the microparticles. For example, following completion of microparticle formation, all of the concentrated slurry containing the microparticles is collected in the holding tank, diluted with a wash phase, and subsequently recirculated back through the centrifuge for further concentration and washing. Alternatively, recirculation through the centrifuge can be performed continuously, for example, as a continuous process such that as soon as the concentrated slurry is received in the holding tank, it is diluted with a wash phase and then recirculated back through the centrifuge as the microparticle batch processing continues. Also provided herein is a system, system components, and an apparatus for producing and processing microparticles as described herein. FIG. 1D represents one non-limiting embodiment of a system 110 for producing microparticles according to the processes described herein. In some embodiments, the system incorporates one or more of the system elements described in FIG. 1A.

Referring to FIG. 1D, in some embodiments, system 110 includes a dispersed phase holding tank 210 and a continuous phase holding tank 220. The dispersed phase holding tank 210 includes at least one outlet, and is capable of mixing one or more active agents, one or more solvents for the active agent, one or more polymers, and one or more solvents for the polymer to form a dispersed phase. Likewise, the continuous phase holding tank 220 contains at least one outlet. The dispersed phase holding tank 210 is in fluid communication with a mixer 300 via conduit 211. Likewise, the continuous phase holding tank 220 is in fluid communication with mixer 300 via conduit 221. Conduit 211 and 221 may further include a filtering device 212 and 222, respectively, for sterilizing the phases before entry into mixer 300. In some embodiments, the filtering device is any suitable filter for use to sterilize the phases, for example a PVDF capsule filter.

Mixer 300 can be any suitable mixer for mixing the dispersed phase with the continuous phase to form either an emulsion or microparticles in a liquid dispersion. In some embodiments, mixer 300 is an in-line high shear mixer. The mixer 300 receives the dispersed phase and the continuous phase and mixes the two phases. In some embodiments, the mixer 300 includes at least one outlet for transferring the formed emulsion or microparticles in liquid dispersion to a quench vessel 400. The formed emulsion or microparticles contained in the liquid dispersion are transferred from the mixer 300 to quench vessel 400 via conduit 311. Quench vessel 400 includes inlet 410 for receiving the formed emulsion or microparticles in the liquid dispersion, and one or more additional inlets for receiving extraction phase. Referring to FIG. 1D, extraction phase holding tank 412 transfers extraction phase to the quench vessel inlet 414 via conduit 413. Conduit 413 may further include a suitable sterilization filter 411, for example as previously described, for filtering the extraction phase prior to entering the quench vessel 400.

In some embodiments, the quench vessel 400 as used in the system is a plug flow reactor 400. A non-limiting embodiment of a plug flow reactor as the quench vessel 400, optionally with one or more additional mixers is provided in FIG. 1E. Referring to FIG. 1E, the plug flow reactor 400 is connected to conduit 311 by inlet 410. The plug flow reactor 400 contains an additional inlet 414 that is connected to conduit 413 for receiving the extraction phase from the extraction phase holding tank 412. The plug flow reactor 400 additionally contains outlet 430 for transferring the liquid dispersion to the centrifuge. One or more additional mixers may be placed within the plug flow reactor to further assist in mixing the emulsion or microparticles in the liquid dispersion with the solvent extraction phase. For example, mixer 421 is placed distally from inlet 414, allowing additional mixture of the liquid dispersion with the solvent extraction phase. In certain embodiments, additional mixers can be placed distally from mixer 421, as illustrated by mixers 422 and 423.

The plug flow reactor may include additional inlets for receiving solvent extraction phase. For example, as illustrated in FIG. 1E, additional inlets may be included in the plug flow reactor 400. For example, additional solvent extraction phase holding tanks 435 and 439 can transfer additional solvent extraction phase in two different locations distally from initial solvent extraction phase inlet 414, for example, at inlets 438 and 452, respectively, via conduit 437 and 450. By introducing additional solvent extraction phase inlets proximate to a mixer, upon addition of the solvent extraction phase, the solvent extraction phase can be thoroughly mixed with the liquid dispersion as it traverses the plug flow reactor, providing additional solvent removal to take place. The additional solvent extraction addition conduit 437 and 450 may optionally contain a suitable sterilization filter 436 and 451, respectively, for example as previously described, for filtering the solvent extraction phase prior to entering the plug flow reactor 400.

In another embodiment, the plug flow reactor may comprise a series of plug flow reactors in direct fluid communication via a series of static mixers. For example, as illustrated in FIG. 1F, plug flow reactor 400 may alternatively be in direct fluid communication with static mixer 301 via outlet 461. The microparticle dispersion formed may flow out from static mixer 301 via conduit 312 to a second plug flow reactor 401 via inlet 411. Plug flow reactor 401 may be in direct fluid communication with static mixer 302 via outlet 462. The microparticle dispersion formed may flow out from static mixer 302 via conduit 313 to a third plug flow reactor 402 via inlet 412. The third plug flow filter 402 also has outlet 430 that is in direct fluid communication centrifuge 500.

Referring to FIG. 1D, the quench vessel 400 includes outlet 430 for transferring the liquid dispersion including microparticles from the quench vessel 400 to a centrifuge 500. The quench vessel is in direct fluid communication with centrifuge 500 via conduit 418. Conduit 418 includes a first inlet 441 connected to the quench vessel outlet 430 and a second inlet 417. Conduit 418 also includes outlet 419 connected to the centrifuge 500 at the centrifuge inlet 510. During processing, the liquid dispersion including microparticles is transferred from the quench vessel 400 and enters the centrifuge 500 via conduit 418. The centrifuge includes a first outlet 520 proximate to a second outlet 530. Upon entry into the centrifuge, supernatant is removed through outlet 520. In some embodiments, supernatant is transferred to a waste tank 540 through outlet 520. In some embodiments, the centrifuge is a continuous liquid centrifuge as shown in FIG. 1B, wherein outlet 419 of conduit 418 is in direct fluid communication with inlet 5160 of the continuous liquid centrifuge, the concentrated slurry outlet 5310 is in direct fluid communication with the conduit 531 that leads to holding tank 600, and the supernatant outlet 5280 is in direct fluid communication with conduit 521 that leads to waste tank 540. In another embodiment, the centrifuge is as shown in FIG. 1C, wherein outlet 4193 of conduit 418 is in direct fluid communication with the inlet 5101 of the centrifuge and the centrifuge outlet 5611 is in direct fluid communication with conduit 531 that leads to holding tank 600.

In another embodiment, the system includes a parallel bank of centrifuges. Referring to FIG. 1G, conduit 418 contains a first inlet 416 for the liquid dispersion from the quench vessel and a second inlet 417. Conduit 418 diverges at junction 444 into conduit 445 and 446 directed respectively to first centrifuge 500 and second centrifuge 505. In some embodiments, junction 444 contains a valve that selectively directs the liquid dispersion to either first centrifuge or second centrifuge 505 via conduit 445 and 446, respectively. The direction of flow for the liquid dispersion can be directed from the first centrifuge 500 to the second centrifuge 505, or vice versa, by adjusting the valve at junction 444. Conduit 445 is connected via outlet 419 to inlet 510 of first centrifuge 500, and conduit 446 is connected via outlet 447 to inlet 515 of second centrifuge 505. First centrifuge 500 also contains a first outlet 520 and a second outlet 530, and second centrifuge 505 contains a first outlet 525 and a second outlet 535. Supernatant is removed from first centrifuge 500 and second centrifuge 505 by outlets 520 and 525, respectively. Outlets 520 and 525 converge onto conduit 521 that transfers supernatant to waste tank 540. Outlets 530 and 535 remove the concentrated slurry from first centrifuge 500 and second centrifuge 505, respectively, and converge onto conduit 531 to transfer the concentrated slurry to the holding tank through holding tank inlet 610.

Referring to FIG. 1D, system 100 further includes a holding tank 600 in fluid communication with the centrifuge 500 via conduit 531. The concentrated slurry containing the microparticles exits the centrifuge 500 at outlet 530 and is transferred to holding tank 600 via conduit 531 through holding tank inlet 610. Holding tank 600 also includes outlet 620 and optionally one or more inlets. As illustrated in FIG. 1D, holding tank 600 includes additional inlet 630 for receiving a wash phase. In some embodiments, the wash phase is added to holding tank 600 from wash phase holding phase tank 632 via conduit 631. Conduit 631 may further comprise a filter, for example as previously described, for sterilizing the additional extraction phase prior to entry into holding tank 600.

Referring again to FIG. 1D, in one embodiment, holding tank 600 may alternatively include two inlets 630 and 634 that allow a wash phase and a surface treatment phase to be added either separately or simultaneously. As shown in FIG. 1H, wash phase is added to holding tank 600 from wash phase holding tank 632 via conduit 631 and surface treatment phase is added to holding tank 600 from surface treatment phase holding tank 636 via conduit 635. Conduits 631 and 635 may further comprise filters 633 and 637, respectively, for sterilizing the phases prior to entry into holding tank 600.

Referring again to FIG. 1D, in one embodiment, holding tank 600 is in further fluid communication with conduit 418 via conduit 621. Conduit 621 connects holding tank outlet 620 with second inlet 417 of conduit 418. Upon entry of the concentrated slurry into holding tank 600 and subsequent dilution with wash phase, the direct fluid connection with conduit 418 via conduit 621 allows the liquid dispersion to be recirculated through the centrifuge 500 as described above.

Continuous or Parallel Centrifugation in Combination with TWHFTFF In one aspect of the present invention, provided herein is a process of producing drug-loaded microparticles in a continuous process which includes: a) continuously forming an emulsion comprising a dispersed phase and a continuous phase in a mixer, wherein the dispersed phase comprises a drug, a polymer, and at least one solvent; b) directly feeding the emulsion into a quench vessel, whereupon entering the quench vessel the emulsion is mixed with an extraction phase to form a liquid dispersion, whereupon a portion of the solvent is extracted into the extraction phase and microparticles are formed; c) continuously feeding the liquid dispersion from the quench vessel into a continuous liquid centrifuge via an outlet from the quench vessel, wherein a portion of the liquid dispersion containing solvent and microparticles below a specified size threshold are removed with a waste solvent liquid and remaining microparticles above the specified size threshold are isolated as a concentrated slurry; and d) continuously recirculating the concentrated slurry from the continuous liquid centrifuge to the quench vessel, whereupon entering the quench vessel, the concentrated slurry is rinsed with water or mixed with surface treatment phase; e) continuously transferring the microparticles from the liquid centrifuge to a receiving vessel for further processing, if desired. In some embodiments, the continuous liquid centrifuge is a solid bowl centrifuge. In another embodiment, the continuous liquid centrifuge is a conical plate centrifuge. In some embodiments, the concentrated slurry is optionally rinsed with a wash phase while residing in the centrifuge. In some embodiments, the receiving vessel is connected to a thick wall hollow fiber tangential flow filter (TWHFTFF).

The process of producing drug-loaded microparticles in a continuous process includes a) continuously forming an emulsion comprising a dispersed phase and a continuous phase in a mixer, wherein the dispersed phase comprises a drug, a polymer, and at least one solvent; b) directly feeding the emulsion into a quench vessel, whereupon entering the quench vessel the emulsion is mixed with an extraction phase to form a liquid dispersion, whereupon a portion of the solvent is extracted into the extraction phase and microparticles are formed; c) continuously feeding the liquid dispersion from the quench vessel into a continuous liquid centrifuge via an outlet from the quench vessel, wherein a portion of the liquid dispersion containing solvent and microparticles below a specified size threshold are removed with a waste solvent liquid and remaining microparticles above the specified size threshold are isolated as a concentrated slurry; and d) continuously recirculating the concentrated slurry from the continuous liquid centrifuge to the quench vessel, whereupon entering the quench vessel, the concentrated slurry is rinsed with water or mixed with surface treatment phase; e) directly feeding the liquid dispersion to a reactor vessel connected to a TWHFTFF, wherein a portion of the liquid dispersion containing solvent and microparticles below a specified-size threshold are removed as a permeate; and f) transferring the retentate to a holding tank.

In an alternative embodiment, the liquid dispersion from step (e) is directly fed to a reactor vessel connected to a hollow flow fiber (HFF).

Referring to FIG. 1I, in some embodiments, a process for producing microparticles 1010 is provided that includes feeding the dispersed phase and continuous phase into a mixer to form an emulsion 1020, and transferring the emulsion into quench vessel 1030 wherein it is further mixed with an extraction phase 1040. In some embodiments, the quench vessel is a batch reactor, filter reactor, or a stir tank. Upon mixing, the solvent from the dispersed phase is extracted into the extraction phase and microparticles are formed in a liquid dispersion.

Following mixing of the emulsion with the extraction phase in the quench vessel to form a liquid dispersion containing microparticles 1040, the process further includes transferring the liquid dispersion from the quench vessel to either a continuous liquid centrifuge or a parallel bank of centrifuges to form a concentrated slurry 1050. In certain embodiments, the quench vessel and centrifuge are arranged in tandem, that is, in direct fluid communication with each other. In some embodiments, the quench vessel and centrifuge are directly connected through a conduit that allows for the liquid dispersion to exit the quench vessel and enter the centrifuge. The types of centrifuges appropriate for this application are known to those having skill in the art. The rotational speed of the centrifuge will typically determine the size range for the microparticles that are isolated therein. In typical embodiments, the rotational speed is from about 2000 rpm to about 3000 rpm.

In some embodiments, the centrifuge is a filtration centrifuge or a sedimentation centrifuge. In some embodiments, the liquid dispersion from the outlet of the quench vessel is diverted to a first centrifuge in a parallel bank of two or more centrifuges. After a set centrifugation time, the liquid dispersion from the outlet of the quench vessel is diverted into one or more additional centrifuges instead of the first centrifuge. This may be required, for example, upon saturation of the centrifuge barrel with concentrated slurry in a first centrifuge in order to maintain sufficient isolation of the microparticles as a concentrated slurry. In some embodiments, the conduit from the quench vessel to the first centrifuge contains a valve, for example a T valve that allows for diversion of the liquid dispersion from the quench vessel to a second centrifuge instead of the first centrifuge. In some embodiments, the liquid dispersion is instead divided among two or more parallel centrifuges that are running concurrently. This may be accomplished by splitting the conduit from the quench vessel into several conduit lines among two or more parallel centrifuges. In some embodiments, the concentrated slurry present within the first centrifuge is optionally rinsed with a wash phase while the liquid dispersion is being diverted to one or more additional centrifuges within the parallel bank. The wash phase may be of the same composition as the extraction phase used prior or may be a different solvent composition such as those described for the dispersed phase or the continuous phase as deemed appropriate for the particular application. In some embodiments, the wash phase is water. FIG. 1B and FIG. 1C provide non-limiting examples of centrifuges. An exemplary centrifuge is the Viafuge® Pilot available from Pneumatic

Scale Angelus.

Referring again to FIG. 1I, upon entry of the liquid dispersion containing microparticles into the centrifuge, the process includes removing a portion of the dispersion as supernatant. The supernatant can be sent to waste or, in certain embodiments, recycled for further use. The concentrated slurry remaining within the centrifuge is subsequently recirculated back to quench vessel and the concentrated slurry is rinsed and optionally mixed with surface treatment phase 1550. In some embodiments, the microparticles are recirculated through the centrifuge and the quench vessel once, twice, or three times.

Referring again to FIG. 1I, following centrifugation, the process includes continuously transferring the concentrated slurry of microparticles to a second quench vessel and further to a thick wall hollow fiber tangential flow filter 1070. Upon entry of the microparticle containing liquid dispersion into the thick wall hollow fiber tangential flow filter, a portion of the dispersion and microparticles below the filtration size of the filter are removed as permeate. The permeate can be sent to waste, or, in certain embodiments, recycled for further use. The retentate containing microparticles above a certain size threshold and the remaining liquid dispersion exits the thick wall hollow fiber tangential flow filter and transferred to a holding tank 1080. Once received in the holding tank, the retentate can be further concentrated by recirculating the retentate back through the thick wall hollow fiber tangential flow filter 1090. In an alternative embodiment, the concentrated slurry of microparticles is transferred to hollow-fiber-filter (HFF).

Also provided herein is a system, system components, and an apparatus for producing and processing microparticles as described herein. FIG. 1J represents one non-limiting embodiment of a system 1110 for producing microparticles according to the processes described herein. In some embodiments, the system incorporates one or more of the system elements described in FIG. 1I.

Referring to FIG. 1J, in some embodiments, system 1110 includes a dispersed phase holding tank 1210 and a continuous phase holding tank 1220. The dispersed phase holding tank 1210 includes at least one outlet, and is capable of mixing one or more active agents, one or more solvents for the active agent, one or more polymers, and one or more solvents for the polymer to form a dispersed phase. Likewise, the continuous phase holding tank 1220 contains at least one outlet. The dispersed phase holding tank 1210 is in fluid communication with a mixer 1300 via conduit 1211. Likewise, the continuous phase holding tank 1220 is in fluid communication with mixer 1300 via conduit 1221. Conduit 1211 and 1221 may further include a filtering device 1212 and 1222, respectively, for sterilizing the phases before entry into mixer 1300. In some embodiments, the filtering device is any suitable filter for use to sterilize the phases, for example a PVDF capsule filter.

Mixer 1300 can be any suitable mixer for mixing the dispersed phase with the continuous phase to form either an emulsion or microparticles in a liquid dispersion. In some embodiments, mixer 1300 is an in-line high shear mixer. The mixer 1300 receives the dispersed phase and the continuous phase and mixes the two phases. In some embodiments, the mixer 1300 includes at least one outlet for transferring the formed emulsion or microparticles in liquid dispersion to a quench vessel 1400. The formed emulsion or microparticles contained in the liquid dispersion are transferred from the mixer 1300 to quench vessel 1400 via conduit 1311. Quench vessel 1400 includes inlet 1410 for receiving the formed emulsion or microparticles in the liquid dispersion, and one or more inlets distal to inlet 1410 for receiving extraction phase. Referring to FIG. 1J, extraction phase holding tank 1401 transfers extraction phase to the quench vessel inlet 1407 via conduit 1403. Conduit 1403 may further include a suitable sterilization filter 1405, for example as previously described, for filtering the extraction phase prior to entering the quench vessel 1400.

The quench vessel 1400 includes outlet 1409 for transferring the liquid dispersion including microparticles from the quench vessel 1400 to a centrifuge 1500. The quench vessel is in direct fluid communication with centrifuge 1500 via conduit 1413. Conduit 1413 includes a first inlet 1501 and a quench vessel outlet 1409. During processing, the liquid dispersion including microparticles is transferred from the quench vessel 1400 and enters the centrifuge 1500 via conduit 1413. The centrifuge includes a first outlet 1502 proximate to a second outlet 1505. Upon entry into the centrifuge, supernatant is removed through outlet 1502. In some embodiments, supernatant is transferred to a waste tank 1504 through outlet 1502. The centrifuge also includes a third outlet 1515 for recirculating the concentrated slurry back to quench vessel 1400 via conduit 1411. Conduit 1411 includes a first inlet 1412 connected to quench vessel 1400. In some embodiments, the concentrated slurry is recirculated from centrifuge 1500 to quench vessel 1400 via conduit 1411 and the concentrated slurry is rinsed with water. In some embodiments, quench vessel 1400 contains water prior to the recirculation of the concentrated slurry. In some embodiments, the concentrated slurry is rinsed with water or further extraction phrase. Extraction phase holding tank 1401 transfers additional extraction phase via conduit 1403. A peristaltic pump 1422 is used to allow return of the suspension toward the quench vessel via conduit 1411.

Referring again to FIG. 1J, the liquid dispersion is again transferred to centrifuge 1500 and concentrated. In some embodiments, the concentrated slurry is again recirculated to quench vessel 1400 via conduit 1411 and treated with surface treatment phase. Surface treatment is added via surface treatment holding tank 1602. Surface treatment holding tank 1602 is connected to quench vessel 1400 via conduit 1606. Conduit 1606 contains outlet 1604 connected to surface treatment holding tank 1602 and inlet 1608 connected to quench vessel 1400. Conduit 1606 also optionally contains sterilization filter 1605. The liquid dispersion of surface treated microparticles is transferred from quench vessel 1400 to centrifuge 1500 via conduit 1413 to form a concentrated slurry. The concentrated slurry is then transferred to a second quench vessel 1704 via conduit 1701. Referring to FIG. 1J, the second quench vessel 1704 includes outlet 1705 for transferring the liquid dispersion including microparticles from the second quench vessel 1704 to thick wall hollow fiber tangential flow filter 4330. The second quench vessel 1704 is in direct fluid communication with thick wall hollow fiber tangential flow filter 4330 via conduit 1716. Conduit 1716 includes a first inlet 1715 connected to second quench vessel 1704. Conduit 1716 includes outlet 1719 connected to the thick wall hollow fiber tangential flow filter 4330 at thick wall hollow fiber tangential flow filter inlet 1720. During processing, the liquid dispersion including the microparticles is transferred from the second quench vessel 1704 and enters the thick wall hollow fiber tangential flow filter 4300 via conduit 1716. The thick wall hollow fiber tangential flow filter includes a first outlet 1708 proximate to a second outlet 1731. Upon entry into the thick wall hollow fiber tangential flow filter 4330, permeate and microparticles below a certain threshold are removed as permeate through outlet 1708. In some embodiments, the permeate is transferred to a waste tank 1710 via conduit 1709. Alternatively, the permeate can be recycled.

As described above, the thick wall hollow fiber tangential flow filter 4330 is preferably a thick wall hollow fiber tangential flow filter with a filter pore size between about 1 μm and 100 μm, and more preferably from about 1 μm to about 10 μm. In certain embodiments, the thick wall hollow fiber tangential flow filter includes a filter with a pore size of about 4 μm to 8 μm.

System 1110 further includes a holding tank 1800 connected to the thick wall hollow fiber tangential flow filter via conduit 1711. Retentate exits the thick wall hollow fiber tangential flow filter 4330 at second outlet 1731 and is transferred to holding tank 1800 via conduit 1711 through holding tank inlet 1732. Holding tank 1800 includes outlet 1734 and, optionally one or more additional inlets. As illustrated in FIG. 1J, holding tank 1800 includes additional inlet 1831 for receiving a wash phase, surface treatment phase or additional components for any further formulation steps. In some embodiments, a wash phase or surface treatment phase is added to holding tank 1800 from solvent extraction phase holding tank 1803 via conduit 1801. Conduit 1801 may further comprise a filter 1802 for sterilizing the solvent extraction phase prior to entry into holding tank 1800. Holding tank 1800 can include a mixing device for mixing the liquid dispersion including the microparticles held in the tank.

Holding tank 1800 is in further fluid communication with quench vessel 1704 via conduit 1726. Conduit 1726 connects holding tank outlet 1734 with inlet 1706 of quench vessel 1704. Upon entry of the liquid dispersion including microparticles into holding tank 1800, the direct fluid connection with quench vessel 1704 via conduit 1726 allows the liquid dispersion to be recirculated through the thick wall hollow fiber tangential flow filter to quench vessel 1704. In some embodiments, quench vessel 1704 optionally includes a micron bottom filter 1746 and the liquid dispersion is sieved through the filter to remove particles above a certain size threshold. In some embodiments, filter 1746 is a 50 μm filter. A peristaltic pump 1736 is used to allow return of the suspension toward the quench vessel via conduit 1726.

FIG. 1K represents an additional non-limiting embodiment of a system 1120 for producing microparticles according to the processes described herein. In some embodiments, the system incorporates one or more of the system elements described in FIG. 1I.

Referring to FIG. 1K, in some embodiments, system 1120 includes a dispersed phase holding tank 2210 and a continuous phase holding tank 2220. The dispersed phase holding tank 2210 includes at least one outlet, and is capable of mixing one or more active agents, one or more solvents for the active agent, one or more polymers, and one or more solvents for the polymer to form a dispersed phase. Likewise, the continuous phase holding tank 2220 contains at least one outlet. The dispersed phase holding tank 2210 is in fluid communication with a mixer 2300 via conduit 2211. Likewise, the continuous phase holding tank 2220 is in fluid communication with mixer 2300 via conduit 2221. Conduit 2211 and 2221 may further include a filtering device 2212 and 2222, respectively, for sterilizing the phases before entry into mixer 2300. In some embodiments, the filtering device is any suitable filter for use to sterilize the phases, for example a PVDF capsule filter.

Mixer 2300 can be any suitable mixer for mixing the dispersed phase with the continuous phase to form either an emulsion or microparticles in a liquid dispersion. In some embodiments, mixer 2300 is an in-line high shear mixer. The mixer 2300 receives the dispersed phase and the continuous phase and mixes the two phases. In some embodiments, the mixer 2300 includes at least one outlet for transferring the formed emulsion or microparticles in liquid dispersion to a quench vessel 2400. The formed emulsion or microparticles contained in the liquid dispersion are transferred from the mixer 2300 to quench vessel 2400 via conduit 2311. Quench vessel 2400 includes inlet 2410 for receiving the formed emulsion or microparticles in the liquid dispersion, and one or more inlets distal to inlet 2410 for receiving extraction phase. Referring to FIG. 1K, extraction phase holding tank 2401 transfers extraction phase to the quench vessel inlet 2407 via conduit 2403. Conduit 2403 may further include a suitable sterilization filter 2405, for example as previously described, for filtering the extraction phase prior to entering the quench vessel 2400.

The quench vessel 2400 includes outlet 2409 for transferring the liquid dispersion including microparticles from the quench vessel 2400 to a centrifuge 2500. The quench vessel is in direct fluid communication with centrifuge 2500 via conduit 2410. Conduit 2410 includes a first inlet 2501 and a quench vessel outlet 2409. During processing, the liquid dispersion including microparticles is transferred from the quench vessel 2400 and enters the centrifuge 2500 via conduit 2410. The centrifuge includes a first outlet 2502 proximate to a second outlet 2505. Upon entry into the centrifuge, supernatant is removed through outlet 2502. In some embodiments, supernatant is transferred to a waste tank 2504 through outlet 2502. The centrifuge also includes a third outlet 2515 for recirculating the concentrated slurry back to quench vessel 2400 via conduit 2411. Conduit 2411 includes a first inlet 2412 connected to quench vessel 2400. In some embodiments, the concentrated slurry is recirculated from centrifuge 2500 to quench vessel 2400 via conduit 2411 and the concentrated slurry is rinsed with water. In some embodiments, quench vessel 2400 contains water prior to the recirculation of the concentrated slurry. In some embodiments, the concentrated slurry is rinsed with water. Water is added via holding tank 2401. A peristaltic pump 2422 is used to allow return of the suspension toward the quench vessel via conduit 2411.

Referring again to FIG. 1K, the liquid dispersion is recirculated to centrifuge 2500 and transferred to quench vessel 2704. The second quench vessel 2704 includes inlet 2607 that is connected to conduit 2606. Conduit 2606 is connected to surface treatment phase holding tank 2602. In some embodiments, the microparticles in quench vessel 2704 are surface treated and then directly transferred to thick wall hollow fiber tangential flow filter 2700. The second quench vessel 2704 is in direct fluid communication with thick wall hollow fiber tangential flow filter 2700 via conduit 2706. Conduit 2706 includes a first inlet 2715 connected to second quench vessel 2704. Conduit 2706 includes outlet 2719 connected to the thick wall hollow fiber tangential flow filter 2700 at thick wall hollow fiber tangential flow filter inlet 2720. During processing, the liquid dispersion including the microparticles is transferred from the second quench vessel 2704 and enters the thick wall hollow fiber tangential flow filter 2700 via conduit 2706. The thick wall hollow fiber tangential flow filter includes a first outlet 2708 proximate to a second outlet 2731. Upon entry into the thick wall hollow fiber tangential flow filter 2700, permeate and microparticles below a certain threshold are removed as permeate through outlet 2708. In some embodiments, the permeate is transferred to a waste tank 2710 via conduit 2709. Alternatively, the permeate can be recycled.

System 1120 further includes a holding tank 2800 connected to the thick wall hollow fiber tangential flow filter via conduit 2711. Retentate exits the thick wall hollow fiber tangential flow filter 2700 at second outlet 2731 and is transferred to holding tank 2800 via conduit 2711 through holding tank inlet 2732. Holding tank 2800 includes outlet 2734 and, optionally one or more additional inlets. As illustrated in FIG. 1K, holding tank 2800 includes additional inlet 2831 for receiving a wash phase, surface treatment phase or additional components for any further formulation steps. In some embodiments, a wash phase or surface treatment phase is added to holding tank 2800 from solvent extraction phase holding tank 2803 via conduit 2801. Conduit 2801 may further comprise a filter 2802 for sterilizing the solvent extraction phase prior to entry into holding tank 2800. Holding tank 2800 can include a mixing device for mixing the liquid dispersion including the microparticles held in the tank.

Holding tank 2800 is in further fluid communication with second quench vessel 2704 via conduit 2726. Conduit 2726 connects holding tank outlet 2734 with second inlet 2716 of second quench vessel 2704. Upon entry of the liquid dispersion including microparticles into holding tank 2800, the direct fluid connection with second quench vessel 2704 via conduit 2726 allows the liquid dispersion to be recirculated through the thick wall hollow fiber tangential flow filter to the quench vessel. In some embodiments, quench vessel 2704 optionally includes a micron bottom filter 2746 and the liquid dispersion is sieved through the filter to remove particles above a certain size threshold. In some embodiments, filter 2746 is a 50 μm filter. A peristaltic pump 2736 is used to allow return of the suspension toward the quench vessel via conduit 2726.

FIG. 1L represents an additional non-limiting embodiment of a system 1130 for producing microparticles according to the processes described herein. In some embodiments, the system incorporates one or more of the system elements described in FIG. 1I.

Referring to FIG. 1L, in some embodiments, system 1130 includes a dispersed phase holding tank 3210 and a continuous phase holding tank 3220. The dispersed phase holding tank 3210 includes at least one outlet, and is capable of mixing one or more active agents, one or more solvents for the active agent, one or more polymers, and one or more solvents for the polymer to form a dispersed phase. Likewise, the continuous phase holding tank 3220 contains at least one outlet. The dispersed phase holding tank 3210 is in fluid communication with a mixer 3300 via conduit 3211. Likewise, the continuous phase holding tank 3220 is in fluid communication with mixer 3300 via conduit 3221. Conduit 3211 and 3221 may further include a filtering device 3212 and 3222, respectively, for sterilizing the phases before entry into mixer 3300. In some embodiments, the filtering device is any suitable filter for use to sterilize the phases, for example a PVDF capsule filter.

Mixer 3300 can be any suitable mixer for mixing the dispersed phase with the continuous phase to form either an emulsion or microparticles in a liquid dispersion. In some embodiments, mixer 3300 is an in-line high shear mixer. The mixer 3300 receives the dispersed phase and the continuous phase and mixes the two phases. In some embodiments, the mixer 3300 includes at least one outlet for transferring the formed emulsion or microparticles in liquid dispersion to a quench vessel 3400. The formed emulsion or microparticles contained in the liquid dispersion are transferred from the mixer 3300 to quench vessel 3400 via conduit 3311. Quench vessel 3400 includes inlet 3410 for receiving the formed emulsion or microparticles in the liquid dispersion, and one or more inlets distal to inlet 3410 for receiving extraction phase. Referring to FIG. 1L, extraction phase holding tank 3401 transfers extraction phase to the quench vessel inlet 3407 via conduit 3403. Conduit 3403 may further include a suitable sterilization filter 3405, for example as previously described, for filtering the extraction phase prior to entering the quench vessel 3400.

The quench vessel 3400 includes outlet 3409 for transferring the liquid dispersion including microparticles from the quench vessel 3400 to a centrifuge 3500. The quench vessel is in direct fluid communication with centrifuge 3500 via conduit 3410. Conduit 3410 includes a first inlet 3501 and a quench vessel outlet 3409. During processing, the liquid dispersion including microparticles is transferred from the quench vessel 3400 and enters the centrifuge 3500 via conduit 3410. The centrifuge includes a first outlet 3502 proximate to a second outlet 3505. Upon entry into the centrifuge, supernatant is removed through outlet 3502. In some embodiments, supernatant is transferred to a waste tank 3504 through outlet 3502. The centrifuge also includes a third outlet 3515 for recirculating the concentrated slurry back to quench vessel 3400 via conduit 3411. Conduit 3411 includes a first inlet 3412 connected to quench vessel 3400. In some embodiments, the concentrated slurry is recirculated from centrifuge 3500 to quench vessel 3400 via conduit 3411 and the concentrated slurry is rinsed with water. In some embodiments, quench vessel 3400 contains water prior to the recirculation of the concentrated slurry. In some embodiments, the concentrated slurry is rinsed with water. Water is added via holding tank 3401. A peristaltic pump 3422 is used to allow return of the suspension toward the quench vessel via conduit 3411.

Referring again to FIG. 1L, the liquid dispersion is again transferred to centrifuge 3500 and concentrated. In some embodiments, the concentrated slurry is again recirculated to quench vessel 3400 via conduit 3411 and treated with surface treatment phase. Surface treatment is added via surface treatment holding tank 3602. Surface treatment holding tank 3602 is connected to quench vessel 3400 via conduit 3606. Conduit 3606 contains outlet 3604 connected to surface treatment holding tank 3602 and inlet 3608 connected to quench vessel 3400. Conduit 3606 also optionally contains sterilization filter 3605. The liquid dispersion of surface treated microparticles is transferred from quench vessel 3400 to centrifuge 3500 via conduit 3410 to form a concentrated slurry. The concentrated slurry is then transferred to a second quench vessel 3704 via conduit 3701.

The second quench vessel 3704 is in direct fluid communication with a second centrifuge 3700 via conduit 3706. Conduit 3706 includes a first inlet 3715 connected to second quench vessel 3704. Conduit 3706 includes outlet 3719 connected to the second centrifuge 3700 at centrifuge inlet 3720. During processing, the liquid dispersion including the microparticles is transferred from the second quench vessel 3704 and enters the second centrifuge 3700 via conduit 3706. The second centrifuge includes a first outlet 3708 proximate to a second outlet 3731. Upon entry into the second centrifuge 3700, permeate and microparticles below a certain threshold are removed as permeate through outlet 3708. In some embodiments, the permeate is transferred to a waste tank 3710 via conduit 3709. Alternatively, the permeate can be recycled.

System 1130 further includes a holding tank 3800 connected to the second centrifuge via conduit 3711. Retentate exits the second centrifuge 3700 at second outlet 3731 and is transferred to holding tank 3800 via conduit 3711 through holding tank inlet 3732. Holding tank 3800 includes outlet 3734 and, optionally one or more additional inlets. As illustrated in FIG. 1L, holding tank 3800 includes additional inlet 3831 for receiving a wash phase, surface treatment phase or additional components for any further formulation steps. In some embodiments, a wash phase or surface treatment phase is added to holding tank 3800 from solvent extraction phase holding tank 3803 via conduit 3801. Conduit 3801 may further comprise a filter 3802 for sterilizing the solvent extraction phase prior to entry into holding tank 3800. Holding tank 3800 can include a mixing device for mixing the liquid dispersion including the microparticles held in the tank.

Holding tank 3800 is in further fluid communication with quench vessel 3704 via conduit 3726. Conduit 3726 connects holding tank outlet 3734 with second inlet 3716 of quench vessel 3704. Upon entry of the liquid dispersion including microparticles into holding tank 3800, the direct fluid connection with quench vessel 3704 via conduit 3726 allows the liquid dispersion to be recirculated through the thick wall hollow fiber tangential flow filter to the quench vessel. In some embodiments, quench vessel 3704 optionally includes a micron bottom filter 3746 and the liquid dispersion is sieved through the filter to remove particles above a certain size threshold. In some embodiments, filter 3746 is a 50 μm filter. A peristaltic pump 3736 is used to allow return of the suspension toward the thick wall hollow fiber tangential flow filter via conduit 3726.

Thick Wall Hollow Fiber Tangential Flow Filtration (TWHFTFF)

Thick wall hollow fiber tangential flow filtration (TWHFTFF) is a filtration technique in which the starting solution passes tangentially along the surface of the filter. A pressure difference across the filter drives components that are smaller than the pores through the filter. Components larger than the filter pores are withdrawn as a permeate, which can be discarded or further purified and recycled for later use. TWHFTFFs provide filtration processes wherein the feed stream containing the microparticle containing liquid dispersion passes parallel to the filter membrane face, and the permeate passes through the membrane while the retentate passes along the membrane. Unlike traditional tangential flow filtration processes used in microparticle formation such as standard hollow fiber filtration, the use of a TWHFTFF provides for macrofiltration, that is, filtration of a particular dispersion of greater than 1 μm and can be used for solvent removal in combination with small microparticle removal, resulting in a dispersion concentrate that is free of microparticle below a certain size threshold. Because of the larger pore size and increased wall thickness, a TWHFTFF is significantly less prone to fouling like traditional tangential flow filters that incorporate thin-walled hollow fiber filters with pore sizes of, for example, less than 1 μm, for example 0.05 μm to 0.5 μm. The larger pore size and reduced fouling aspect provides for a higher throughput of the microparticle dispersion, which reduces processing time and residence time of the formed microparticle in solvent containing medium. Furthermore, by using a thicker wall, a larger number of undesirable particulates, such as microparticles of insufficient size or formation, can be removed using a TWHFTFF without the need for additional passages through the filter.

The TWHFTFF for use herein includes parallel hollow fibers residing between an inlet chamber and an outlet chamber. The thick wall hollow fibers receive the flow through the inlet chamber and advance through a hollow fiber interior of the thick wall hollow fibers, which act to filter the liquid dispersion, producing a permeate. The filtered retentate can subsequently be transferred to the holding tank.

In some embodiments, the pore size of the TWHFTFF is between about 1 μm and 100 μm. In some embodiments, the pore size of the TWHFTFF is at least about 1 μm and 80 μm. In some embodiments, the pore size of the TWHFTFF is between about 1 μm and 25 μm. In some embodiments, the pore size of the TWHFTFF is between about 5 μm and 10 μm. In some embodiments, the pore size of the TWHFTFF is between about 2 μm and 5 μm. In some embodiments, the pore size of the TWHFTFF is between about 6 μm and 8 μm. In some embodiments, the pore size of the TWHFTFF is greater than about 5 μm but less than about 10 μm. By incorporating a larger pore size, the resultant concentration of microparticles is more uniform, allowing for a reduction in the number of additional processing steps necessary to derive at a microparticle product of desired size.

The wall thickness of the TWHFTFF provides the depth aspect of the filter, and allows for significantly more filtering capability than a standard thin-walled hollow fiber filter traditionally used in microparticle processing. In some embodiments, the TWHFTFF includes tortious paths for straining particles of certain sizes not capable of passing through to the permeate, but too small to be desirable. Thus, the tortious paths provide settling zones which still allow smaller particles to pass through to the permeate. In some embodiments, the tortious paths can be of varying width and length. In some embodiments, the wall thickness of the TWHFTFF is between about 0.15 cm and about 0.40 cm. In some embodiments, the wall thickness is between about 0.265 cm and 0.33 cm. In some embodiments, the inside diameter or lumen of the hollow fiber is between about 1.0 mm and about 7.0 mm. In some embodiments, the hollow fiber filter has an inside diameter or lumen of about 3.15 mm.

The thick wall hollow fiber can be made from any suitable material known in the art. In some embodiments, the material is a polyethylene, for example a sintered polyethylene which has a molecular structure of repeating —CH2-CH2 units and may be coated with PVDF.

An exemplary TWHFTFF is described in WO 2017/180573, and available through Spectrum Labs.

In alternative embodiments, a different type of filter may be utilized instead of a thick wall hollow fiber tangential flow filter throughout the processes described herein. For example, in certain alternative embodiments, a tangential flow filter (TFF) may be used instead of a thick wall hollow fiber tangential flow filter. In certain alternative embodiments, the tangential flow filter is a tangential flow depth filter (TFDF). In certain alternative embodiments, the tangential flow filter is a hollow fiber filter. In certain alternative embodiments, the tangential flow filter is a single-use tangential flow filter. In some alternative embodiments, the TFF is arranged in a screen channel configuration. In some alternative embodiments, the TFF is arranged in a suspended screen channel configuration. In some alternative embodiments, the TFF is arranged in an open channel configuration.

Plug Flow Reactor in Combination with a TWHFTFF

The use of a plug flow reactor in tandem with a TWHFTFF significantly reduces processing time of the microparticle, while reducing drug loading elution from the microparticle due to the combination's increased capacity for solvent extraction.

By combining a plug flow reactor, which allows for increased solvent removal prior to exiting the plug flow reactor, in tandem with a high throughput TWHFTFF for solvent removal, microparticle filtering and concentration, processing time of the formed microparticle can be greatly reduces, and drug-load loss drastically decreased.

In an alternative aspect of the present invention, provided herein is a process of producing drug-loaded microparticles in a continuous process which includes: a) continuously forming an emulsion comprising a dispersed phase and a continuous phase in a mixer, wherein the dispersed phase comprises a drug, a polymer, and at least one solvent; b) directly feeding the emulsion into a plug flow reactor, wherein upon entering the plug flow reactor, the emulsion is mixed with a solvent extraction phase to form a liquid dispersion, wherein during residence in the plug flow reactor, a portion of the solvent is extracted into the extraction phase and the microparticles are hardened; c) directly feeding the liquid dispersion to a TWHFTFF, wherein the TWHFTFF is directly in-tandem with the plug flow reactor, and wherein a portion of the liquid dispersion containing solvent and microparticles below a specified-size threshold are removed as a permeate; and d) transferring the retentate to a holding tank. In some embodiments, additional extraction phase is introduced into the plug flow reactor at one or more locations as the liquid dispersion traverses through the reactor so that a serial extraction of solvent occurs.

In an alternative embodiment, the liquid dispersion of step (c) is directly fed into a hollow-fiber-filter (HFF).

Referring to FIG. 2A, a continuous process 4010 for producing a drug-loaded microparticle generally includes combining a dispersed phase and a continuous phase in a mixer to form an emulsion 4020. The dispersed phase generally includes an active agent, a polymer, and at least one solvent. The dispersed phase and continuous phase can be derived in separate holding vessels and then combined to form an emulsion using any suitable mixing device, for example a continuous stirred-tank reactor, batch mixer, static mixer, or high shear in-line mixer. Suitable mixers for mixing the dispersed phase and continuous phase are known in the art. In some embodiments, the dispersed phase and continuous phase are derived in separate holding vessels and pumped into a high-shear in line mixer. Prior to entering the mixer, the continuous phase and dispersed phase can be passed through a sterilized filter, for example through the use of a PVDF capsule filter.

The ratio of the dispersed phase to the continuous phase, which can affect solidification rate, active agent load, the efficiency of solvent removal from the dispersed phase, and porosity of the final product, is advantageously and easily controlled by controlling the flow rate of the dispersed and continuous phases into the mixer. The actual ratios of continuous phase to dispersed phase will depend upon the desired product, the polymer, the drug, the solvents, etc., and can be determined empirically by those of ordinary skill in the art. For example, the ratio of continuous phase to dispersed phase will typically range from about 5:1 to about 200:1. In some embodiments, the ratio of continuous phase to dispersed phase is about 5:1, 10:1, 20:1, 30:1, 40:1, 50:1, 60:1, 70:1, 80:1, 90:1, 100:1, 120:1, 140:1, 160:1, 180:1, or 200:1. This translates into flow rates for the dispersed phase of from about 400 mL/min. to about 10 mL/min., with a continuous phase flow rate fixed at 2000 mL/min. In another embodiment, the combined flow rate of the continuous phase and the dispersed phase is about 2000 mL/min to about 3000 mL/min. If the continuous phase flow rate is increased, the dispersed phase flow rate will change accordingly.

Referring again to FIG. 2A, in some embodiments, process 4010 includes continuously feeding the dispersed phase and continuous phase into the mixer to form an emulsion 4020, which is continuously transferred into a plug flow reactor 4030. Plug flow reactors, also referred to as continuous tubular reactors or piston flow reactors, are known in the art and provide for the interactions of materials in continuous, flowing systems of cylindrical geometry. The use of a plug flow reactor allows for the same residence time for all fluid elements in the tube. Comparatively, the use of holding vessels or stir tanks for mixing or solvent removal leads to different residence times and uneven mixing. Complete radial mixing as present in plug flow eliminates mass gradients of reactants and allows instant contact between reactants, often leading to faster reaction times and more controlled conditions. Additionally, complete radial mixing allows for uniform dispersion and conveyance of solids along the tube of the reactor, providing more even microparticle size formation.

In some embodiments, the plug flow reactor contains one or more apparatuses within the cylinder, for example a mixer that provides for additional mixing. For example, StaMixCo has developed a static mixer system that allows for plug flow by inducing radial mixing with a series of static grids along the tube. In another embodiment, the plug flow reactor is one in a series of plug flow reactors in direct fluid communication with each other via additional in line static mixers.

In some embodiments, the mixer may be an in-line mixer. The high-shear in-line mixer may be an impeller type apparatus, a flow restriction device that forces the continuous and dispersed phases through progressively smaller channels causing highly turbulent flow, a high frequency sonication tip or similar apparatus that will be apparent to those of ordinary skill in the art in view of this disclosure. An advantage of non-static mixers is that one can control the mixing intensity independently of the flow rates of the phases into the device. By providing adequate mixing intensity, microparticles can be quickly formed prior to exposure to extraction phase solvent. Suitable emulsification intensity can be obtained by running the impeller at least about 3,000 rpm or higher, for example 3,000 to about 10,000 rpm. The magnitude of the shear forces, and hence mixing intensity, can also be increased by adjusting the gap space between the impeller and emulsor screen or stator. Commercially available apparatuses adaptable to the instant process include in-line mixers from Silverson, Ross mixers and the like.

In some embodiments, the plug flow reactor is a continuous oscillatory baffled reactor (COBR). In general, the continuous oscillatory baffled reactor consists of a tube fitted with equally spaced baffles presented transversely to an oscillatory flow. The baffles disrupt the boundary layer at the tube wall, whilst oscillation results in improved mixing through the formation of vortices. By incorporating a series of equally spaced baffles along the tube, eddies are created when liquid is pushed along the tube, allowing for sufficient radial mixing.

Referring again to FIG. 2A, process 4010 further includes continuously transferring the emulsion formed in 4020 into the plug flow reactor 4030, wherein it is further mixed with a solvent extraction phase 4040. The solvent extraction phase comprises a single solvent for extracting the solvent or solvents used to formulate the dispersed phase. In some embodiments, the solvent extraction phase may comprise two or more co-solvents for extracting the solvent or solvents used to formulate the dispersed phase. Different polymer non-solvents (i.e., extraction phase), mixtures of solvents and polymer non-solvents and/or reactants for surface modification/conjugation may be used during the extraction process to produce different extraction rates, microparticle morphology, surface modification and polymorphs of crystalline drugs and/or polymers. In one aspect, the solvent extraction phase comprises water or a polyvinyl alcohol solution. In some embodiments, the solvent extraction phase comprises primarily of substantially water. The actual ratios of extraction phase to emulsion will depend upon the desired product, the polymer, the drug, the solvents, etc., and can be determined empirically by those of ordinary skill in the art. For example, the ratio of extraction phase to emulsion phase is 2:1. This translates into a flow rate of about 4000 mL/min for the extraction phase when the flow rate of the emulsion upon entry into the plug flow reactor is about 2000 mL/min. A typical plug flow reactor as used in the present invention is 0.5 inches in diameter and can range from 0.5 meters to 30 meters in length depending on the desired residence time. In some embodiments, the plug flow reactor length is about 0.5 meters to about 30 meters, about 3 meters to about 27 meters, about 5 meters to about 25 meters, about 10 meters to about 20 meters, or about 15 meters to about 18 meters. Residence times within the plug flow reactor can range from about 10 seconds to about 30 minutes depending on the desired application. In some embodiments, the residence time is about 10 seconds, about 20 seconds, about 1 minute, about 2 minutes, about 5 minutes, about 10 minutes, about 20 minutes, about 25 minutes, or about 30 minutes. In some embodiments, only one solvent extraction phase is introduced into a plug flow reactor with a length of about 0.5 meters and a residence time of about 10 to 20 seconds up to about 2.5 minutes. In an additional embodiment, solvent extraction phase and surface treatment solution are introduced into a plug flow reactor with a length between of about 30 meters and a residence time between 25 and 35 minutes.

Referring again to FIG. 2A, as the emulsion is fed into the plug flow reactor 4030, the solvent extraction phase is introduced into the plug flow reactor and the emulsion and solvent extraction phase are continually mixed 4040. Upon mixing, the solvent extraction phase, the solvent from the dispersed phase is extracted into the solvent extraction phase and microparticles are formed in a liquid dispersion. The traversal and continuous mixing of the liquid dispersion as it traverses the plug flow reactor further assists in continuous solvent removal and microparticle hardening. By using a plug flow reactor, residence time of the microparticle in the liquid dispersion can be tightly controlled, allowing for the consistent production of microparticles.

In some embodiments, one or more further solvent extraction phases are added into the plug flow reactor distally from the initial addition. The incorporation of additional solvent extraction phases can further assist in solvent extraction, resulting in a full extraction prior to the exiting of the liquid dispersion from the plug flow reactor.

Referring again to FIG. 2A, one or more surface treatment phases are optionally added 4045 distally from the solvent extraction phase into the plug flow reactor. This surface treatment is typically added to facilitate aggregation of the formed microparticles when used in their intended application.

Following the traversal of the liquid dispersion containing the microparticles through the plug flow reactor, the liquid dispersion exits the plug flow reactor and is fed directly into a thick wall hollow fiber tangential flow filter 4050. In certain embodiments, the plug flow reactor and thick wall hollow fiber tangential flow filter are arranged in tandem, that is, in direct fluid communication with each other. In some embodiments, the plug flow reactor and thick wall hollow fiber tangential flow filter are directly connected through a conduit which allows for the liquid dispersion to exit the plug flow reactor and enter the thick wall hollow fiber tangential flow filter.

Referring again to FIG. 2A, upon entry of the microparticle containing liquid dispersion into the thick wall hollow fiber tangential flow filter, a portion of the dispersion and microparticles below the filtration size of the filter are removed as permeate. The permeate can be sent to waste, or, in certain embodiments, recycled for further use. The retentate containing microparticles above a certain size threshold and the remaining liquid dispersion exits the thick wall hollow fiber tangential flow filter and transferred to a holding tank 4060. The flow rate for permeate removal through the TWHFTFF will depend upon the desired product, the polymer, the drug, the solvents, filter pore size, etc., and can be determined empirically by those of ordinary skill in the art. For example, the flow rate for permeate removal can range from about 2000 mL/min to about 5000 mL/min. The flow rate for permeate removal is usually less than the flow rate exiting the plug flow reactor as is necessary for proper flow of the retentate into the holding tank.

Once received in the holding tank, the retentate can be further concentrated by recirculating the retentate back through the thick wall hollow fiber tangential flow filter 4070. Accordingly, the holding tank includes an outlet in fluid communication with a conduit from the plug flow reactor to the thick wall hollow fiber tangential flow filter such that the retentate can be sent from the holding tank back through the thick wall hollow fiber tangential flow filter. The recirculation can occur following the completion of the continuously produced microparticles. For example, following completion of microparticle formation, all retentate is collected in the holding tank and then recirculated back through the thick wall hollow fiber tangential flow filter for further concentration and washing. Alternatively, recirculation through the thick wall hollow fiber tangential flow filter can be performed continuously, for example, as a continuous process such that as soon as the retentate is received in the holding tank, it is recirculated back through the thick wall hollow fiber tangential flow filter as the microparticle batch processing continues.

In some embodiments, no additional solvent is added to the retentate once it reaches the holding tank. In some embodiments, the holding tank may contain a wash phase. For example, the retentate exiting the thick wall hollow fiber tangential flow filter may be transferred to a holding tank containing a pre-determined amount of a wash phase. Alternatively, a wash phase may be added to the holding tank upon entry of the retentate. Additionally, the holding tank may include a starting amount of a wash phase, and as recirculation occurs, an additional amount of wash phase is continuously added. If additional washing of the microparticles within the retentate is desired, the wash phase is typically added at the same flow rate as for permeate removal during recirculation through the thick hollow fiber tangential flow filter. If concentration of the microparticles within the retentate is instead desired, no wash phase is added upon recirculation. The wash phase may be of the same composition as the solvent extraction phase used prior or may be a different solvent composition such as those described for the dispersed phase or the continuous phase as deemed appropriate for the particular application. In some embodiments, the wash phase is water. Alternatively, the retentate may also instead be optionally treated with a surface treatment solution during recirculation either in addition to or in replacement of the additional solvent extraction phase.

In another aspect of the present invention, a surface treatment phase may be optionally added to the retentate containing microparticles when present within the holding tank.

Following completion of microparticle solvent removal and concentration, the microparticles can be further processed, for example, by washing and re-concentration or by additional formulation steps.

Also provided herein is a system, system components, and an apparatus for producing and processing microparticles as described herein. FIG. 2B represents one embodiment of a system 4100 for producing microparticles according to the processes described herein. In some embodiments, the system incorporates one or more of the system elements described in FIG. 2B, for example, in some embodiments the system comprises a plug flow reactor in tandem with a thick wall hollow fiber tangential flow filter having a pore size greater than about 1 μm.

Thus, provided herein is a system and apparatus for producing and processing microparticles comprising: a) a mixer suitable for receiving and combining a dispersed phase and a continuous phase to form an emulsion; b) a plug flow reactor in direct fluid communication with the mixer via a first conduit, the plug flow reactor including a first inlet for receiving the emulsion, a second inlet proximate to the first inlet for receiving an extraction phase solvent, wherein the plug flow reactor includes one or more mixers capable of mixing the emulsion and solvent extraction phase to produce microparticles in a liquid dispersion, and an outlet; c) a tangential-flow depth filter having an inlet, a first outlet proximate to the plug flow reactor, and a second outlet distal to the plug flow reactor, wherein the tangential-flow depth filter inlet is in direct fluid communication with the outlet of the plug flow reactor via a second conduit and is capable of receiving the liquid dispersion, wherein the first outlet of the tangential-flow depth filter is capable of removing permeate, and wherein the second conduit has a first inlet connected to the plug flow reactor and second inlet distal from the first inlet; and d) a holding tank which is capable of receiving the retentate from the tangential-flow depth filter, wherein the holding tank has a first inlet in direct fluid communication via a third conduit with the second outlet of the tangential-flow depth filter, and a first outlet, wherein the first outlet is in direct fluid communication via a fourth conduit with the second inlet of the second conduit

In another aspect of the invention, provided herein is an apparatus for producing and processing microparticles comprising: a) a mixer; b) a plug flow reactor in direct fluid communication with the mixer; c) a TWHFTFF in direct fluid communication with the plug flow reactor; d) a holding tank in direct fluid communication with the TWHFTFF; and optionally e) a recirculating loop between the holding tank and the TWHFTFF.

Referring to FIG. 2B, in some embodiments, system 4100 includes a dispersed phase holding tank 4210 and a continuous phase holding tank 4220. The dispersed phase holding tank 4210 includes at least one outlet, and is capable of mixing one or more active agents, one or more solvents for the active agent, one or more polymers, and one or more solvents for the polymer to form a dispersed phase. Likewise, the continuous phase holding tank 4220 includes at least one outlet. The dispersed phase holding tank is in fluid communication with a mixer 4300 via conduit 4211. Likewise, the continuous phase holding tank is in fluid communication with mixer 4300 via conduit 4221. Conduit 4211 and 4221 may further include a filtering device 4212 and 4222, respectively, for sterilizing the phases before entry into the mixer 4300. In some embodiments, filtering devices 4212 and 4222 are any suitable filter for use to sterilize the phases, for example a PVDF capsule filter.

Mixer 4300 can be any suitable mixer for mixing the dispersed phase with the continuous phase to form either an emulsion or microparticles in a liquid dispersion. In some embodiments, the mixer 4300 is an in-line high shear mixer. The mixer 4300 receives the dispersed phase and the continuous phase and mixes the two phases. In some embodiments, the mixer 4300 includes at least one outlet for transferring the formed emulsion or microparticles in liquid dispersion to plug flow reactor 4400. The formed emulsion or microparticles contained in the liquid dispersion are transferred from the mixer 4300 to the plug flow reactor 4400 via conduit 4311. Plug flow reactor 4400 includes inlet 4410 for receiving the formed emulsion, and one or more inlets distal to inlet 4410 for receiving extraction phase solvent. Referring to FIG. 2B, solvent extraction phase holding tank 4230 transfers solvent extraction phase to the plug flow reactor inlet 4420 via conduit 4231. Conduit 4231 may further include a suitable sterilization filter 4232, for example as previously described, for filtering the solvent extraction phase prior to entering the plug flow reactor 4400.

Depending on the type of plug flow reactor used, the plug flow reactor 4400 may include one or more optional mixers. An embodiment of a plug flow reactor 4400 with one or more additional mixers is illustrated in FIG. 2C. Referring to FIG. 2C, one or more additional mixers can be positioned within the plug flow reactor to further assist in mixing the emulsion or microparticles in liquid dispersion with the solvent extraction phase. For example, mixer 4421 is placed distally from inlet 4420, allowing additional mixture of the emulsion or microparticles in liquid dispersion with the solvent extraction phase. In certain embodiments, additional mixers can be placed distally from mixer 4421, for example as illustrated by mixers 4422 and 4423.

The plug flow reactor may include additional inlets for receiving solvent extraction phase. For example, as illustrated in FIG. 2D, additional inlets distal from inlet 4420 may be included in the plug flow reactor 4400. For example, additional solvent extraction phase holding tanks 4235 and 4238 can transfer additional solvent extraction phase in two different locations distally from initial solvent extraction phase inlet 4420, for example, at inlets 4440 and 4450, respectively, via conduit 4237 and 4240. By introducing additional solvent extraction phase inlets proximate to a mixer, upon addition of the solvent extraction phase, the solvent extraction phase can be thoroughly mixed with the liquid dispersion as it traverses the plug flow reactor, providing additional solvent removal to take place. The additional solvent extraction addition conduit 4237 and 4240 may optionally contain a suitable sterilization filter 4236 and 4239, respectively, for example as previously described, for filtering the solvent extraction phase prior to entering the plug flow reactor 4400.

In another embodiment, the plug flow reactor may comprise a series of plug flow reactors in direct fluid communication via a series of static mixers. For example, as illustrated in FIG. 2E, plug flow reactor 4400 may alternatively be in direct fluid communication with static mixer 4301 via outlet 4461. The microparticle dispersion formed may flow out from static mixer 4301 via conduit 4312 to a second plug flow reactor 4401 via inlet 4411. Plug flow reactor 4401 may be in direct fluid communication with static mixer 4302 via outlet 4462. The microparticle dispersion formed may flow out from static mixer 4302 via conduit 4313 to a third plug flow reactor 4402 via inlet 4412. The third plug flow filter 4402 also has outlet 4460 that is in direct fluid communication with thick hollow fiber tangential flow filter 4500.

Referring to FIG. 2B, the plug flow reactor 4400 includes outlet 4460 for transferring the liquid dispersion including microparticles from the plug flow reactor 4400 to thick wall hollow fiber tangential flow filter 4500. The plug flow reactor 4400 is in direct fluid communication with thick wall hollow fiber tangential flow filter 4500 via conduit 4461. Conduit 4461 includes a first inlet 4462 connected to plug flow reactor outlet 4460 and a second inlet 4463. Conduit 4461 includes outlet 4464 connected to the thick wall hollow fiber tangential flow filter 4500 at thick wall hollow fiber tangential flow filter inlet 4510. During processing, the liquid dispersion including the microparticles is transferred from the plug flow reactor 4400 and enters the thick wall hollow fiber tangential flow filter 4500 via conduit 4461. The thick wall hollow fiber tangential flow filter includes a first outlet 4520 proximate to a second outlet 4530. Upon entry into the thick wall hollow fiber tangential flow filter 4500, permeate and microparticles below a certain threshold are removed as permeate through outlet 4520. In some embodiments, the permeate is transferred to a waste tank 4540 via conduit 4521. Alternatively, the permeate can be recycled.

As described above, the thick wall hollow fiber tangential flow filter 4500 is preferably a thick wall hollow fiber tangential flow filter with a filter pore size between about 1 μm and 100 μm, and more preferably from about 1 μm to about 10 μm. In certain embodiments, the thick wall hollow fiber tangential flow filter includes a filter with a pore size of about 4 μm to 8 μm.

System 4100 further includes a holding tank 4600 connected to the thick wall hollow fiber tangential flow filter via conduit 4531. Retentate exits the thick wall hollow fiber tangential flow filter 4500 at second outlet 4530 and is transferred to holding tank 4600 via conduit 4531 through holding tank inlet 4610. Holding tank 4600 includes outlet 4620 and, optionally one or more additional inlets. As illustrated in FIG. 2B, holding tank 4600 includes additional inlet 4630 for receiving a wash phase, surface treatment phase or additional components for any further formulation steps. In some embodiments, a wash phase or surface treatment phase is added to holding tank 600 from solvent extraction phase holding tank 4610 via conduit 4611. Conduit 4611 may further comprise a filter 4612 for sterilizing the solvent extraction phase prior to entry into holding tank 4600. Holding tank 4600 can include a mixing device for mixing the liquid dispersion including the microparticles held in the tank.

In another embodiment, holding tank 4600 may alternatively include two additional inlets 4630 and 4634 that allow a wash phase and a surface treatment phase to be added either separately or simultaneously. As shown in FIG. 2F, solvent extraction phase is added to holding tank 4600 from solvent extraction phase holding tank 4632 via conduit 4631 and surface treatment phase is added to holding tank 4600 from surface treatment phase holding tank 4636 via conduit 4635. Conduits 4631 and 4635 may further comprise filters 4633 and 4637, respectively, for sterilizing the phases prior to entry into holding tank 4600. Alternatively, either inlets 4630 and 4634 may be used components necessary to add additional components necessary for any further formulation steps.

Holding tank 4600 is in further fluid communication with conduit 4461 via conduit 4621. Conduit 4621 connects holding tank outlet 4620 with second inlet 4463 of conduit 4461. Upon entry of the liquid dispersion including microparticles into holding tank 4600, the direct fluid connection with conduit 4463 via conduit 4621 allows the liquid dispersion to be recirculated through the thick wall hollow fiber tangential flow filter as described above. A peristaltic pump 4622 is used to allow return of the suspension toward the tick wall hollow fiber tangential flow filter via conduit 4621.

Microfluidic Droplet Generator in Combination with a Plug Flow Reactor

In an alternative embodiment, a microfluidic droplet generator is utilized to form microparticles. A microfluidic droplet generator generates significantly less solvent than commonly used processes for microparticle formation. The microfluidic droplet generator relies on microfluidics and typically pumps continuous and dispersed phases at a flow rate of approximately 10 mL/minute compared to high-shear in-line mixers that operate with continuous phase flow rates as high as 2000 mL/minute. The requirement for a minimal amount of solvents means that less solvent has to be removed later in the process, reducing the number of steps, and less solvent has to be extracted from the microparticles, reducing drug loss during the process. Furthermore, by using a microfluidic droplet generator, highly monodisperse microparticles with constant morphology, size, and drug distribution are produced, eliminating the need for filtration. Accordingly, the present invention provides consistent batches of microparticles with high levels of drug-loading and controllable drug release profiles.

In an alternative embodiment, the microfluidic droplet generator further comprises a micro-mixing channel. Flow from the typical channels in a microfluidic droplet generator are typically extremely laminar and may not alone provide sufficient mixing to produce the desired emulsion that leads to microparticle production, such as when highly viscous solvent liquids are used. In addition, while simple microfluidic droplet generators provide very uniform droplet sizes, they lack the throughput that may be desired in certain applications. In typical microfluidic droplet generators containing a micro-mixing channel, an initial larger droplet (i.e., a slug) is produced from laminar solvent mixing upon the meeting of the two solvent channels. This initial droplet is further broken down into smaller droplets by the production of turbulent flow within the micro-mixing channel. This often leads to lower monodispersity of particle size compared to microfluidic droplet generators relying purely on laminar flow mixing, but often still significantly better than the particle size distributions obtained from typical macro-mixing processes.

The turbulent flow in the micro-mixing channel may be produced using a variety of processes. In some aspects, turbulent flow is produced via passive mixing techniques to increase diffusion. Micro-mixing channels that promote passive mixing typically have a physical arrangement that allows for increased contact time or contact area between the two solvents. Representative examples of passive micro-mixers include those that use lamination (such as wedged shape inlets or 90° rotation), zigzag channels (such as elliptic-shaped barriers), 3-D serpentine structures (such as folding structures, creeping structures, stacked shin structures, multiple splitting, stretching, and recombinant flows, or unbalanced driving forces), embedded barriers (such as SMX barriers or multidirectional vortices), twisted channels (such as split-and-recombine channels), or surface chemistry (such as obstacle shapes or T-/Y-mixers). In other aspects, turbulent flow is produced using active mixing techniques. Active mixing typically involves the application of an external force to promote diffusion. Representative examples of active mixing techniques that can be used in the micro-mixing channel include acoustic or ultrasonic techniques (such as acoustically driven sidewall-trapped microbubbles or acoustic streaming induced by a surface acoustic wave), dielectrophoretic techniques (such as chaotic advection based on a Linked Twisted Map), electrokinetic time-pulsed techniques (such as chaotic electric fields or periodic electro-osmotic flow), electrohydrodynamic force techniques, thermal actuation techniques, magnetohydrodynamic flow techniques, and electrokinetic instability techniques. Microfluidic mixing processes are further described in Lee et al. “Microfluidic Mixing: a Review” International Journal of Molecular Sciences, 2011, 12(5):3263-87, incorporated herein by reference in its entirety.

In one aspect of the present invention, provided herein is a process of producing drug-loaded microparticles in a continuous process which includes a) continuously combining a dispersed phase and a continuous phase in a microfluidic droplet generator to produce droplets, wherein the dispersed phase comprises a drug, a polymer, and at least one solvent; b) directly feeding the droplets into a plug flow reactor, wherein upon entering the plug flow reactor, the droplets are mixed with a solvent extraction phase, wherein during residence in the plug flow reactor, a portion of the solvent is extracted into the extraction phase and the droplets are hardened to produce microparticles; c) exposing the microparticles to surface-treatment solution in the plug flow reactor to produce surface-treated microparticles, d) directly feeding the microparticle suspension into a dilution vessel wherein the microparticles are washed and diluted to a target filling concentration; and e) transferring the diluted microparticle suspension into an apparatus designed for a filling operation.

In an alternative embodiment, the plug flow reactor is replaced with a continuously stirred tank reactor (CSTR) or a batch vessel. In a further embodiment, the CSTR is jacketed to maintain a temperature of approximately 2-8° C.

In some embodiments, solvent extraction phase is introduced into the plug flow reactor at one or more locations as the liquid dispersion traverses through the plug flow reactor. In some embodiments, surface-treatment solution is introduced at one or more locations as the liquid dispersion traverses through the plug flow reactor.

In some embodiments, one or more microfluidic droplet generators are utilized to simultaneously produce droplets that are directly fed into the plug flow reactor. In an alternative embodiment, the droplets are directly fed into a holding vessel which is connected via a conduit to the plug flow reactor.

By using a microfluidic droplet generator, highly monodisperse droplets are consistently formed, eliminating the need for a filtering step and resulting in batches of microparticles with the same shape and size.

By using a plug flow type reactor, initial residence time of the microparticles with solvent extraction phase can be tightly controlled. Desirable microparticle drug elution characteristics can be derived and maintained by the microparticle formation process provided by the microfluidic droplet generator and in some embodiments, the subsequent further dilution of solvent through the exposure of the microparticles to further extraction solvent phase in the plug flow removal.

In one aspect of the present invention, provided herein is a system and apparatus for producing and processing microparticles comprising: a) one or more microfluidic droplet generators suitable for receiving and combining a dispersed phase and a continuous phase to form a droplet; b) a plug flow reactor in direct fluid communication with the fluidic droplet generator via a first conduit, the plug flow reactor including (i) a first inlet for receiving the droplets, (ii) a second inlet proximate to the first inlet for receiving an extraction phase solvent, wherein the plug flow reactor includes one or more mixers capable of mixing the droplets and solvent extraction phase to produce microparticles in a liquid dispersion, (iii) a third inlet proximate to the second inlet for receiving surface-treatment solution, (iv) a fourth inlet proximate to the third inlet for receiving water for quenching and washing the surface treatment process, and (v) an outlet; and c) a dilution vessel which is capable of receiving the microparticles in a liquid dispersion from the plug flow reactor via a conduit, wherein the dilution vessel has an inlet for receiving dilution phase and an outlet to transfer the diluted microparticles to an apparatus designed for a filling operation.

In one aspect of the present invention, provided herein is an apparatus for producing and processing microparticles comprising: a) one or more microfluidic droplet generators; b) a plug flow reactor; and c) a dilution vessel.

In an alternative aspect of the present invention, provided herein is an apparatus for producing and processing microparticles comprising: a) one or more microfluidic droplet generators; b) a continuously stirred tank reactor (CSTR); and c) a dilution vessel.

As shown in FIG. 3A, processes 5001 for the large-scale production of drug-loaded microparticles are provided. The continuous process 5001 for producing a drug-loaded microparticle generally includes combining a dispersed phase and a continuous phase in a microfluidic droplet generator to form droplets in a liquid suspension 5002. A microfluidic droplet generator contains at least one dispersed phase feeding channel and at least one continuous phase feeding channel and the channels intersect at the microchannel. At this point of intersection, a microdroplet is formed. Microfluidic droplet generators allow for the production of highly monodisperse droplets. The flow rate, pressure, and velocity of the dispersed phase and the continuous phase can be manipulated to create droplets of varying size. In some embodiments, one or more microfluidic droplet generators simultaneously produce droplets in a liquid suspension and the droplets in a liquid suspension converge on a conduit that is connected to a plug flow reactor.

The dispersed phase and continuous phase can be derived in separate holding vessels and then combined to form the microparticles using a microfluidic droplet generator, for example the Dolomite Telos® High Throughput Droplet System; the Focussed Flow Droplet Generator or the T-shaped Droplet Generator developed by Micronit; or, a Elveflow microfluidic droplet generator. Suitable microfluidic droplet generators for mixing the dispersed phase and continuous phase are known in the art. Prior to entering the microfluidic droplet generator, the continuous phase and dispersed phase can be passed through a sterilized filter, for example through the use of a PVDF capsule filter.

The ratio of the dispersed phase to the continuous phase, which can affect solidification rate, active agent load, the efficiency of solvent removal from the dispersed phase, and porosity of the final product, is advantageously and easily controlled by controlling the flow rate and pressure of the dispersed and continuous phases into the microfluidic droplet generator. The actual ratios of continuous phase to dispersed phase will depend upon the desired product, the polymer, the drug, the solvents, etc., and can be determined empirically by those of ordinary skill in the art. For example, the flow rate of the dispersed phase and the continuous phase typically ranges from about 1.0 mL/min to about 20.0 μL/min. In some embodiments, the flow rate of the dispersed phase is about 0.5 mL to about 2.0 mL/min, about 1.0 mL to about 1.75 mL/min, or about 1.25 mL/min to about 1.5 mL/min. In some embodiments, the continuous phase is about 4.0 mL/min to about 20 mL/min, about 6 mL/min to about 18 mL/min, about 8 mL/min to about 16 mL/min, or about 10 mL/min to about 14 mL min. In some embodiments the continuous phase is added in a ratio of about 2:1. In some embodiments, the continuous phase is added at a flow rate of about 1.0 mL/min and the dispersed phase is added at a flow rate of about 0.5 mL/min. In some embodiments, the continuous phase is added at a flow rate of about 1 mL/min and the dispersed phase is added at a flow rate of about 2 mL/min.

Referring again to FIG. 3A, in some embodiments, the dispersed phase and continuous phase are continuously fed into the microfluidic droplet generator to form droplets in a liquid suspension 5002, which is continuously transferred into a plug flow reactor 5003. Plug flow reactors, also referred to as continuous tubular reactors or piston flow reactors, are known in the art and provide for the interactions of materials in continuous, flowing systems of cylindrical geometry. The use of a plug flow reactor allows for the same residence time for all fluid elements in the tube. The residence time of the plug flow reactor is at least sufficient to harden the particles. In some embodiments, the residence time of the microparticles is approximately 10 minutes, approximately 15 minutes, approximately 30 minutes, approximately 45 minutes, or approximately 60 minutes. Complete radial mixing as present in plug flow eliminates mass gradients of reactants and allows instant contact between reactants, often leading to faster reaction times and more controlled conditions. Additionally, complete radial mixing allows for uniform dispersion and conveyance of solids along the tube of the reactor, providing more even microparticle size formation.

In some embodiments, the plug flow diameter is less than or equal to approximately 0.5 inches. In some embodiments, the plug flow diameter is less than or equal to approximately 0.25 inches. In some embodiments, the plug flow length is approximately less than 30 meters, less than 20 meters, less than 15 meters, less than 10 meters, less than 5 meters, or approximately less than 1 meter. In some embodiments, the plug flow length is approximately less than 1000 mm, less than 750 mm, approximately less than 500 mm, less than 250 mm, or less than 100 mm.

In some embodiments, the plug flow reactor contains one or more apparatuses within the cylinder, for example a mixer that provides for additional mixing. For example, StaMixCo has developed a static mixer system that allows for plug flow by inducing radial mixing with a series of static grids along the tube.

In some embodiments, the plug flow reactor is a continuous oscillatory baffled reactor (COBR). In general, the continuous oscillatory baffled reactor consists of a tube fitted with equally spaced baffles presented transversely to an oscillatory flow. The baffles disrupt the boundary layer at the tube wall, whilst oscillation results in improved mixing through the formation of vortices. By incorporating a series of equally spaced baffles along the tube, eddies are created when liquid is pushed along the tube, allowing for sufficient radial mixing.

In an alternative embodiment, a continuously stirred tank reactor or a bath reactor is used instead of a plug flow reactor to perform the solvent extraction and/or the surface treatment.

Referring again to FIG. 3A, the microparticles in a liquid suspension formed in 5002 is continuously transferred into the plug flow reactor 5003, wherein it is mixed with solvent extraction phase and surface-treatment solution 5004. In some embodiments, the microparticles are exposed to solvent extraction phase for approximately 1 to 10 minutes, 2 to 8 minutes, or 3 to 5 minutes. In some embodiments, the solvent extraction phase comprises a single solvent for extracting the solvent or solvents used to formulate the dispersed phase. In some embodiments, the solvent extraction phase may comprise two or more co-solvents for extracting the solvent or solvents used to formulate the dispersed phase. Different polymer non-solvents (i.e., extraction phase), mixtures of solvents and polymer non-solvents and/or reactants for surface modification/conjugation may be used during the extraction process to produce different extraction rates, microparticle morphology, surface modification and polymorphs of crystalline drugs and/or polymers. In one aspect, the solvent extraction phase comprises water or a polyvinyl alcohol solution. In some embodiments, the solvent extraction phase comprises primarily of substantially water.

Upon mixing, the solvent extraction phase, the solvent from the disperse phase is extracted into the solvent extraction phase and microparticles are formed in a liquid dispersion. The traversal and continuous mixing of the liquid dispersion as it traverses the plug flow reactor further assists in continuous solvent removal and microparticle hardening. By using a plug flow reactor, residence time of the microparticle in the liquid dispersion can be tightly controlled, allowing for the consistent production of microparticles.

In some embodiments, one or more further solvent extraction phases are added into the plug flow reactor distally from the initial addition. The incorporation of additional solvent extraction phases can further assist in solvent extraction, resulting in a full extraction prior to the exiting of the liquid dispersion from the plug flow reactor.

By using a plug flow reactor, residence time of the microparticle in the solvent extraction phase can be tightly controlled, allowing for the consistent production of microparticles.

As the emulsion is fed into the plug flow reactor 5003, the solvent extraction phase is introduced into the plug flow reactor 5004 and the droplets are first mixed with solvent extraction phase where upon mixing, the droplets solidify to microparticles. The resulting microparticles are then exposed to surface-treatment solution. Upon mixing, the microparticles are surface-treated.

Following the traversal of the liquid dispersion containing the microparticles through the plug flow reactor, the liquid dispersion exits the plug flow reactor and is fed directly into a quench and dilution vessel 5005.

By combining a microfluidic droplet generator in tandem with a plug flow reactor, highly monodisperse microparticles are produced with consistent morphology and API distribution, which is highly efficient and eliminates the need for a filtration step.

Referring again to FIG. 3A, upon entry of the microparticle-containing liquid dispersion into the dilution vessel, the suspension of microparticles is diluted to the target filling concentration and transferred to a holding tank 5006.

Following completion of microparticle solvent removal and concentration, the microparticles can be further processed, for example, by washing and re-concentration.

Also provided herein is a system and apparatus for producing and processing microparticles as described herein. FIG. 3B represents one embodiment of a system 5100 for producing microparticles according to the processes described herein. In some embodiments, the system incorporates one or more of the system elements described in FIG. 3B, for example, in some embodiments the system comprises a microfluidic droplet generator with a T-junction in tandem with a plug flow reactor.

Referring to FIG. 3B, in some embodiments, system 5100 includes a dispersed phase holding tank 5210 and a continuous phase holding tank 5220. The dispersed phase holding tank 5210 includes at least one outlet and is capable of mixing one or more active agents, one or more solvents for the active agent, one or more polymers, and one or more solvents for the polymer to form a dispersed phase. Likewise, the continuous phase holding tank 5220 includes at least one outlet. The dispersed phase holding tank 5210 is in fluid communication with the microfluidic droplet generator 5200 via conduit 5211. Likewise, the continuous phase holding tank 5220 is in fluid communication with the microfluidic droplet generator 5200 via conduit 5212. Conduit 5211 and 5212 may further include a filtering device (5222 and 5233, respectively) for sterilizing the phases before entry into the microfluidic droplet generator 5200. In some embodiments, the filtering device is any suitable filter for use to sterilize the phases, for example a PVDF capsule filter.

The microfluidic droplet generator 5200 can be any suitable microfluidic droplet generator for mixing the dispersed phase with the continuous phase to form droplets in a liquid dispersion.

In some embodiments, the microfluidic droplet generator 5200 has a T-junction microchannel 5230 with a dispersion phase feeding channel 5214 and a continuous phase feeding channel 5215 as shown in FIG. 3C. In this embodiment, the dispersion phase feeding port 5213 is placed such that the dispersion phase feeding port 5213 and the microchannel 5230 cross.

In some embodiments, the microfluidic droplet generator has a 4-prong junction microchannel 5240 with two dispersion phase feeding channels (5216 and 5217) and a continuous phase feeding channel 5218 as shown in FIG. 3D. In this embodiment, the dispersion phase feeding ports 5219 and 5241 are placed such that the dispersion phase feeding ports 5219 and 5241 and the microchannel 5240 cross.

In some embodiments, one or more microfluidic droplet generators, or a bank of microfluidic droplet generators, are connected to the plug flow reactor via conduit 5311 as shown in FIG. 3E. In this embodiment, continuous phase holding tank 5220 and dispersed phase holding tank 5210 are in communication with microfluidic droplet generator 5200 via conduits 5211 and 5212. A second microfluidic droplet generator 5201 is also connected to continuous phase holding tank 5260 via conduit 5261 and dispersed phase holding tank 5250 via conduit 5251. Conduit 5251 and 5261 may further include a filtering device (5252 and 5262, respectively) for sterilizing the phases before entry into the microfluidic droplet generator 5201. Droplets are produced in microfluidic droplet generator 5200 via microchannel 5230 and droplets are produced in microfluidic droplet generator 5201 via microchannel 5231. Microchannel 5230 is connected to conduit 5235 and microchannel 5231 is connected to conduit 5236. Conduits 5235 and 5236 converge on point 5237 and the convergence 5237 is connected to conduit 5311.

Referring again to FIG. 3B, the formed emulsion or microparticles contained in the liquid dispersion are transferred from the microfluidic droplet generator 5200 to the plug flow reactor 5400 via conduit 5311. Plug flow reactor 5400 includes inlet 5410 for receiving the formed droplets or microparticles in liquid dispersion, and one or more inlets distal to inlet 5410 for receiving solvent extraction phase. Referring to FIG. 3F, solvent phase extraction holding tank 5425 transfers solvent phase extraction to the plug flow reactor inlet 5420 via conduit 5426. Conduit 5426 may further include a suitable sterilization filter 5430, for example as previously described, for filtering the solvent extraction phase prior to entering the plug flow reactor 5400. The plug flow reactor also includes additional inlet 5440 downstream of inlet 5420 for receiving surface-treatment solution. Surface-treatment holding tank 5470 transfers surface-treatment solution to the plug flow reactor inlet 5420 via conduit 5441. Conduit 5441 may further include a suitable sterilization filter 5471, for example as previously described, for filtering the solvent extraction phase prior to entering the plug flow reactor 5400. In some embodiments, the plug flow reactor contains a jacketed portion wrapped around the plug flow reactor that contains an inlet and an outlet that allows for cooling liquid to circulate around the plug flow reactor. This allows for the maintenance of a temperature, for example a temperature of 2-8° C. In some embodiments, the plug flow reactor is NiTech's D15 LITE or STANDARD where either the straights or bends are jacketed to maintain a constant temperature.

Depending on the type of plug flow reactor used, the plug flow reactor 5400 may include one or more optional mixers. An embodiment of a plug flow reactor 5400 with one or more additional mixers is illustrated in FIG. 3F. Referring to FIG. 3F, one or more additional mixers can be positioned within the plug flow reactor to further assist in mixing the emulsion or microparticles in liquid dispersion with the surface treatment solution. For example, mixer 5421 is placed distally from inlet 5420, allowing additional mixture of the emulsion or microparticles in liquid dispersion with the solvent extraction phase. In certain embodiments, additional mixers can be placed distally from mixer 5421, for example as illustrated by mixers 5422, and 5423.

The plug flow reactor may include additional inlets for receiving surface-treatment solution. For example, as illustrated in FIG. 3G, additional inlets proximal from inlet 5440 may be included in the plug flow reactor 5400. For example, surface-treatment holding tank 5480 can transfer additional surface-treatment solution in one or more locations proximally from initial solvent extraction phase inlet 5440, for example, at inlet 5450, via conduit 5451. Additional locations for surface-treatment solution additions can be utilized.

In another embodiment, the plug flow reactor may comprise a series of plug flow reactors in direct fluid communication via a series of static mixers. For example, as illustrated in FIG. 3H, plug flow reactor 5401 may be in direct fluid communication with static mixer 5403 via outlet 5435. The microparticle dispersion formed may flow out from static mixer 5403 via conduit 5404 to a second plug flow reactor 5406 via inlet 5411. The second plug flow reactor 5406 may be in direct fluid communication with a second static mixer 5405 via outlet 5436. The microparticle dispersion formed may flow out from static mixer 5405 via conduit 5407 to a third plug flow reactor 5408 via inlet 5412. The third plug flow filter 5408 is in direct fluid communication with dilution vessel 5500 via conduit 5413.

In an alternative embodiment, the microparticles are directly transferred from the microfluidic droplet generator to a continuously stirred tank reactor (CSTR) or a batch vessel.

Referring to FIG. 3B, the plug flow reactor 5400 includes outlet 5460 for transferring the liquid dispersion including microparticles from the plug flow reactor 5400 to dilution vessel 3500. The plug flow reactor 5400 is in direct fluid communication with the dilution vessel 5500 via conduit 5461. Conduit 5461 includes a first inlet 5462 connected to plug flow reactor outlet 5460. During processing, the liquid dispersion including the microparticles is transferred from the plug flow reactor 5400 and enters the dilution vessel 5500 via conduit 5461.

In some embodiments, dilution vessel 5500 includes additional inlets 5530 and 5550 for receiving additional surface treatment solution and/or dilution phase. For example, as illustrated in FIG. 3I, additional surface treatment solution is added to dilution vessel 5500 from surface treatment holding tank 5520 via conduit 5511. Conduit 5511 may further comprise a filter 5512 for sterilizing the solvent extraction phase prior to entry into dilution vessel 5500. As further illustrated in FIG. 3I, additional dilution phase is added to holding tank 5500 from dilution phase holding tank 5560 via conduit 5562. Conduit 5562 may further comprise a filter 5561 for sterilizing the dilution phase prior to entry into dilution vessel 5500.

Dilution vessel 5500 can include a mixing device for mixing the liquid dispersion including the microparticles held in the tank. Dilution vessel 5500 further includes outlet 5540 for transferring the microparticle suspension that has been diluted to the appropriate filing concentration, from the dilution vessel into an apparatus designed for filling operation.

Microfluidic Droplet Generator in Combination with a Centrifuge

In another aspect of the present invention, a parallel bank of centrifuges or a continuous liquid centrifuge is used in conjugation with a microfluidic droplet generator. In this embodiment, the process of producing drug-loaded microparticles in a continuous process includes a) continuously combining a dispersed phase and a continuous phase in a microfluidic droplet generator to produce droplets, wherein the dispersed phase comprises a drug, a polymer, and at least one solvent; b) directly feeding the droplets into a plug flow reactor, wherein upon entering the plug flow reactor, the droplets are mixed with a solvent extraction phase, wherein during residence in the plug flow reactor, a portion of the solvent is extracted into the extraction phase and the droplets are hardened to produce microparticles; c) exposing the microparticles to surface-treatment solution in the plug flow reactor to produce surface-treated microparticles, d) directly feeding the liquid dispersion to a reactor vessel connected to a continuous liquid centrifuge or a parallel bank of centrifuges via an outlet from the reactor vessel, wherein a portion of the liquid dispersion containing solvent and microparticles below a specified size threshold are removed with a waste solvent liquid and remaining microparticles above the specified size threshold are isolated as a concentrated slurry; and e) transferring the concentrated slurry into an apparatus designed for a washing and filling operation.

Referring to FIG. 3J, dilution vessel 5500 is directly connected to centrifuge 5800 via conduit 5803 and microparticles are further processed via centrifugation. The liquid dispersion containing the microparticles are transferred from dilution vessel 5550 to centrifuge 5800 via conduit 5803. Conduit 5803 includes outlet 5540 that is connected to dilution vessel 5500 and outlet 5802 connected to centrifuge 5800. The centrifuge includes a first outlet 5804 proximate to a second outlet 5807. Upon entry into the centrifuge, supernatant is removed through outlet 5804. In some embodiments, supernatant is transferred to a waste tank 5806 through outlet 5804. Centrifuge 5800 is in further fluid communication with dilution vessel 5500 via conduit 5813. Upon centrifugation, the direct fluid connection with dilution vessel 5500 via conduit 5813 allows the liquid dispersion to be recirculated through the dilution vessel and the centrifuge. A peristaltic pump 5814 is used to allow return of the suspension toward the dilution vessel via conduit 5813.

The concentrated slurry is then transferred to holding tank 5811 via conduit 5808 for further processing.

In an alternative aspect of the present invention, a thick wall hollow fiber tangential flow filtration (TWHFTFF) is used in conjugation with a microfluidic droplet generator. In this embodiment, the process of producing drug-loaded microparticles in a continuous process includes a) continuously combining a dispersed phase and a continuous phase in a microfluidic droplet generator to produce droplets, wherein the dispersed phase comprises a drug, a polymer, and at least one solvent; b) directly feeding the droplets into a plug flow reactor, wherein upon entering the plug flow reactor, the droplets are mixed with a solvent extraction phase, wherein during residence in the plug flow reactor, a portion of the solvent is extracted into the extraction phase and the droplets are hardened to produce microparticles; c) exposing the microparticles to surface-treatment solution in the plug flow reactor to produce surface-treated microparticles, d) directly feeding the liquid dispersion to a reactor vessel connected to a thick wall hollow fiber tangential flow filtration (TWHFTFF) via an outlet from the reactor vessel, wherein a portion of the liquid dispersion containing solvent and microparticles below a specified size threshold are removed with a waste solvent liquid and remaining microparticles above the specified size threshold are isolated as a concentrated slurry; and e) transferring the concentrated slurry into an apparatus designed for a washing and filling operation.

In an alternative process, the liquid dispersion of step (d) is fed into a reactor vessel connected to a hollow flow fiber (HFF).

Therapeutically Active Agents to be Delivered

The microparticles prepared according to the processes disclosed herein may include an effective amount of a therapeutically active agent that can be used to treat any selected disease or disorder in a subject, typically a human, or an animal, for example a mammal. In one embodiment, the subject is a human. In one embodiment, the active agent is useful for the treatment of an ocular disease or disorder.

Non-limiting examples of ocular disorders that can be treated with microparticles made according to the disclosed process include, but are not limited to glaucoma, a disorder or abnormality related to an increase in intraocular pressure (IOP), a disorder mediated by nitric oxide synthase (NOS), a disorder requiring neuroprotection such as to regenerate/repair optic nerves, allergic conjunctivitis, anterior uveitis, cataracts, dry or wet age-related macular degeneration (AMD), geographic atrophy or diabetic retinopathy, or an inflammatory or autoimmune disorder.

Non-limiting examples of methods of administration of these microparticles to the eye include intravitreal, intrastromal, intracameral, sub-tenon, sub-retinal, retro-bulbar, peribulbar, suprachoroidal, choroidal, subchoroidal, conjunctival, subconjunctival, episcleral, posterior juxtascleral, circumcorneal, and tear duct injections, or through a mucus, mucin, or a mucosal barrier.

In an alternative embodiment, the microparticles may be delivered systemically, topically, parentally, subcutaneously, buccally, or sublingually.

In one embodiment, the microparticle can be used for the treatment of an abnormal cellular proliferation, including a tumor, cancer, an autoimmune disease, or an inflammatory disease. The active agents can be provided in the form a pharmaceutically acceptable salt. A “pharmaceutically acceptable salt” is formed when a therapeutically active compound is modified by making an inorganic or organic, non-toxic, acid or base addition salt thereof. Salts can be synthesized from a parent compound that contains a basic or acidic moiety by conventional chemical methods. Generally, such a salt can be prepared by reacting a free acid form of the compound with a stoichiometric amount of the appropriate base (such as Na, Ca, Mg, or K hydroxide, carbonate, bicarbonate, or the like), or by reacting a free base form of the compound with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like. Lists of additional suitable salts may be found, e.g., in Remington's Pharmaceutical Sciences, 17th ed., Mack Publishing Company, Easton, Pa., p. 1418 (1985).

In one embodiment, the active agent is in the form of a prodrug. Examples of prodrugs are disclosed in US Application US 2018-0036416 and PCT Applications WO 2018/175922 assigned to Graybug Vision Inc., and are specifically incorporated by reference. For example, the active agents, as described herein, may include, for example, prodrugs, which are hydrolysable to form the active beta-blockers Timolol, Metipranolol, Levobunolol, Carteolol, or Betaxolol in vivo. The compounds, as described herein, may include, for example, prodrugs, which are hydrolysable to form Brinzolamide, Dorzolamide, Acetazolamide, or Methazolamide in vivo.

In one embodiment, the microparticles of the present invention can comprise an active agent, for instance a beta-adrenergic antagonists, a prostaglandin analog, an adrenergic agonist, a carbonic anhydrase inhibitor, a parasympathomimetic agent, a dual anti-VEGF/Anti-PDGF therapeutic or a dual leucine zipper kinase (DLK) inhibitor. In another embodiment, the microparticles of the present invention can comprise an active agent for the treatment of diabetic retinopathy.

Examples of loop diuretics include furosemide, bumetanide, piretanide, ethacrynic acid, etozolin, and ozolinone.

Examples of beta-adrenergic antagonists include, but are not limited to, timolol (Timoptic®), levobunolol (Betagan®), carteolol (Ocupress®), Betaxolol (Betoptic), and metipranolol (OptiPranolol®).

Examples of prostaglandin analogs include, but are not limited to, latanoprost (Xalatan®), travoprost (Travatan®), bimatoprost (Lumigan®) and tafluprost (Zioptan™).

Examples of adrenergic agonists include, but are not limited to, brimonidine (Alphagan®), epinephrine, dipivefrin (Propine®) and apraclonidine (Lopidine®).

Examples of carbonic anhydrase inhibitors include, but are not limited to, dorzolamide (Trusopt®), brinzolamide (Azopt®), acetazolamide (Diamox®) and methazolamide (Neptazane®).

Examples of tyrosine kinase inhibitors include Tivosinib, Imatinib, Gefitinib, Erlotinib, Lapatinib, Canertinib, Semaxinib, Vatalaninib, Sorafenib, Axitinib, Pazopanib, Dasatinib, Nilotinib, Crizotinib, Ruxolitinib, Vandetanib, Vemurafenib, Bosutinib, Cabozantinib, Regorafenib, Vismodegib, and Ponatinib. In one embodiment, the tyrosine kinase inhibitor is selected from Tivosinib, Imatinib, Gefitinib, and Erlotinib. In one embodiment, the tyrosine kinase inhibitor is selected from Lapatinib, Canertinib, Semaxinib, and Vatalaninib. In one embodiment, the tyrosine kinase inhibitor is selected from Sorafenib, Axitinib, Pazopanib, and Dasatinib. In one embodiment, the tyrosine kinase inhibitor is selected from Nilotinib, Crizotinib, Ruxolitinib, Vandetanib, and Vemurafenib. In one embodiment, the tyrosine kinase inhibitor is selected from Bosutinib, Cabozantinib, Regorafenib, Vismodegib, and Ponatinib.

An example of a parasympathomimetic includes, but is not limited to, pilocarpine.

DLK inhibitors include, but are not limited to, Crizotinib, KW-2449 and Tozasertib, see structure below.

Drugs used to treat diabetic retinopathy include, but are not limited to, ranibizumab (Lucentis®).

In one embodiment, the dual anti-VEGF/Anti-PDGF therapeutic is sunitinib.

In one embodiment, the dual anti-VEGF/Anti-PDGF therapeutic is sunitinib malate (Sutent®).

In one embodiment, the active agent is a Syk inhibitor, for example, Cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′-((5-fluoropyrimidine-2,4-diyl)bis(azanediyl))diphenol), R348 (3-Ethyl-4-methylpyridine), R406 (6-((5-fluoro-2-((3,4,5-trimethoxyphenyl)amino)pyrimidin-4-yl)amino)-2,2-dimethyl-2H-pyrido[3,2-b][1,4]oxazin-3(4H)-one), piceatannol (3-Hydroxyresveratol), YM193306 (Singh et al. Discovery and Development of Spleen Tyrosine Kinase (SYK) Inhibitors, J. Med. Chem. 2012, 55, 3614-3643), 7-azaindole, piceatannol, ER-27319 (Singh et al. Discovery and Development of Spleen Tyrosine Kinase (SYK) Inhibitors, J. Med. Chem. 2012, 55, 3614-3643 incorporated in its entirety herein), Compound D (Singh et al. Discovery and Development of Spleen Tyrosine Kinase (SYK) Inhibitors, J. Med. Chem. 2012, 55, 3614-3643 incorporated in its entirety herein), PRT060318 (Singh et al. Discovery and Development of Spleen Tyrosine Kinase (SYK) Inhibitors, J. Med. Chem. 2012, 55, 3614-3643 incorporated in its entirety herein), luteolin (Singh et al. Discovery and Development of Spleen Tyrosine Kinase (SYK) Inhibitors, J. Med. Chem. 2012, 55, 3614-3643 incorporated in its entirety herein), apigenin (Singh et al. Discovery and Development of Spleen Tyrosine Kinase (SYK) Inhibitors, J. Med. Chem. 2012, 55, 3614-3643 incorporated in its entirety herein), quercetin (Singh et al. Discovery and Development of Spleen Tyrosine Kinase (SYK) Inhibitors, J. Med. Chem. 2012, 55, 3614-3643 incorporated in its entirety herein), fisetin (Singh et al. Discovery and Development of Spleen Tyrosine Kinase (SYK) Inhibitors, J. Med. Chem. 2012, 55, 3614-3643 incorporated in its entirety herein), myricetin (Singh et al. Discovery and Development of Spleen Tyrosine Kinase (SYK) Inhibitors, J. Med. Chem. 2012, 55, 3614-3643 incorporated in its entirety herein), morin (Singh et al. Discovery and Development of Spleen Tyrosine Kinase (SYK) Inhibitors, J. Med. Chem. 2012, 55, 3614-3643 incorporated in its entirety herein).

In one embodiment, the therapeutic agent is a MEK inhibitor. MEK inhibitors for use in the present invention are well known, and include, for example, trametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026/MSC 1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEAl 19 (N-(3,4-difluoro-2-(2-fluoro-4-iodophenylamino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methylsulfamoylamino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-((3-oxo-1,2-oxazinan-2yl)methyl)benzamide), or AZD8330 (2-((2-fluoro-4-iodophenyl)amino)-N-(2 hydroxyethoxy)-1,5-dimethyl-6-oxo-1,6-dihydropyridine-3-carboxamide), U0126-EtOH, PD184352 (CI-1040), GDC-0623, BI-847325, cobimetinib, PD98059, BIX 02189, BIX 02188, binimetinib, SL-327, TAK-733, PD318088, and additional MEK inhibitors as described below.

In one embodiment, the therapeutic agent is a Raf inhibitor. Raf inhibitors for use in the present invention are well known, and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265(1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), Sorafenib N-Oxide (4-[4-[[[[4-Chloro-3(trifluoroMethyl)phenyl]aMino]carbonyl]aMino]phenoxy]-N-Methyl-2pyridinecarboxaMide 1-Oxide), PLX-4720, dabrafenib (GSK2118436), GDC-0879, RAF265, AZ 628, SB590885, ZM336372, GW5074, TAK-632, CEP-32496, LY3009120, and GX818 (Encorafenib).

In certain aspects, the therapeutic agent is an anti-inflammatory agent, a chemotherapeutic agent, a radiotherapeutic, an additional therapeutic agent, or an immunosuppressive agent.

In one embodiment, a chemotherapeutic is selected from, but not limited to, imatinib mesylate (Gleevac®), dasatinib (Sprycel®), nilotinib (Tasigna®), bosutinib (Bosulif®), trastuzumab (Herceptin®), trastuzumab-DM1, pertuzumab (Perjeta™), lapatinib (Tykerb®), gefitinib (Iressa®), erlotinib (Tarceva®), cetuximab (Erbitux®), panitumumab (Vectibix®), vandetanib (Caprelsa®), vemurafenib (Zelboraf®), vorinostat (Zolinza®), romidepsin (Istodax®), bexarotene (Tagretin®), alitretinoin (Panretin®), tretinoin (Vesanoid®), carfilizomib (Kyprolis™), pralatrexate (Folotyn®), bevacizumab (Avastin®), ziv-aflibercept (Zaltrap®), sorafenib (Nexavar®), sunitinib (Sutent®), pazopanib (Votrient®), regorafenib (Stivarga®), and cabozantinib (Cometriq™).

Additional chemotherapeutic agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: vincristine (Oncovin®) or liposomal vincristine (Marqibo®), daunorubicin (daunomycin or Cerubidine®) or doxorubicin (Adriamycin®), cytarabine (cytosine arabinoside, ara-C, or Cytosar®), L-asparaginase (Elspar®) or PEG-L-asparaginase (pegaspargase or Oncaspar®), etoposide (VP-16), teniposide (Vumon®), 6-mercaptopurine (6-MP or Purinethol®), Methotrexate, cyclophosphamide (Cytoxan®), Prednisone, dexamethasone (Decadron), imatinib (Gleevec®), dasatinib (Sprycel®), nilotinib (Tasigna®), bosutinib (Bosulif®), and ponatinib (Iclusig™). Examples of additional suitable chemotherapeutic agents include but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), chlorambucil, cisplatin, cladribine, colchicin, conjugated estrogens, cyclophosphamide, cyclothosphamide, cytarabine, cytarabine, cytochalasin B, cytoxan, dacarbazine, dactinomycin, dactinomycin (formerly actinomycin), daunirubicin HCL, daunorucbicin citrate, denileukin diftitox, Dexrazoxane, Dibromomannitol, dihydroxy anthracin dione, docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

Additional therapeutic agents can include bevacizumab, sutinib, sorafenib, 2-methoxyestradiol or 2ME2, finasunate, vatalanib, vandetanib, aflibercept, volociximab, etaracizumab (MEDI-522), cilengitide, erlotinib, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacetuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, celecoxib, bazedoxifene, AZD4547, rilotumumab, oxaliplatin (Eloxatin), PD0332991 (palbociclib), ribociclib (LEE011), amebaciclib (LY2835219), HDM201, fulvestrant (Faslodex), exemestane (Aromasin), PIM447, ruxolitinib (INC424), BGJ398, necitumumab, pemetrexed (Alimta), and ramucirumab (IMC-1121B).

In one aspect of the present invention, an immunosuppressive agent is used, preferably selected from the group consisting of a calcineurin inhibitor, e.g. a cyclosporin or an ascomycin, e.g. Cyclosporin A (NEORAL®), FK506 (tacrolimus), pimecrolimus, a mTOR inhibitor, e.g. rapamycin or a derivative thereof, e.g. Sirolimus (RAPAMUNE®), Everolimus (Certican®), temsirolimus, zotarolimus, biolimus-7, biolimus-9, a rapalog, e.g.ridaforolimus, azathioprine, campath 1H, a SIP receptor modulator, e.g. fingolimod or an analogue thereof, an anti-IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, OKT4, T10B9.A-3A, 33B3.1, 15-deoxyspergualin, tresperimus, Leflunomide ARAVA®, CTLAI-Ig, anti-CD25, anti-IL2R, Basiliximab (SIMULECT®), Daclizumab (ZENAPAX®), mizorbine, methotrexate, dexamethasone, ISAtx-247, SDZ ASM 981 (pimecrolimus, Elidel®), CTLA41g (Abatacept), belatacept, LFA31g, etanercept (sold as Enbrel® by Immunex), adalimumab (Humira®), infliximab (Remicade®), an anti-LFA-1 antibody, natalizumab (Antegren®), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

Biodegradable Polymers

The microparticles can include one or more biodegradable polymers or copolymers. The polymers should be biocompatible in that they can be administered to a patient without an unacceptable adverse effect. Biodegradable polymers are well known to those in the art and are the subject of extensive literature and patents. The biodegradable polymer or combination of polymers can be selected to provide the target characteristics of the microparticles, including the appropriate mix of hydrophobic and hydrophilic qualities, half-life and degradation kinetics in vivo, compatibility with the therapeutic agent to be delivered, appropriate behavior at the site of injection, etc.

For example, it should be understood by one skilled in the art that by manufacturing a microparticle from multiple polymers with varied ratios of hydrophobic, hydrophilic, and biodegradable characteristics that the properties of the microparticle can be designed for the target use. As an illustration, a microparticle manufactured with 90 percent PLGA and 10 percent PEG is more hydrophilic than a microparticle manufactured with 95 percent PLGA and 5 percent PEG. Further, a microparticle manufactured with a higher content of a less biodegradable polymer will in general degrade more slowly. This flexibility allows microparticles of the present invention to be tailored to the desired level of solubility, rate of release of pharmaceutical agent, and rate of degradation.

Polymers useful in producing microparticles are generally known in the art, for example as described in U.S. Pat. Nos. 4,818,542, 4,767,628, 3,773,919, 3,755,558 and 5,407,609, incorporated herein by reference. Polymer concentration in the dispersed phase will be from about 5 to about 40%, and still more preferably from about 8 to about 30%. Non-limiting examples of polymers include polyesters, polyhydroxyalkanoates, polyhydroxybutyrates, polydioxanones, polyhydroxyvalerates, poly anhydrides, polyorthoesters, polyphosphazenes, polyphosphates, polyphosphoesters, polydioxanones, polyphosphoesters, polyphosphates, polyphosphonates, polyphosphates, polyhydroxyalkanoates, polycarbonates, polyalkylcarbonates, polyorthocarbonates, polyesteramides, polyamides, polyamines, polypeptides, polyurethanes, polyalkylene alkylates, polyalkylene oxalates, polyalkylene succinates, polyhydroxy fatty acids, polyacetals, polycyanoacrylates, polyketals, polyetheresters, polyethers, polyalkylene glycols, polyalkylene oxides, polyethylene glycols, polyethylene oxides, polypeptides, polysaccharides, or polyvinyl pyrrolidones. Other non-biodegradable but durable polymers include without limitation ethylene-vinyl acetate co-polymer, polytetrafluoroethylene, polypropylene, polyethylene, and the like. Likewise, other suitable non-biodegradable polymers include without limitation silicones and polyurethanes.

In particular embodiments, the polymer can be a poly(lactide), a poly(glycolide), a poly(lactide-co-glycolide), a poly(caprolactone), a poly(orthoester), a poly(phosphazene), a poly(hydroxybutyrate) or a copolymer containing a poly(hydroxybutarate), a poly(lactide-co-caprolactone), a polycarbonate, a polyesteramide, a polyanhydride, a poly(dioxanone), a poly(alkylene alkylate), a copolymer of polyethylene glycol and a polyorthoester, a biodegradable polyurethane, a poly(amino acid), a polyamide, a polyesteramide, a polyetherester, a polyacetal, a polycyanoacrylate, a poly(oxyethylene)/poly(oxypropylene) copolymer, polyacetals, polyketals, polyphosphoesters, polyhydroxyvalerates or a copolymer containing a polyhydroxyvalerate, polyalkylene oxalates, polyalkylene succinates, poly(maleic acid), and copolymers, terpolymers, combinations, or blends thereof.

Useful biocompatible polymers are those that comprise one or more residues of lactic acid, glycolic acid, lactide, glycolide, caprolactone, hydroxybutyrate, hydroxyvalerates, dioxanones, polyethylene glycol (PEG), polyethylene oxide, or a combination thereof. In a still further aspect, useful biocompatible polymers are those that comprise one or more residues of lactide, glycolide, caprolactone, or a combination thereof. Biodegradable polymers may also comprise one or more blocks of hydrophilic or water soluble polymers, including, but not limited to, polyethylene glycol, (PEG), or polyvinyl pyrrolidone (PVP), in combination with one or more blocks another biocompatible or biodegradable polymer that comprises lactide, glycolide, caprolactone, or a combination thereof.

In specific aspects, the biodegradable polymer can comprise one or more lactide residues. To that end, the polymer can comprise any lactide residue, including all racemic and stereospecific forms of lactide, including, but not limited to, L-lactide, D-lactide, and D,L-lactide, or a mixture thereof. Useful polymers comprising lactide include, but are not limited to poly(L-lactide), poly(D-lactide), and poly(DL-lactide); and poly(lactide-co-glycolide), including poly(L-lactide-co-glycolide), poly(D-lactide-co-glycolide), and poly(DL-lactide-co-glycolide); or copolymers, terpolymers, combinations, or blends thereof. Lactide/glycolide polymers can be conveniently made by melt polymerization through ring opening of lactide and glycolide monomers.

Additionally, racemic DL-lactide, L-lactide, and D-lactide polymers are commercially available. The L-polymers are more crystalline and resorb slower than DL-polymers. In addition to copolymers comprising glycolide and DL-lactide or L-lactide, copolymers of L-lactide and DL-lactide are commercially available. Homopolymers of lactide or glycolide are also commercially available. In some embodiments, the polymer is poly(DL-lactide-co-glycolide).

When the biodegradable polymer is poly(lactide-co-glycolide), poly(lactide), or poly(glycolide), the amount of lactide and glycolide in the polymer can vary, for example the biodegradable polymer can be poly(lactide), 95:5 poly(lactide-co-glycolide) 85:15 poly(lactide-co-glycolide), 75:25 poly(lactide-co-glycolide), 65:35 poly(lactide-co-glycolide), or 50:50 poly(lactide-co-glycolide), where the ratios are mole ratios.

The polymer can be a poly(caprolactone) or a poly(lactide-co-caprolactone). In one aspect, the polymer can be a poly(lactide-caprolactone), which, in various aspects, can be 95:5 poly(lactide-co-caprolactone), 85:15 poly(lactide-co-caprolactone), 75:25 poly(lactide-co-caprolactone), 65:35 poly(lactide-co-caprolactone), or 50:50 poly(lactide-co-caprolactone), where the ratios are mole ratios.

In some embodiments, the microparticle includes about at least 90 percent hydrophobic polymer and about not more than 10 percent hydrophilic polymer. Examples of hydrophobic polymers include polyesters such as poly lactic acid (PLA), polyglycolic acid (PGA), poly(D,L-lactide-co-glycolide)(PLGA), and poly D,L-lactic acid (PDLLA); polycaprolactone; polyanhydrides, such as polysebacic anhydride, poly(maleic anhydride); and copolymers thereof. Examples of hydrophilic polymers include poly(alkylene glycols) such as polyethylene glycol (PEG), polyethylene oxide (PEO), and poly(ethylene glycol) amine; polysaccharides; poly(vinyl alcohol) (PVA); polypyrrolidone; polyacrylamide (PAM); polyethylenimine (PEI); poly(acrylic acid); poly(vinylpyrolidone) (PVP); or a copolymer thereof.

In some embodiments, the microparticle includes about at least 85 percent hydrophobic polymer and at most 15 percent hydrophilic polymer.

In some embodiments, the microparticle includes about at least 80 percent hydrophobic polymer and at most 20 percent hydrophilic polymer.

In some embodiments, the microparticle includes PLA. In some embodiments, the PLA is acid-capped. In some embodiments, the PLA is ester-capped.

In some embodiments, the microparticle includes PLA and PLGA-PEG.

In some embodiments, the microparticle includes PLA and PLGA-PEG and PVA.

In some embodiments, the microparticle includes PLA, PLGA, and PLGA-PEG.

In some embodiments, the microparticle includes PLA, PLGA, and PLGA-PEG and PVA.

In some embodiments, the microparticle includes PLGA.

In some embodiments, the microparticle includes a copolymer of PLGA and PEG.

In some embodiments, the microparticle includes a copolymer of PLA and PEG.

In some embodiments, the microparticle comprises PLGA and PLGA-PEG, and combinations thereof.

In some embodiments, the microparticle comprises PLA and PLA-PEG.

In some embodiments, the microparticle includes PVA.

In some embodiments, the microparticles include PLGA, PLGA-PEG, PVA, or combinations thereof.

In some embodiments, the microparticles include the biocompatible polymers PLA, PLA-PEG, PVA, or combinations thereof.

It is understood that any combination of the aforementioned biodegradable polymers can be used, including, but not limited to, copolymers thereof, mixtures thereof, or blends thereof. Likewise, it is understood that when a residue of a biodegradable polymer is disclosed, any suitable polymer, copolymer, mixture, or blend, that comprises the disclosed residue, is also considered disclosed. To that end, when multiple residues are individually disclosed (i.e., not in combination with another), it is understood that any combination of the individual residues can be used.

Non-limiting examples of commercially available polymers useful for the production of microparticles according to the present invention include Boeringer Inglehiem produced suitable polymers under the designations R 202H, RG 502, RG 502H, RG 503, RG 503H, RG 752, RG 752H, RG 756 and others. LH-RH microparticles with R202H, RG752H, or RG503H Resomer RG752H, Purasorb PDL 02A, Purasorb PDL 02, Purasorb PDL 04, Purasorb PDL 04A, Purasorb PDL 05, Purasorb PDL 05A Purasorb PDL 20, Purasorb PDL 20A; Purasorb PG 20; Purasorb PDLG 5004, Purasorb PDLG 5002, Purasorb PDLG 7502, Purasorb PDLG 5004A, Purasorb PDLG 5002A, Resomer RG755S, Resomer RG503, Resomer RG502, Resomer RG503H, Resomer RG502H, Resomer RG752, Resomer 7525 DLG 4A 75:25 polyor any combination thereof.

One consideration in selecting a preferred polymer is the hydrophilicity/hydrophobicity of the polymer. Both polymers and active agents may be hydrophobic or hydrophilic. Where possible it is desirable to select a hydrophilic polymer for use with a hydrophilic active agent, and a hydrophobic polymer for use with a hydrophobic active agent.

Continuous and Dispersed Phase Solvents

Solvents for the active agent will vary depending upon the nature of the active agent. Typical solvents that may be used in the dispersed phase to dissolve the active agent include, but are not limited to, water, methanol, ethanol, dimethyl sulfoxide (DMSO), dimethyl formamide, dimethyl acetamide, dioxane, tetrahydrofuran (THF), dichloromethane (DCM), ethylene chloride, carbon tetrachloride, chloroform, lower alkyl ethers such diethyl ether and methyl ethyl ether, hexane, cyclohexane, benzene, acetone, ethyl acetate, methyl ethyl ketone, acetic acid, or mixtures thereof. Additionally, an acid such as glacial acetic acid, lactic acid, or fatty acids or acrylic acid may be used in the process to help improve the solubility and encapsulation of the active agent in the polymer. Selection of suitable solvents for a given system will be within the skill in the art in view of the instant disclosure.

The continuous phase may comprise any liquid in which the polymer is substantially insoluble. Suitable liquids may include, for example, water, methanol, ethanol, propanol (e.g. 1-propanol, 2-propanol), butanol (e.g. 1-butanol, 2-butanol or tert-butanol), pentanol, hexanol, heptanol, octanol and higher alcohols; diethyl ether, methyl tert butyl ether, dimethyl ether, dibutyl ether, simple hydrocarbons, including pentane, cyclopentane, hexane, cyclohexane, heptane, cycloheptane, octane, cyclooctane and higher hydrocarbons. If desired, a mixture of liquids may be used.

The continuous phase can be water, optionally with one or more surface active agents, for example, alcohols, such as methanol, ethanol, propanol (e.g. 1-propanol, 2-propanol), butanol (e.g. 1-butanol, 2-butanol or tert-butanol), isopropyl alcohol, Polysorbate 20, Polysorbate 40, Polysorbate 60 and Polysorbate 80. Surface active agents, such as alcohols, reduce the surface tension of the second liquid receiving the droplets, which reduces the deformation of the droplets when they impact the second liquid, thus decreasing the likelihood of non-spherical droplets forming. This is particularly important when the extraction of solvent from the droplet is rapid. If the continuous phase water and one or more surface active agents, the continuous phase may comprise a surface active agent content of from 1 to 95% v/v, optionally from 1 to 30% v/v, optionally from 1 to 25% v/v, further optionally from 5% to 20% v/v and further more optionally from 10 to 20% v/v. The % volume of surface active agent is calculated relative to the volume of the continuous phase.

Frequently, the continuous phase will also contain surfactant, stabilizers, salts, or other additives that modify or effect the emulsification process. Typical surfactants include sodium dodecyl sulphate, dioctyl sodium sulfo succinate, span, polysorbate 80, tween 80, pluronics and the like. Particular stabilizers include talc, PVA and colloidal magnesium hydroxide. Viscosity boosters include polyacrylamide, carboxymethyl cellulose, hydroxymethyl cellulose, methyl cellulose and the like. Buffer salts can be used as drug stabilizers and even common salt can be used to help prevent migration of the active agent into the continuous phase. One problem associated with salt saturation of the continuous phase is that PVA and other stabilizers may have a tendency to precipitate as solids from the continuous phase. In such instances a particulate stabilizer might be used. Suitable salts, such as sodium chloride, sodium sulfate and the like, and other additives would be apparent to those of ordinary skill in the art in view of the instant disclosure.

In some embodiments, the continuous phase includes from 50-100% water. The aqueous continuous phase may include a stabilizer. A preferred stabilizer is polyvinyl alcohol (PVA) in an amount of from about 0.1% to about 5.0%. Other stabilizers suitable for use in the continuous phase 14 would be apparent to those of ordinary skill in the art in view of the instant disclosure.

Surface Treatment

A surface treatment may be applied to facilitate the aggregation of the formed microparticles upon medical use, for example to form an implant-like depot in the vitreous of the eye upon intravitreal injection. Examples of surface-treated microparticles are disclosed in Application No. US 2017-0135960 and Application No. US 2018-0326078 assigned to Graybug Vision, Inc., which are specifically incorporated by reference.

The surface treatment causes the particles to fuse together at temperatures around 37° C. by lowering the Tg (glass transition temperature) of the polymers on the surface. Without wishing to be bound to any one theory, the surface-treatment solution induces hydrolysis of the polymers on the surface, lowering the molecular weight and therefore lowering the Tg of the polymers to a temperature below the temperature of the vitreous (Qutachi et al. Acta Biomater. 2014, 10:5090-5098). The reduction in Tg, which is limited to the surface of the microparticles, allows the microparticles to cross-link with neighboring particles and form an aggregate upon intravitreal injection. After intravitreal injection, the microparticles degrade. For example, PLGA has a Tg of approximately 50° C., so at vitreous temperatures of around 35° C., the formed microparticles should remain solid and not transition into malleable structures. The surface-treatment, however, lowers the Tg of the polymers on the surface, which allows the microparticles to aggregate at the temperature of the vitreous.

In some embodiments, the surface treatment includes treating microparticles with aqueous base, for example, sodium hydroxide and a solvent (such as an alcohol, for example ethanol or methanol, or an organic solvent such as DMF, DMSO or ethyl acetate) as otherwise described above. More generally, a hydroxide base is used, for example, potassium hydroxide. An organic base can also be used. In other embodiments, the surface treatment as described above is carried out in aqueous acid, for example hydrochloric acid. In some embodiments, the surface treatment includes treating microparticles with phosphate buffered saline and ethanol. In some embodiments the surface treatment can be conducted with an organic solvent. In some embodiments the surface treatment can be conducted with ethanol. In other various embodiments, the surface treatment is carried out in a solvent selected from methanol, ethyl acetate and ethanol. Non-limiting examples are ethanol with an aqueous organic base; ethanol and aqueous inorganic base; ethanol and sodium hydroxide; ethanol and potassium hydroxide; an aqueous acidic solution in ethanol; aqueous hydrochloric acid in ethanol; and aqueous potassium chloride in ethanol.

In some embodiments, the surface treatment is carried out at a temperature of not more than 5, 6, 7, 8, 9, 10, 11, 12, 13, 14, 15, 16, 17 or 18° C. at a reduced temperature of about 5 to about 18° C., about 5 to about 16° C., about 5 to about 15° C., about 0 to about 10° C., about 0 to about 8° C., or about 1 to about 5° C., about 5 to about 20° C., about 1 to about 10° C., about 0 to about 15° C., about 0 to about 10° C., about 1 to about 8° C., or about 1 to about 5° C. Each combination of each of these conditions is considered independently disclosed as if each combination were separately listed. To assist with maintenance of the necessary temperatures to allow for surface treatment of the microparticles, the plug flow reactor may be optionally jacketed.

The pH of the surface treatment will of course vary based on whether the treatment is carried out in basic, neutral or acidic conditions. When carrying out the treatment in base, the pH may range from about 7.5 to about 14, including not more than about 8, 9, 10, 11, 12, 13 or 14. When carrying out the treatment in acid, the pH may range from about 6.5 to about 1, including not less than 1, 2, 3, 4, 5, or 6. When carrying out under neutral conditions, the pH may typically range from about 6.4 or 6.5 to about 7.4 or 7.5. The surface treatment can be carried out at any pH that achieves the desired purpose. Non-limiting examples of the pH are between about 6 and about 8, 6.5 and about 7.5, about 1 and about 4; about 4 and about 6; and 6 and about 8. In some embodiments the surface treatment can be conducted at a pH between about 8 and about 10. In some embodiments the surface treatment can be conducted at a pH between about 10.0 and about 13.0. In some embodiments the surface treatment can be conducted at a pH between about 12 and about 14.

A key aspect is that the treatment, whether done in basic, neutral or acidic conditions, includes a selection of the combination of the time, temperature, pH agent and solvent that causes a mild treatment that does not significantly damage the particle in a manner that forms pores, holes or channels. Each combination of each of these conditions is considered independently disclosed as if each combination were separately listed.

In some embodiments, the surface treatment includes treating microparticles with an aqueous solution of pH=6.6 to 7.4 or 7.5 and ethanol at a reduced temperature of about 1 to about 10° C., about 1 to about 15° C., about 5 to about 15° C., or about 0 to about 5° C. In some embodiments, the surface treatment includes treating microparticles with an aqueous solution of pH=6.6 to 7.4 or 7.5 and an organic solvent at a reduced temperature of about 0 to about 10° C., about 5 to about 8° C., or about 0 to about 5° C. In some embodiments, the surface treatment includes treating microparticles with an aqueous solution of pH=1 to 6.6 and ethanol at a reduced temperature of about 0 to about 10° C., about 0 to about 8° C., or about 0 to about 5° C. In some embodiments, the surface treatment includes treating microparticles with an organic solvent at a reduced temperature of about 0 to about 18° C., about 0 to about 16° C., about 0 to about 15° C., about 0 to about 10° C., about 0 to about 8° C., or about 0 to about 5° C. The decreased temperature of processing (less than room temperature, and typically less than 18° C.) assists to ensure that the particles are only “mildly” surface treated.

In certain embodiments, the microparticles are surface-treated with approximately 0.0075 M NaOH/ethanol to 0.75 M NaOH/ethanol (30:70, v:v).

In certain embodiments, the microparticles are surface-treated with approximately 0.75 M NaOH/ethanol to 2.5 M NaOH/ethanol (30:70, v:v).

In certain embodiments, the microparticles are surface-treated with approximately 0.0075 M HCl/ethanol to 0.75 M NaOH/ethanol (30:70, v:v).

In certain embodiments, the microparticles are surface-treated with approximately 0.75 M NaOH/ethanol to 2.5 M HCl/ethanol (30:70, v:v).

### EXAMPLES OF THE PRESENT INVENTION

### Example 1. Synthesis of Risperidone-Containing Microparticles Using Plug Flow Reactor and TWHFTFF

Dispersed phase is prepared by mixing a 180 mg/mL solution of polylactic-co-glycolic acid (PLGA)/monomethoxy polyethylene glycol-PLGA (mPEG) (99:1 mixture) in dichloromethane (DCM) with a 50.1 mg/mL solution of risperidone in dimethylsulfoxide (DMSO) in the dispersed phase tank until a homogenous solution is achieved. Continuous phase is prepared from 0.25% PVA and water in the continuous phase tank. The dispersed phase and the continuous phase are fed through their respective conduits into the in-line mixer. The dispersed phase is passed through a hydrophobic PTFE filter and fed into the in-line mixer at a rate of 20 mL/min via conduit. The continuous phase is passed through a hydrophilic PVDF filter (0.20 μm) and fed into the in-line mixer at a rate of 2000 mL/min via conduit. An impeller in the in-line mixer rotating at 4000 rpm provides sufficient mixing of the dispersed phase and continuous phase to provide an emulsion. The emulsion exits the in-line mixer and enters the plug flow reactor (0.5 inch diameter by 7 meter length) at a flow rate of 2020 mL/min. Sterile water is added to the plug flow reactor upon entry of the emulsion at a flow rate of 4040 mL/min at the solvent extraction phase inlet approximately 5 cm along the plug flow reactor distal to the mixer inlet. The emulsion traverses the plug flow reactor for a 20 second residence time within which microparticles are formed. The resulting suspension exits the plug flow reactor into a thick wall hollow fiber tangential flow filter with a 8 μm membrane pore size. The permeate is removed through the filter at a flow rate of 3000 mL/min into a solvent waste tank. The retentate exits the filter at a flow rate of 2060 mL/min into the holding tank to provide a filtered solution of risperidone-containing microparticles.

### Example 2. Synthesis of Risperidone-Containing Microparticles Using Continuous Centrifugation

Dispersed phase is prepared by mixing a 180 mg/mL solution of polylactic-co-glycolic acid (PLGA)/monomethoxy polyethylene glycol-PLGA (mPEG) (99:1 mixture) in dichloromethane (DCM) with a 50.1 mg/mL solution of risperidone in dimethylsulfoxide (DMSO) in the dispersed phase tank until a homogenous solution is achieved. Continuous phase is prepared from 0.25% PVA and water in the continuous phase tank. The dispersed phase and the continuous phase are fed through their respective conduits into the in-line mixer. The dispersed phase is passed through a hydrophobic PTFE filter and fed into the in-line mixer at a rate of 20 mL/min via conduit. The continuous phase is passed through a hydrophilic PVDF filter (0.20 μm) and fed into the in-line mixer at a rate of 2000 mL/min via conduit. An impeller in the in-line mixer rotating at 4000 rpm provides sufficient mixing of the dispersed phase and continuous phase to provide an emulsion. The emulsion exits the in-line mixer and enters the plug flow reactor (0.5 inch diameter by 7 meter length) at a flow rate of 2020 mL/min. Sterile water is added to the plug flow reactor upon entry of the emulsion at a flow rate of 4040 mL/min at the solvent extraction phase inlet approximately 5 cm along the plug flow reactor distal to the mixer inlet. The emulsion traverses the plug flow reactor for a 20 second residence time within which microparticles are formed. The resulting suspension exits the plug flow reactor into an in-line continuous centrifuge rotating at 2000 rpm. The supernatant is removed at a flow rate of 6000 mL/min into a solvent waste tank. The concentrated slurry exits the filter into the receiving tank to provide a purified slurry of risperidone-containing microparticles.

### Example 3. Continuous Centrifugation as a Separation Process to Remove Small Particles

Continuous centrifugation was incorporated in the production of surface treated particles (STP) as a separation process in order to remove to small particles as well as to wash and concentrate the particles. This process separates out small particles continuously from the larger particles by centrifugation and discharges the retained larger particles at the end of the cycle. The continuous centrifugation was performed with the UniFuge Pilot separation system from Pneumatic Scale Angelus. FIG. 1M and FIG. 1N refer to Centrifuge 1, Centrifuge 2, Centrifuge 3, and Centrifuge 4.

Centrifuge 1 occurs concurrently with a homogenization step for approximately 2 hours for a 200 g scale batch: as the dispersed phase (DP) and continuous phase (CP) were mixed in homogenizer, the resulting liquid coming out of the homogenizer flowed into a glass vessel. The vessel's volume is much less than the total liquid volume that was processed during the homogenizer during hours of formulation, so as the CP/DP entered the glass vessel at certain flow rate, the centrifuge started to pump the liquid out of the vessel at the same flow rate. The centrifuge kept spinning the supernatant out as more liquid was pumped in. A small volume of concentrated particles were retained in the centrifuge bowl (˜1-2 L), but the large amount of liquid with smaller particles (hundreds of liters) were removed as the supernatant, resulting in a size reduction from pre-centrifuge sample to centrifuge 1 sample (FIG. 1M). (Centrifuge 1 sample is the retained sample after centrifuge 1 process).

Centrifuge 2 is the centrifuge process involved in the first wash cycle after the homogenization step, when appropriately-sized particles were previously retained in the centrifuge bowl in a high concentration. The concentrated particles from the centrifuge are pumped back into the glass vessel and diluted to the appropriate volume that vessel can hold (i.e., 10 L). The suspension is then pumped to the centrifuge again and concentrated down to 1-2 L. In this process, ˜8-9 L of wash liquid containing small particles was removed, resulting in a size reduction in <10 um range from centrifuge 1 to centrifuge 2 as shown in FIG. 1M.

Centrifuge 3-4 are two additional wash cycles that are similar to Centrifuge 2.

Continuous centrifugation effectively removed small particles. For example, before any centrifugation, particles less than 10 μm comprised 6.8% of the total particle size distribution (FIG. 2I). The percent of particles less than 10 μm was decreased by 21% after only one round of centrifugation. The fraction of small particles was further reduced with subsequent centrifugation and after three rounds particles less than 10 μm comprised only 2.7% of the total particles. This corresponded to a 60% reduction in the percent of particles less than 10 μm compared with no centrifugation.

The particle size of the supernatant removed by each round of centrifugation (FIG. 2J) showed the effectiveness of small particle removal in each centrifugation round.

During production, particles were washed again with the continuous centrifugation system (three wash cycles similar to Centrifuge 2-4) following surface treatment, which can further reduce the fraction of small particles. As can be seen in FIG. 2K, the amount of small particles less than 10 μm in the final product was 69% lower than that immediately following homogenization and prior to any centrifugation. This is also reflected in the shift in the d10 size from 11.6 μm before centrifugation to 15.30 μm in the final product.

After this step, there is also a sieving step (not shown). In the sieving step, the centrifuge pulls the diluted suspension through a 50 μm filter and concentrates the particle suspension again in the centrifuge bowl, removing >50 μm particulates.

### Example 4. Production of Risperidone-Containing Microparticles Using a Microfluidic Droplet Generator and a Plug Flow Reactor

A polymer solution is prepared by combining a mixture of polylactic-co-glycolic acid (PLGA) and monomethoxy polyethylene glycol (mPEG) (99% PLGA, 1% mPEG) dissolved in DCM to obtain a 180 mg/mL solution. The solution is mixed at ambient temperature with a stir bar on a stir plate until the polymers are dissolved. The risperidone solution is prepared by dissolving risperidone in DMSO. The solution is mixed at ambient temperature with a stir bar on a stir plate until risperidone is completely dissolved. The dispersed phase is prepared by combining the polymer solution with the risperidone solution and mixing on a stir plate to achieve a homogeneous solution. The dispersed phase is sterile filtered into an intermediate sterile container (disperse phase holding vessel) and later pumped into the in-line mixer. A hydrophobic PTFE filter is used for dispersed phase filtration. The continuous phase solution consists of 0.0025 g/g polyvinyl alcohol (0.25% PVA) and 1×PBS buffer solution in water. The continuous phase is produced by dispersing PVA powder in ambient temperature water-for-injection (WFI) while mixing and then heating to at least 80° C. The PVA is dissolved by mixing at 80-90° C. for 1 hour. The solution is then cooled to ambient temperature. A clarification step recirculates the solution through a filter to remove any undissolved PVA. Typically, a hydrophilic PVDF capsule filter is used. The CP is sterile filtered directly into the in-line mixer used for microsphere formulation. Typically, a hydrophilic PVDF capsule filter is used.

Microparticles are formed by combining the CP and DP into a flow-focusing microfluidic droplet generating device, such as Dololmite Telos® High-Throughout Droplet System. The microparticles are highly monodisperse and do not require downstream filtration. The microparticles, however, are not yet sufficiently solid to be filterable immediately and to aid in solidification, the microparticle suspension produced in the droplet generator is flowed through a plug flow reactor where solvent extraction phase and surface treatment solution are added serially along the plug flow reactor in order to extract solvent and surface treat, respectively. The microparticle suspension produced in the droplet generator and plug flow reactor is received into the dilution vessel. Sterile filtered ambient WFI is added to the dilution vessel and the suspension is diluted to the target filling concentration.

### Example 5. Production of Risperidone-Containing Microparticles Using Continuous Centrifugation and TWHFTFF

Dispersed phase is prepared by mixing a 180 mg/mL solution of polylactic-co-glycolic acid (PLGA)/monomethoxy polyethylene glycol-PLGA (mPEG) (99:1 mixture) in dichloromethane (DCM) with a 50.1 mg/mL solution of risperidone in dimethylsulfoxide (DMSO) in the dispersed phase tank until a homogenous solution is achieved. Continuous phase is prepared from 0.25% PVA and water in the continuous phase tank. The dispersed phase and the continuous phase are fed through their respective conduits into the in-line mixer. The dispersed phase is passed through a hydrophobic PTFE filter and fed into the in-line mixer at a rate of 20 mL/min via conduit. The continuous phase is passed through a hydrophilic PVDF filter (0.20 μm) and fed into the in-line mixer at a rate of 2000 mL/min via conduit. An impeller in the in-line mixer rotating at 4000 rpm provides sufficient mixing of the dispersed phase and continuous phase to provide an emulsion. The emulsion exits the in-line mixer and enters a quench vessel at a flow rate of 2020 mL/min. Sterile water is added to the plug flow reactor upon entry of the emulsion at a flow rate of 4040 mL/min at the solvent extraction phase inlet approximately 5 cm along the plug flow reactor distal to the mixer inlet to afford a liquid dispersion containing the microparticles. The liquid dispersion is then transferred to a centrifuge to form a concentrated slurry. The concentrated slurry is then recirculated to the quench vessel. In some embodiments, prior to the recirculation, the quench vessel is filled with water. In an alternative embodiment, the concentrated slurry reenters the quench vessel and water is simultaneously added to the quench vessel. The resulting liquid dispersion is then retransferred to the centrifuge to once again form a concentrated slurry. In some embodiments, the concentrated slurry is recirculated to the quench vessel and washed once more. In some embodiments, the concentrated slurry is recirculated to the quench vessel and washed twice more. In some embodiments, the concentrated slurry is further surface-treated by adding surface treatment phase to the liquid dispersion in the quench vessel following one, two, or three washes with water. Following surface treatment, the liquid dispersion is centrifuged and the resulting concentrated slurry is transferred to a second quench vessel that is directly transferred to a thick wall hollow fiber tangential flow filter with a 8 μm membrane pore size. The permeate is removed through the filter into a solvent waste tank. The retentate exits the filter into the holding tank to provide a filtered solution of risperidone-containing microparticles.

### Example 6. Non-Limiting Example of a Microparticle Process of the Present Invention

A ViaFuge Centrifuge is started under fill mode at 1000 rpm±10 rpm and primed with water at approximately 3 LPM until full. The in-line CP filter, Silverson in-line assembly and all tubing leading up to quench vessel 1 with continuous phase (CP) at 2 LPM is also primed. Quench vessel 1 is filled up to 10±1 L with CP at 3 LPM and set at 200±5 rpm counter-clockwise (CCW) so the liquid is up-pumping. When the quench vessel liquid level has reached 10±1 L, the ViaFuge setting is changed from fill mode to process mode, which ramps the ViaFuge to 2000±10 rpm. Quench vessel 1 contents are pumped to the ViaFuge at 3 LPM while continuing to fill FR-1 with CP at 3 LPM. The Silverson set speed is increased to 3600±10 rpm and once the CP flow is stable and the Silverson outlet line is free of air bubbles, the dispersed phase (DP) pump line is started at 12.5 mL/min. CP is pumped at 3 LPM and DP is pumped at 12.5 mL/min and this process is continued until the DP bottle is empty and the DP pump is stopped. When the CP/DP inlet tubing into quench vessel 1 is clear of particles, the Silverson homogenizer is reduced to 0 rpm and the CP pump is stopped. When quench vessel 1 is empty, the outlet flow from quench vessel 1 is stopped by stopping the ViaFuge inlet pump. The ViaFuge is then stopped. Connect quench vessel 1, quench vessel 2, and the ViaFuge to the chiller set at 5° C. The quench vessel 1 bottom valve is opened and the residual liquid from quench vessel 1 is drained into a waste container. The bottom valve is closed. Quench vessel 1 is filled with water at 3 LPM to a volume of 5±1 L and set the quench vessel 1 mixer speed to 150±5 rpm. The retained microparticles are discharged from the ViaFuge to quench vessel 1 at 1 LPM. The ViaFuge is started under fill mode at 1000±10 rpm and filled with water at 3 LPM until full and then stopped. Any additional retained microparticles are discharged from the ViaFuge to quench vessel 1 at 1 LPM. The ViaFuge is again started under fill mode at 1000±10 rpm and filled with water at 3 LPM until full and then stopped. Any additional retained microparticles are again discharged from the ViaFuge to quench vessel 1 at 1 LPM. The ViaFuge is again started under fill mode at 1000±10 rpm and filled with water at 3 LPM until full. The ViaFuge setting is changed from fill mode to process mode, which ramps the ViaFuge to 2000±10 rpm and the quench vessel 1 contents are pumped to the ViaFuge at 2 LPM until quench vessel 1 is empty and the ViaFuge is stopped.

Quench vessel 1 is again filled with water at 3 LPM to a volume of 8.5±1 L. The retained microparticles are discharged from the ViaFuge to quench vessel 1 at 1 LPM. The ViaFuge is started under fill mode at 1000±10 rpm and the Viafuge is filled with water at 3 LPM until full. The ViaFuge setting is changed from fill mode to process mode, which ramps the ViaFuge to 2000±10 rpm and the quench vessel contents are pumped to the ViaFuge at 2 LPM until quench vessel 1 is empty and the ViaFuge is stopped. This process is repeated three times.

The bottom valve of quench vessel 1 is opened and quench vessel 1 liquid is pumped from the bottom valve of quench vessel 1 at no more than 1 LPM until all the liquid is removed from quench vessel 1. When all the liquid is removed from the quench vessel, the waste pump is stopped and the bottom valve of the quench vessel is closed. The chiller setpoint is set at 5° C. and the quench vessel mixer speed is set to 150±5 rpm. The quench vessel 1 water input connection is switched from the ambient water drum to the cold water drum. Connect the upstream end of the PureWeld® XL pump tubing to the dip tube port of the 7 L jacketed glass vessel with the ST solution that is less than or equal to a temperature of 8° C. Connect the downstream end of the pump tubing to the CP/DP/ST inlet dip tube of quench vessel 1. Pump 5 L of ST solution from the 7 L jacketed vessel to quench vessel at 3 LPM. After 30±0.5 minutes of surface treatment, quench vessel 1 is filled with cold water at 3 LPM to a volume of 10±1 L. The ViaFuge is started under fill mode at 1000±10 rpm and the ViaFuge is filled with cold water at 3 LPM until full. The ViaFuge setting is changed from fill mode to process mode, which ramps the ViaFuge to 2000±10 rpm and the quench vessel contents are pumped to the ViaFuge at 2 LPM until quench vessel 1 is empty and the ViaFuge is stopped.

The bottom valve of quench vessel 1 is opened and the quench vessel liquid waste from the bottom valve is pumped at no more than 1 LPM until all the liquid is removed from quench vessel 1. When all the liquid is removed from quench vessel 1, the waste pump is stopped and the bottom valve of the quench vessel is closed. The quench vessel 1 is filled with cold water at 3 LPM to a volume of 5±1 L and the mixer speed is set to 150±5 rpm. The retained microparticles from the ViaFuge are discharged to quench vessel 1 at 1 LPM. The ViaFuge is started under fill mode at 1000±10 rpm and filled with cold water at 3 LPM until full and stopped. This recirculation process is repeated four times.

The quench vessel 1 is filled with cold water at 3 LPM to a volume of 8.5±1 L. The retained microparticles from the ViaFuge are discharged to quench vessel 1 at 1 LPM. The ViaFuge is started under fill mode at 1000±10 rpm and filled with cold water at 3 LPM until full. The ViaFuge setting is changed from fill mode to process mode, which ramps the ViaFuge to 2000±10 rpm. The quench vessel 1 contents are pumped to the ViaFuge at 2 LPM until the volume in quench vessel 1 is reduced to ˜2 L. When the volume in quench vessel 1 is at ˜2 L, while continuing to run the ViaFuge in process mode and ViaFuge pump at 2 LPM, cold water is added to quench vessel 1 at 2 LPM to dilute the suspension and collect as much of the particles out of quench vessel 1 as possible. Water is added for a minimum of 5 minutes. The ViaFuge is run in process mode at 2000±10 rpm and quench vessel 1 contents are pumped to the ViaFuge at 2 LPM until quench vessel 1 is empty and the ViaFuge is stopped.

The direction of the ViaFuge ball valve is changed from quench vessel 1 to quench vessel 2 and the direction of the cold water ball valve is changed from quench vessel 1 to quench vessel 2. With the bottom valve of quench vessel 2 open, quench vessel 2 is filled with cold water at 3 LPM until all the air is purged below the filter. The bottom valve is closed and quench vessel 2 is filled to a volume of 5±1 L. The quench vessel 2 mixer speed is set to 200±5 rpm. The retained microparticles from the ViaFuge are discharged to quench vessel 1 at 1 LPM. The ViaFuge is started under fill mode at 1000±10 rpm and filled with cold water at 3 LPM until full and stopped. This recirculation process is repeated three times. The ViaFuge setting is changed from fill mode to process mode, which ramps up the ViaFuge to 2000±10 rpm. Quench vessel 2 contents are pumped through the 50 micron bottom filter of quench vessel 2 to the ViaFuge at 2 LPM. While continuing to run the ViaFuge in process mode and ViaFuge pump at 2 LPM, cold water is added to quench vessel at 2 LPM to continually dilute the suspension in quench vessel 2. Cold water is added for a minimum of 10 minutes. The ViaFuge is run in process mode at 2000±10 rpm and quench vessel 2 contents are pumped to the ViaFuge at 2 LPM until quench vessel 2 volume is reduced to ˜2 L. The ViaFuge pump is stopped. Quench vessel 2 is filled with cold water at 4 LPM to a volume of 10±1 L. Quench vessel 2 contents are pumped to the ViaFuge at 2 LPM and the ViaFuge is continued in process mode at 2000±10 rpm until quench vessel 2 is empty. The ViaFuge is stopped and the concentrated slurry is transferred to a holding tank for further processing.

This specification has been described with reference to embodiments of the invention. However, one of ordinary skill in the art appreciates that various modifications and changes can be made without departing from the scope of the invention as set forth herein. Accordingly, the specification is to be regarded in an illustrative rather than a restrictive sense, and all such modifications are intended to be included within the scope of invention.

We claim:

1. A process of producing drug-loaded microparticles in a continuous process comprising:
a) continuously forming an emulsion comprising a dispersed phase and a continuous phase in a mixer, wherein the dispersed phase comprises a drug, a polymer, and at least one solvent;
b) directly feeding the emulsion into a quench vessel, whereupon entering the quench vessel the emulsion is mixed with an extraction phase to form a liquid dispersion, wherein a portion of the solvent is extracted into the extraction phase and microparticles are formed;
c) continuously feeding the liquid dispersion from the quench vessel into a parallel bank of centrifuges via an outlet from the quench vessel, wherein a portion of the liquid dispersion containing solvent and microparticles below a specific size threshold are removed with a waste solvent liquid and remaining microparticles above the specified size threshold are isolated as a concentrated slurry; and
d) transferring the concentrated slurry from the centrifuge to a receiving vessel.

2. The process of claim 1, further comprising transferring the concentrated slurry in step (d) from the receiving vessel to a thick wall hollow fiber tangential flow filter, wherein the thick wall hollow fiber tangential flow filter is in direct fluid communication with the receiving vessel, wherein the tangential flow depth flow filter has a pore size of greater than 1 μm, and wherein a portion of the liquid dispersion containing solvent and microparticles below a specified-size threshold are removed as a permeate.

3. The process of claim 1, wherein the liquid dispersion from the outlet of the quench vessel is diverted to a first centrifuge in the parallel bank of centrifuges and then is diverted to one or more additional centrifuges in the parallel bank of centrifuges after a set centrifugation time.

4. The process of claim 1, wherein the liquid dispersion from the outlet of the quench vessel is run through two or more centrifuges operating simultaneously in the parallel bank of centrifuges.

5. The process of claim 1, wherein the centrifuge is a filtration centrifuge.

6. The process of claim 1, wherein the centrifuge is a sedimentation centrifuge.

7. The process of claim 1, wherein the concentrated slurry in the receiving vessel is diluted with a wash phase and returned to the parallel bank of centrifuges for additional processing.

8. The process of claim 1, further comprising adding a surface treatment phase to the quench vessel in step b) distal from the addition of the extraction phase.

9. The process of claim 1, further comprising adding a surface treatment phase to the receiving vessel following step d).

10. A process of producing drug-loaded microparticles in a continuous process comprising:
a) continuously forming an emulsion comprising a dispersed phase and a continuous phase in a mixer, wherein the dispersed phase comprises a drug, a polymer, and at least one solvent;
b) directly feeding the emulsion into a quench vessel, whereupon entering the quench vessel the emulsion is mixed with an extraction phase to form a liquid dispersion, wherein a portion of the solvent is extracted into the extraction phase and microparticles are formed;
c) continuously feeding the liquid dispersion from the quench vessel into a continuous liquid centrifuge via an outlet from the quench vessel, wherein a portion of the liquid dispersion containing solvent and microparticles below a specific size threshold are removed with a waste solvent liquid and remaining microparticles above the specified size threshold are isolated as a concentrated slurry; and
d) transferring the concentrated slurry from the centrifuge to a receiving vessel.

11. The process of claim 10, wherein the continuous liquid centrifuge is a solid bowl centrifuge.

12. The process of claim 10, wherein the continuous liquid centrifuge is a conical plate centrifuge.

13. The process of claim 10, further comprising washing the concentrated slurry in step (d) in the receiving vessel to afford a liquid dispersion that is transferred to a thick wall hollow fiber tangential flow filter, wherein the thick wall hollow fiber tangential flow filter is in direct fluid communication with the receiving vessel, wherein the tangential flow depth flow filter has a pore size of greater than 1 μm, and wherein a portion of the liquid dispersion containing solvent and microparticles below a specified-size threshold are removed as a permeate and the retentate is transferred to a reactor vessel.

14. The process of claim 13, further comprising filtering the retentate through a filter in the reactor vessel and transferring the retentate back to the thick wall hollow fiber tangential flow filter via a loop circuit between the thick wall hollow fiber tangential flow filter and the reactor vessel.

15. The process of claim 14, where the filter is a 50 μm filter.

16. The process of claim 10, wherein the concentrated slurry in the receiving vessel is diluted with a wash phase and returned to the continuous liquid centrifuge for additional processing.

17. The process of claim 10, further comprising a surface treatment phase to the quench vessel in step b) distal from the addition of the extraction phase.

18. The process of claim 10, further comprising adding a surface treatment phase to the receiving vessel following step d).

19. A process of continuously producing a drug-loaded polymeric microparticle comprising:
a) continuously forming an emulsion comprising a dispersed phase and a continuous phase in a mixer, wherein the dispersed phase comprises a drug, a polymer, and at least one solvent;
b) directly feeding the emulsion into a plug flow reactor, wherein upon entering the plug flow reactor, the emulsion is mixed with a solvent extraction phase to form microparticles in a liquid dispersion, wherein during residence in the plug flow reactor, a portion of the solvent is extracted into the extraction phase and the microparticles are hardened;
c) directly feeding the liquid dispersion to a thick wall hollow fiber tangential flow filter, wherein the thick wall hollow fiber tangential flow filter is in direct fluid communication with the plug flow reactor, wherein the tangential flow depth flow filter has a pore size of greater than 1 μm, and wherein a portion of the liquid dispersion containing solvent and microparticles below a specified-size threshold are removed as a permeate; and,
d) transferring the retentate to a holding tank.

20. The process of claim 19, further comprising (e), transferring the retentate back to the thick wall hollow fiber tangential flow filter via a loop circuit between the thick wall hollow fiber tangential flow filter and the holding tank.

21. The process of claim 19, wherein the liquid dispersion is mixed with additional solvent extraction phase at one or more locations within the plug flow reactor during its residence within the plug flow reactor.

22. The process of claim 19, wherein the thick wall hollow fiber tangential flow filter has a pore size of greater than 3 μm.

23. The process of claim 19, wherein the thick wall hollow fiber tangential flow filter has a pore size of greater than 5 μm.

24. The process of claim 19, wherein the thick wall hollow fiber tangential flow filter has a pore size of between 6 μm and 8 μm.

25. The process of claim 19, further comprising adding a surface treatment phase to liquid dispersion of microparticles in the plug flow reactor in step b).

26. The process of claim 19, further comprising adding a surface treatment phase to the retentate in the holding tank in step d).

27. A process of continuously producing a drug-loaded polymeric microparticle comprising:
a) continuously combining a dispersed phase and a continuous phase in a microfluidic droplet generator to produce droplets, wherein the dispersed phase comprises a drug, a polymer, and at least one solvent;
b) directly feeding the droplets into a plug flow reactor, wherein upon entering the plug flow reactor, the droplets are mixed with a solvent extraction phase, wherein during residence in the plug flow reactor, a portion of the solvent is extracted into the solvent extraction phase and the droplets are hardened to microparticles;
c) exposing the microparticles to surface-treatment solution in the plug flow reactor to produce surface-treated microparticles, and
d) directly feeding the surface-treated microparticles into a dilution vessel.

28. A process of continuously producing a drug-loaded polymeric microparticle comprising:
a) simultaneously combining a dispersed phase and a continuous phase in at least two microfluidic droplet generators to produce droplets, wherein the dispersed phase comprises a drug, a polymer, and at least one solvent;
b) directly feeding the droplets into a plug flow reactor, wherein upon entering the plug flow reactor, the droplets are mixed with a solvent extraction phase, wherein during residence in the plug flow reactor, a portion of the solvent is extracted into the solvent extraction phase and the droplets are hardened to microparticles;
c) exposing the microparticles to surface-treatment solution in the plug flow reactor to produce surface-treated microparticles, and
d) directly feeding the surface-treated microparticles into a dilution vessel.

29. The process of claim 27, wherein the microfluidic droplet generator further comprises a micro-mixing channel.

30. The process of claim 27, further comprising transferring the surface-treated microparticles from the dilution vessel to a continuous liquid centrifuge or a parallel bank of centrifuges via an outlet from the dilution vessel, wherein a portion of the liquid dispersion containing solvent and microparticles below a specified size threshold are removed with a waste solvent liquid and remaining microparticles above the specified size threshold are isolated as a concentrated slurry.

31. The process of claim 27, wherein the droplets in step (b) are mixed with additional solvent extraction phase at one or more locations within the plug flow reactor during their residence within the plug flow reactor.

32. The process of claim 27, wherein microparticles in step (c) are exposed to additional surface-treatment solution at one or more locations within the plug flow reactor during their residence in the plug flow reactor.

33. The process of claim 32, wherein microparticles in step (c) are exposed to surface-treatment solution for approximately 30 minutes or less.

34. The process of claim 27, wherein the plug flow reactor has a diameter of about 0.5 inches or less.

35. The process of claim 27, wherein one or more portions of the plug flow reactor are jacketed to maintain a temperature in the one or more portions of approximately 2-8° C.

36. The process of claim 8, wherein the surface treatment phase is NaOH in EtOH.

37. The process of claim 36, wherein the surface treatment phase is between 0.0075M NaOH/ethanol to 0.75M NaOH/ethanol

38. The process of claim 37, wherein the surface treatment phase is about 0.75M NaOH/EtOH.

39. The process of claim 1, wherein the drug is sunitinib or a pharmaceutically acceptable salt thereof.

40. The process of claim 39, wherein the pharmaceutically acceptable salt is sunitinib malate.
